(19) United States
(12) Patent Application Publication
(10) Pub. No.: US 2017/0362210 A1
(43) Pub. Date: Dec. 21, 2017

(54) ANTI-ESTROGENIC COMPOUNDS

(71) Applicant: PFIZER INC., NEW YORK, NY (US)

(72) Inventors: Peter J. KUSHNER, San Francisco, CA (US); David C. MYLES, Berkeley, CA (US); Cyrus L. HARMON, Bolinas, CA (US); Leslie Carol HODGES GALLAGHER, Alameda, CA (US)

(73) Assignee: PFIZER INC., NEW YORK, NY (US)

(21) Appl. No.: 15/676,400

(22) Filed: Aug. 14, 2017

Related U.S. Application Data
(63) Continuation of application No. 15/135,840, filed on Apr. 22, 2016.
(60) Provisional application No. 62/153,097, filed on Apr. 27, 2015.

(51) Int. Cl. C07D 405/12; C07D 405/10

(52) U.S. Cl. CPC C07D 405/12; C07D 405/10

(57) ABSTRACT

The present disclosure provides a compound of Formula I:

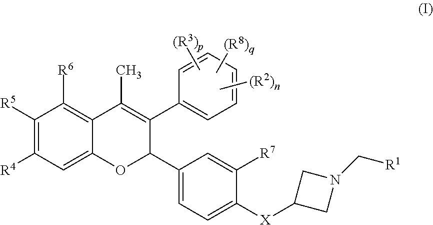

or a pharmaceutically acceptable salt wherein X, R¹-R⁸, Y¹-Y⁵, m, n, p, and q are defined herein. The novel 2H-chromene compounds are useful for the modulation of disorders mediated by estrogen, and other disorders, as described herein. The present invention also relates to pharmaceutical compositions containing the compounds and to methods of using the compounds and compositions.

### CROSS-REFERENCE TO RELATED APPLICATIONS

The present application is a continuation of U.S. Patent application Ser. No. 15/135,840 filed on Apr. 22, 2016, which claims priority of U.S. provisional application having U.S. Ser. No. 62/153,097, filed on Apr. 27, 2015.

### FIELD OF THE DISCLOSURE

The present invention relates to the field of pharmaceuticals, and in particular, to novel 2H-chromene compounds, salts, and prodrugs thereof. The present invention also relates to the medical uses of the compounds, including as estrogen receptor modulators, and for the treatment of medical conditions that would benefit from an anti-estrogenic drug, and pharmaceutical salts and compositions thereof.

### BACKGROUND

Estrogen receptor modulators are a class of compounds that act on the estrogen receptor. These compounds can be pure agonists (mimicking estrogen), pure antagonists, or mixed agonist-antagonists (sometimes referred to as Selective Estrogen Receptor Modulators (SERMs)). For example, estradiol is a pure agonist, fulvestrant is a complete antagonist, and tamoxifen and raloxifene are SERMs.

Most breast cancers express estrogen receptors (ER), and their growth is driven by the action of estrogen at its receptors, primarily at ER alpha. This type of cancer is treated with an estrogen antagonist, which competes with estrogen for binding to the receptor, but does not activate it, preventing estrogen driven growth. Partial anti-estrogens like raloxifene and tamoxifen retain some estrogen-like effects, including an estrogen-like stimulation of uterine growth, and also, in some cases, an estrogen-like action during breast cancer progression which actually stimulates tumor growth. In contrast, fulvestrant, a complete anti-estrogen, is free of estrogen-like action on the uterus and is effective in tamoxifen-resistant tumors. A recent study also suggests that fulvestrant is substantially superior to the aromatase inhibitor anastrozole in treating metastatic breast cancer (Robertson et al. J Clin Oncol (2009) 27(27):4530-5).

The degree of anti-estrogenicity is often assayed by exposing female, immature (preferably ovariectomized) rodents to test doses of the compound both in the absence (agonist mode) and presence (antagonist mode) of estrogen. Tamoxifen and other partial anti-estrogens stimulate uterine weight gain in the agonist mode and only partly block estrogen-driven uterine weight gain in the antagonist mode. Fulvestrant and other complete anti-estrogens do not stimulate uterine weight gain in the agonist mode and completely block estrogen-driven weight gain in the antagonist mode. The induction of estrogen-regulated alkaline phosphatase expression in human uterine cancer cell growth in culture can be used to distinguish partial and complete anti-estrogenicity and correlates well with the rodent weight gain assay.

Tamoxifen and fulvestrant both inhibit cultured human breast cancer cell proliferation provoked by estrogen. However, fulvestrant more fully inhibits the proliferation when provoked with growth factors, especially of the insulin/insulin-like growth factor family. Thus the inhibition of growth-factor driven breast cancer cell proliferation and the effect on uterine weight provide two assays which can distinguish between complete and partial anti-estrogens.

Compounds that act by degrading the estrogen receptor are sometimes referred to as “SERDs” (Selective Estrogen Receptor Degraders). While tamoxifen binding stabilizes the estrogen receptor, fulvestrant and chemically related antiestrogens, such as ICI-164384 and RU-58668, cause degradation of the estrogen receptor. The ability to induce degradation of the receptor is a factor that differentiates the behavior of tamoxifen and fulvestrant and may be desirable in a drug to treat breast cancer.

Fulvestrant incorporates a core of 17-beta estradiol. The estradiol core blocks oral absorption and the long flexible aliphatic side chain makes the drug very insoluble which worsens the problem. Fulvestrant must be injected because of its poor oral bioavailability. Two 5 ml intramuscular depot injections, one into each buttock, must be administered monthly by a health professional. Furthermore, it is unclear whether these two injections provide sufficient drug exposure for optimal action. The drug does not seem to work in pre-menopausal women.

Therefore, there is a need for new improved anti-estrogenic compounds for the treatment of medical disorders that are mediated or affected by an estrogen receptor and pharmaceutical compositions thereof.

### SUMMARY OF THE DISCLOSURE

Each of the embodiments described below can be combined with any other embodiment described herein not inconsistent with the embodiment with which it is combined. It is understood that all of the combinations and permutations of the definitions of X, R¹, R², R³, R⁴, R⁵, R⁶, R⁷, m, n, p, and q, are contemplated to be within the scope of the present disclosure. The phrase “or a pharmaceutically acceptable salt thereof” is implicit in the description of all compounds described herein; however, in one aspect of any of the embodiments herein, the compound is in the form of a free base.

Embodiments described herein relate to a compound of Formula I

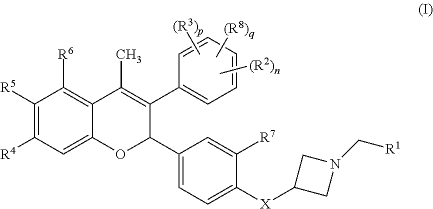

or a, pharmaceutically acceptable salt thereof,

wherein
X is CH₂, O, S, NH, or N—(C₁-C₄ alkyl);
R¹ is —(C(Y¹)(Y²))ₘ—C((Y³)(Y⁴)(Y⁵)), wherein Y¹, Y², Y³, Y⁴ and Y⁵ are independently hydrogen or fluorine;
m is 0 or 1;
R² is hydrogen, halogen, cyano, or hydroxy;
R³ is hydrogen, halogen, C₁-C₄ alkyl, —CH₂F, —CHF₂, or —CF₃;
R⁴ and R⁵ are each independently hydrogen, halogen, or hydroxy, provided that R⁴ and
R⁵ are not both hydroxy;
R⁶ and R⁷ are each independently hydrogen or halogen;
R⁸ is hydrogen, halogen, cyano, hydroxy, or C₁₋₄ alkyl;
n is 1 or 2;
p is 1 or 2; and
q is 1.

Embodiments described herein relate to a compound of Formula IA

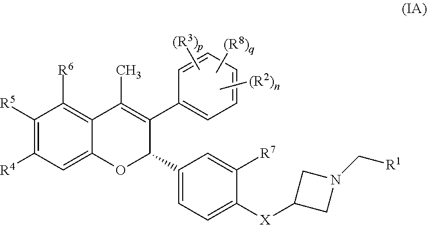

or a pharmaceutically acceptable salt thereof,

wherein R¹, R², R³, R⁴, R⁵, R⁶, R⁷, m, n, p and q are as defined hereinabove.

Embodiments described herein relate to a compound of Formula II

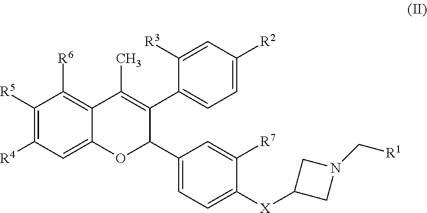

or a pharmaceutically acceptable salt thereof,

wherein
X is CH₂, O, S, NH, or N—(C₁-C₄ alkyl);
R¹ is —(C(Y¹)(Y²))ₘ—C((Y³)(Y⁴)(Y⁵)), wherein Y¹, Y², Y³, Y⁴ and Y⁵ are independently hydrogen or fluorine;
m is 0 or 1;
R² is hydrogen, halogen, cyano, or hydroxy;
R³ is hydrogen, halogen, C₁-C₄ alkyl, —CH₂F, —CHF₂, or —CF₃;
R⁴ and R are each independently hydrogen, halogen, or hydroxy, provided that R⁴ and
R⁵ are not both hydroxy; and
R⁶ and R⁷ are each independently hydrogen or halogen.

Embodiments described herein relate to a compound of Formula IIA

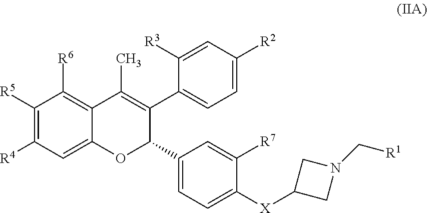

or a pharmaceutically acceptable salt thereof,

wherein R¹, R², R³, R⁴, R⁵, R⁶, R⁷, X and m are as defined hereinabove.

In an embodiment, X is CH₂ or O. In another embodiment, X is NH or N—(C₁-C₄ alkyl). In another embodiment, X is CH₂ or S. In another embodiment, X is O, and in a still further embodiment, X is CH₂, and in another embodiment, X is S.

In an embodiment, m is 0. In another embodiment, m is 1.

In an embodiment, R¹ is —C((Y¹)(Y²))ₘ—CH₃, where m and Y¹ and Y² are as defined herein. In a further embodiment, R¹ is —CH₂—CH₃. In another embodiment, R¹ is (CH₂)ₘ—C((Y³)(Y⁴)(Y⁵)), wherein m, Y³, Y⁴ and Y⁵ are as defined herein. In another embodiment, one or more of the hydrogen atoms attached to a carbon atom is replaced by fluorine. For example, in an embodiment, R is —(CH₂)ₘ—CH₂F, and in another embodiment, R¹ is —(CH₂)ₘ—CHF₂, while in another embodiment, R¹ is —(CH₂)ₘ—CF₃. In other embodiments, R¹ is CHF—CH₃, CF₂CH₃, CH₂CH₃, CH₂CH₂F, CH₂CHF₂, CH₂CF₃, CHFCH₂F, CHFCHF₂, CHFCF₃, CF₂CH₃, CF₂CH₂F, CF₂CF₂, CF₂CF₃, CH₂F, CHF₂, CH₃, or CF₃. In an embodiment, —C((Y³)(Y⁴)(Y⁵)), wherein Y³, Y⁴ and Y⁵ are independently hydrogen or fluorine. In an embodiment, R¹ is CH₂F, CHF₂, CH₃, or CF₃. In another embodiment, —C((Y³)(Y⁴)(Y⁵)) is CH₃ or CF₃.

In an embodiment, R² is hydrogen, halogen, such as F, Cl or Br, cyano or hydroxy, while in another embodiment, R² is hydrogen, halogen or hydroxy. In a further embodiment, R² is hydrogen or hydroxy. In an embodiment, R² is hydrogen. In another embodiment, R² is hydroxy.

In an embodiment, R³ is hydrogen, F, Br, Cl, methyl, ethyl, n-propyl, isopropyl, n-butyl, isobutyl, sec-butyl, tert-butyl, —CH₂F, —CHF₂, or —CF₃. In another embodiment, R³ is hydrogen or C₁-C₄ alkyl. In another embodiment, R³ is methyl, isopropyl or hydrogen. In another embodiment, R³ is hydrogen or isopropyl. In a further embodiment, R³ is methyl. In another embodiment, R³ is hydrogen.

In an embodiment, R⁴ is hydroxy and R⁵ is hydrogen or halogen, for example, bromine, fluorine or iodine. In another embodiment, R⁵ is hydroxy, and R is hydrogen or halogen, for example, bromine, fluorine or iodine. In another embodiment, R⁴ is hydroxy and R⁵ is hydrogen, while in another embodiment, R⁵ is hydroxy and R⁴ is hydrogen. In another embodiment, R⁴ is hydroxy. In another embodiment, R⁵ is hydrogen or halogen. In another embodiment, R⁵ is hydrogen.

In an embodiment, R⁶ is hydrogen or bromine, fluorine or iodine, and in another embodiment, is hydrogen or fluorine or chlorine, and in another embodiment, is hydrogen or fluorine, and in still further embodiment, is hydrogen and in still another embodiment, is fluorine.

In an embodiment, R⁷ is hydrogen or bromine, fluorine or iodine, and in another embodiment, is hydrogen or fluorine or chlorine, and in another embodiment, is hydrogen or fluorine, and in still further embodiment, is hydrogen and in still another embodiment, is fluorine.

In an embodiment, R⁶ is hydrogen, R⁷ is hydrogen or fluorine, and R⁸ is hydrogen. In another embodiment, R⁶ is hydrogen or fluorine, R⁷ is fluorine, and R⁸ is hydrogen. In an embodiment, R⁶ is hydrogen and R⁷ is hydrogen or fluorine. In another embodiment, R⁶ is hydrogen or fluorine and R⁷ is fluorine. In an embodiment, R⁶ is hydrogen, R⁷ is hydrogen, and R⁸ is hydrogen.

In an embodiment, X is CH₂, R¹ is CH₂—CH₃ or CH₂—CF₃, R² is hydroxy, R³ is hydrogen, one of R⁴ and R⁵ is hydroxy and the other is hydrogen, R⁶ is hydrogen or fluorine, R⁷ is hydrogen or fluorine and R⁸ is hydrogen. In another embodiment, X is CH₂, R¹ is CH₂—CH₃ or CH₂—CF₃, R² is hydroxy, R³ is hydrogen, one of R⁴ and R⁵ is hydroxy and the other is hydrogen, R⁶ is hydrogen or fluorine, and R⁷ is hydrogen or fluorine.

In an embodiment, X is O, R is CH₂—CH₃ or CH₂—CF₃, R² is hydroxy, R³ is hydrogen, one of R⁴ and R⁵ is hydroxy and the other is hydrogen, R⁶ is hydrogen, R⁷ is hydrogen or fluorine, and R⁸ is hydrogen. In another embodiment, X is O, R¹ is CH₂—CH₃ or CH₂—CF₃, R² is hydroxy, R³ is hydrogen, one of R⁴ and R⁵ is hydroxy and the other is hydrogen, R⁶ is hydrogen and R⁷ is hydrogen or fluorine.

In an embodiment, X is CH₂, R¹ is CH₃ or CF₃, R² is hydroxy, R³ is hydrogen, one of R⁴ and R⁵ is hydroxy and the other is hydrogen, R⁶ is hydrogen or fluorine, R⁷ is hydrogen or fluorine and R⁸ is hydrogen. In another embodiment, X is CH₂, R¹ is CH₃ or CF₃, R² is hydroxy, R³ is hydrogen, one of R⁴ and R⁵ is hydroxy and the other is hydrogen, R⁶ is hydrogen or fluorine, and R⁷ is hydrogen or fluorine,

In an embodiment, X is O, R¹ is CH₃ or CF₃, R² is hydroxy, R³ is hydrogen, one of R⁴ and R⁵ is hydroxy and the other is hydrogen, R⁶ is hydrogen, R⁷ is hydrogen or fluorine, and R⁸ is hydrogen. In another embodiment, X is O, R¹ is CH₃ or CF₃, R² is hydroxy, R³ is hydrogen, one of R⁴ and R⁵ is hydroxy and the other is hydrogen, R⁶ is hydrogen and R⁷ is hydrogen or fluorine.

Embodiments described herein relate to a compound of Formula I wherein the aryl moiety substituted by R², R³ and R is selected from:

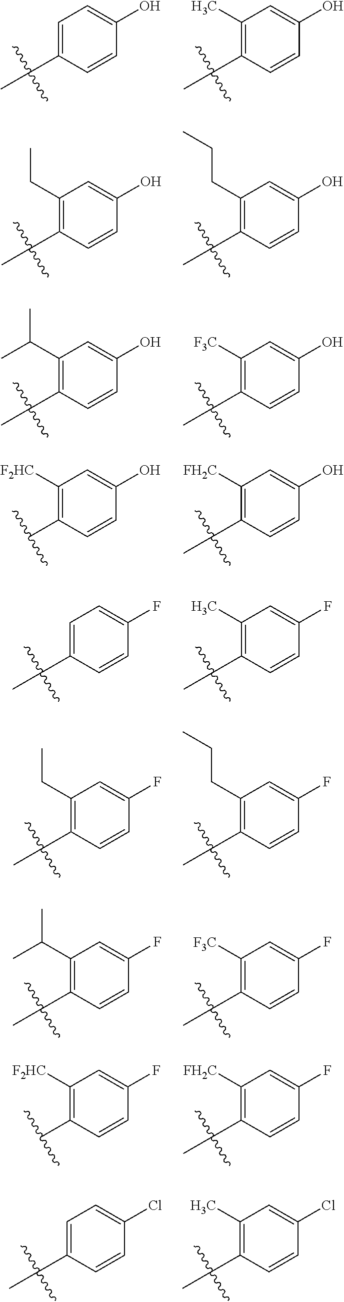

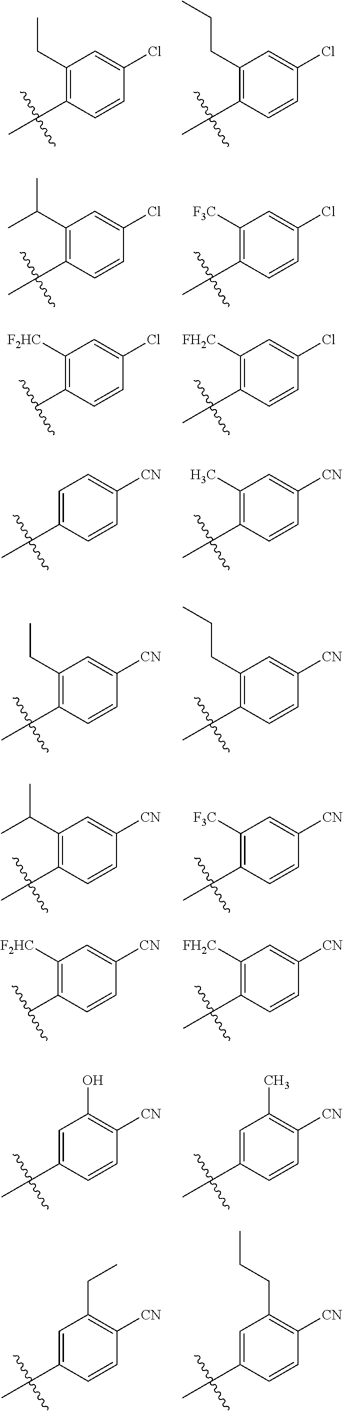

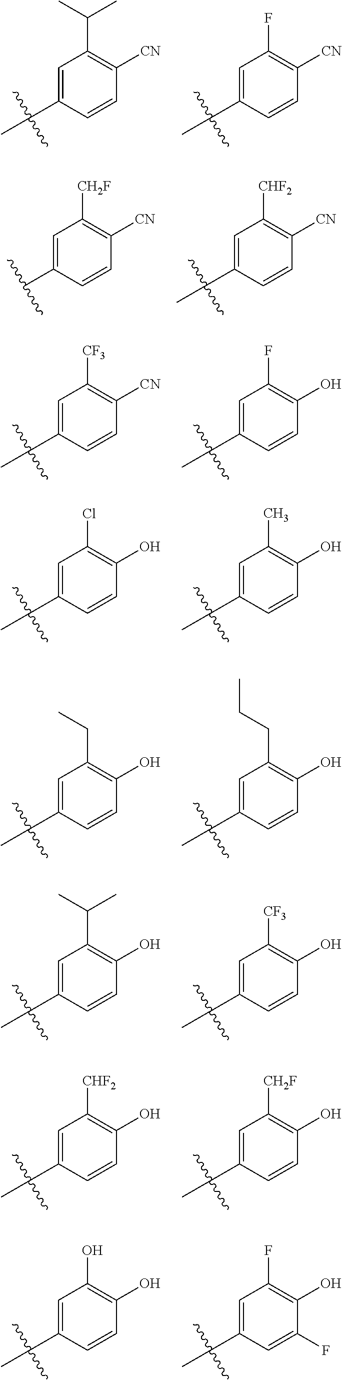

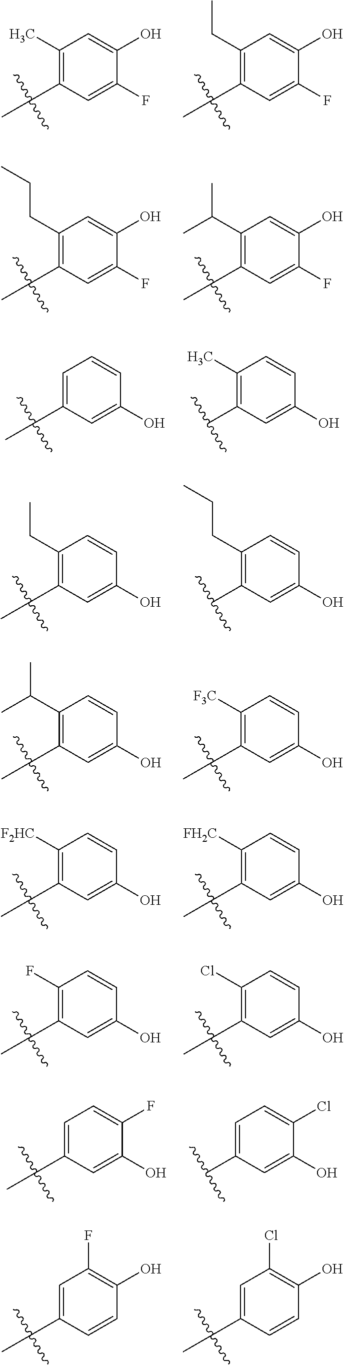

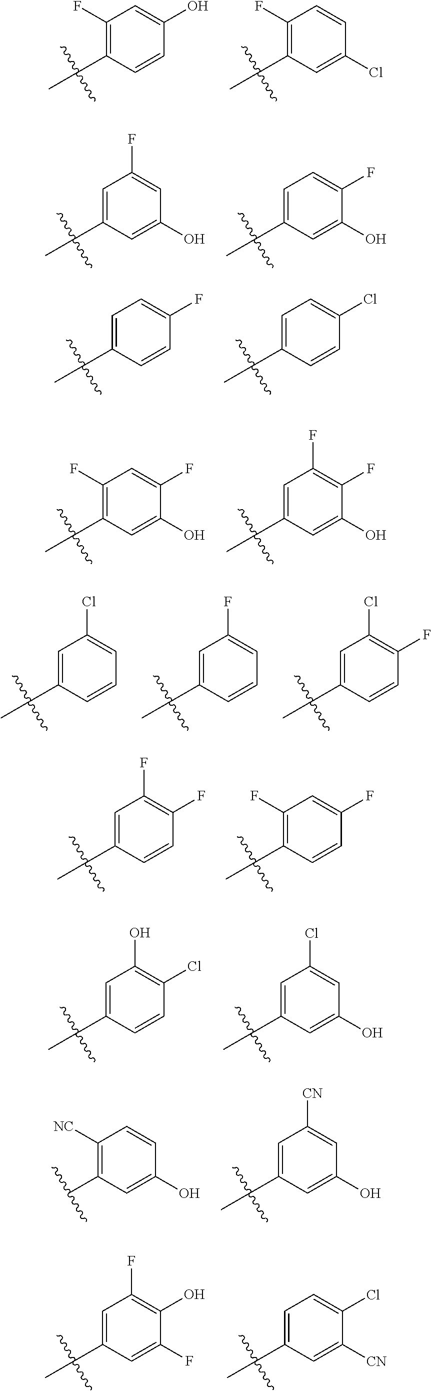

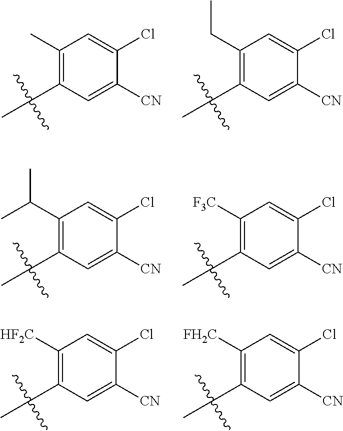

In an embodiment, a compound is selected from the group consisting of
3-(4-hydroxyphenyl)-4-methyl-2-(4-((1-propylazetidin-3-yl)methyl)phenyl)-2H-chromen-7-ol;
3-(4-hydroxyphenyl)-4-methyl-2-(4 ((1-propylazetidin-3-yl)oxy)phenyl)-2H-chromen-7-ol;
2-(3-fluoro-4-((1-propylazetidin-3-yl)oxy)phenyl)-3-(4-hydroxyphenyl)-4-methyl-2H-chromen-7-ol;
3-(4-hydroxyphenyl)-4-methyl-2-(4-((1-(3,3,3-trifluoropropyl)azetidin-3-yl)oxy)phenyl)-2H-chromen-7-ol;
2-(3-fluoro-4-((1-propylazetidin-3-yl)methyl)phenyl)-3-(4-hydroxyphenyl)-4-methyl-2H-chromen-7-ol;
4-methyl-3-phenyl-2-(4-((1-propylazetidin-3-yl)oxy)phenyl)-2H-chromen-7-ol;
3-(4-fluorophenyl)-4-methyl-2-(4-((1-propylazetidin-3-yl)oxy)phenyl)-2H-chromen-7-ol;
3-(2-chloro-4-fluorophenyl)-4-methyl-2-(4-((1-propylazetidin-3-yl)oxy)phenyl)-2H-chromen-7-ol;
3-(2-isopropylphenyl)-4-methyl-2-(4-((1-propylazetidin-3-yl)oxy)phenyl)-2H-chromen-7-ol; and
3-(2-chlorophenyl)-4-methyl-2-(4-((1-propylazetidin-3-yl)oxy)phenyl)-2H-chromen-7-ol,
   or a pharmaceutically acceptable salt thereof.

In an embodiment, a compound is selected from the group consisting of
3-(4-hydroxyphenyl)-4-methyl-2-(4-((1-propylazetidin-3-yl)methyl)phenyl)-2H-chromen-7-ol;
3-(4-hydroxyphenyl)-4-methyl-2-(4-((1-propylazetidin-3-yl)oxy)phenyl)-2H-chromen-7-ol;
2-(3-fluoro-4-((1-propylazetidin-3-yl)oxy)phenyl)-3-(4-hydroxyphenyl)-4-methyl-2H-chromen-7-ol;
3-(4-hydroxyphenyl)-4-methyl-2-(4-((1-(3,3,3-trifluoropropyl)azetidin-3-yl)oxy)phenyl)-2H-chromen-7-ol; and
2-(3-fluoro-4-((1-propylazetidin-3-yl)methyl)phenyl)-3-(4-hydroxyphenyl)-4-methyl-2H-chromen-7-ol,

or a pharmaceutically acceptable salt thereof.

In an embodiment, a compound is selected from the group consisting of

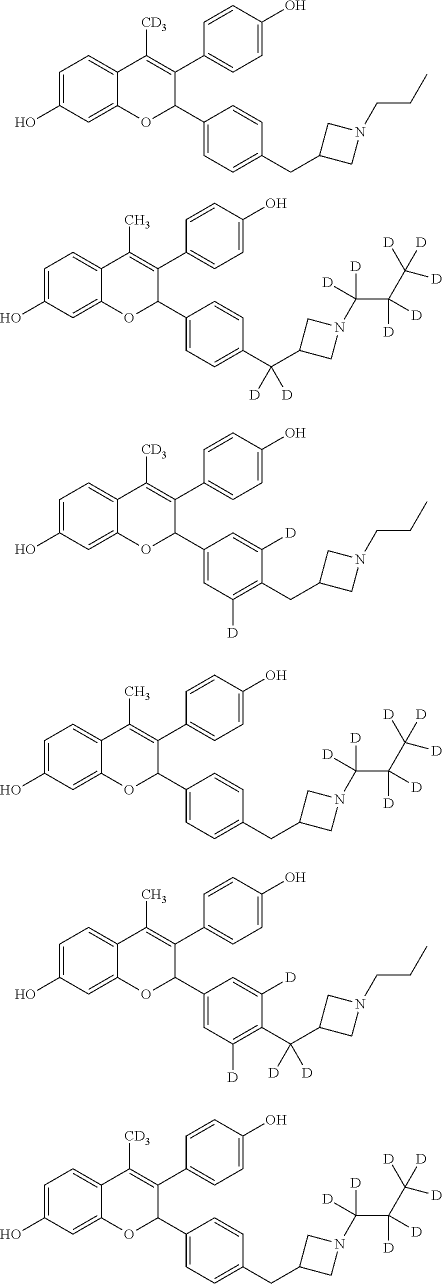

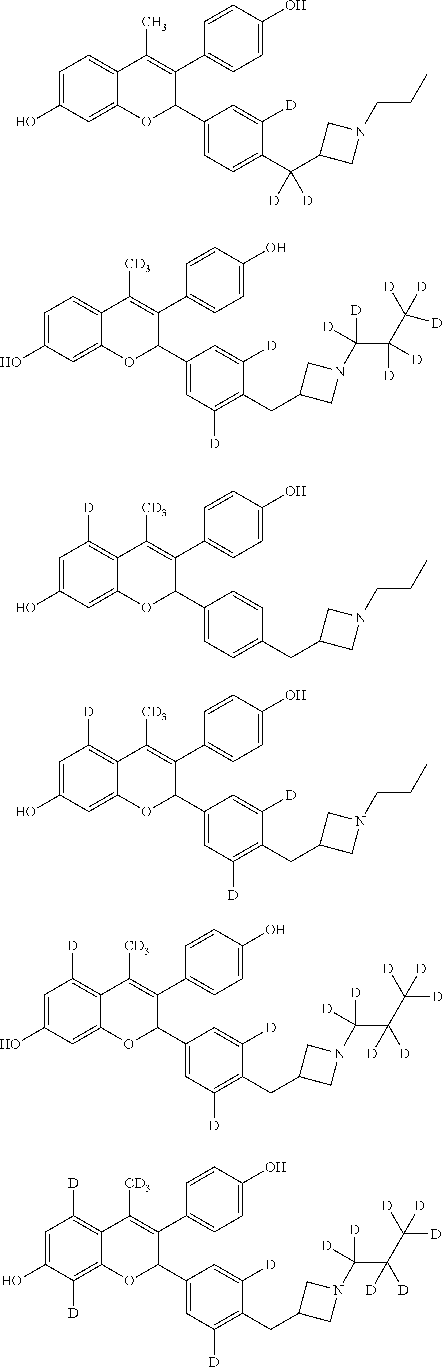

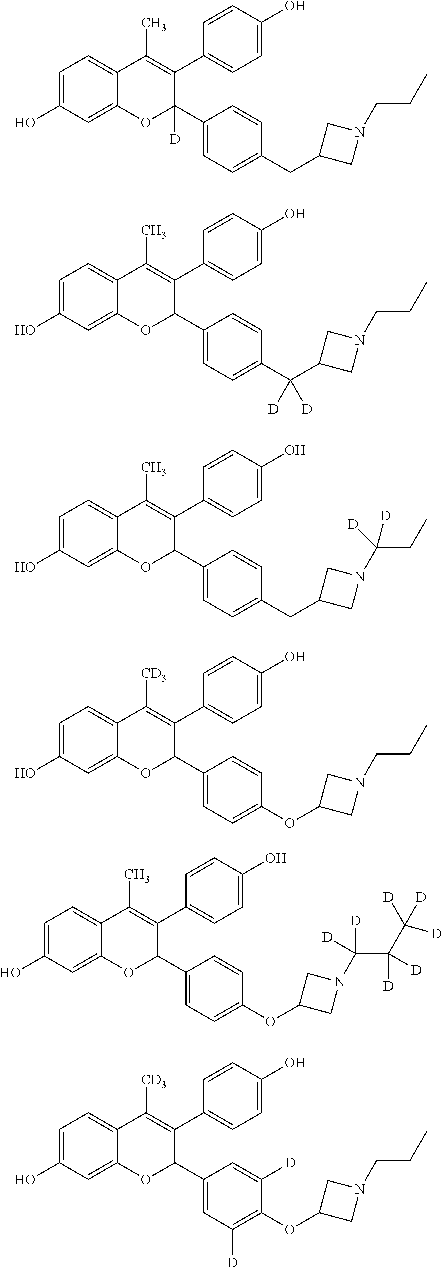

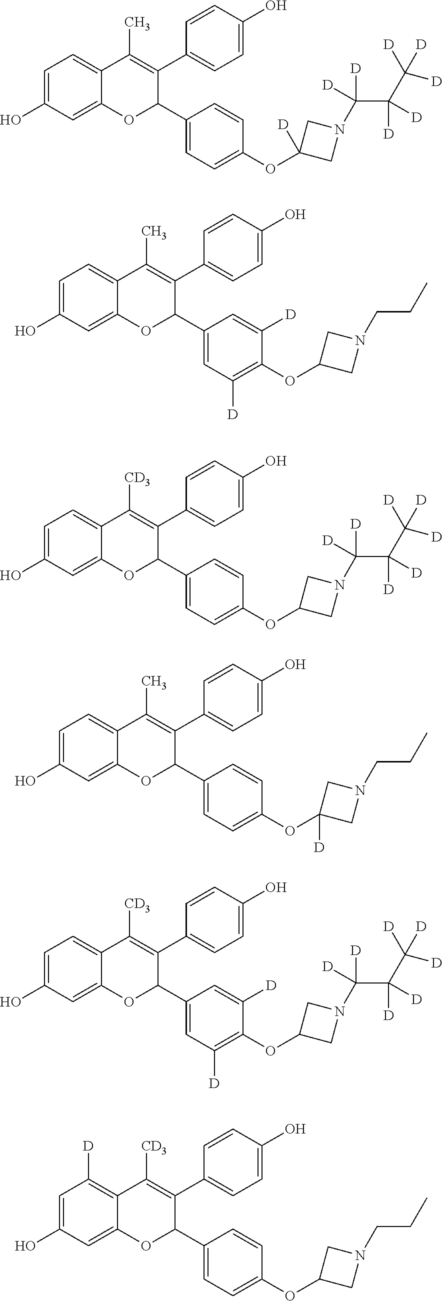

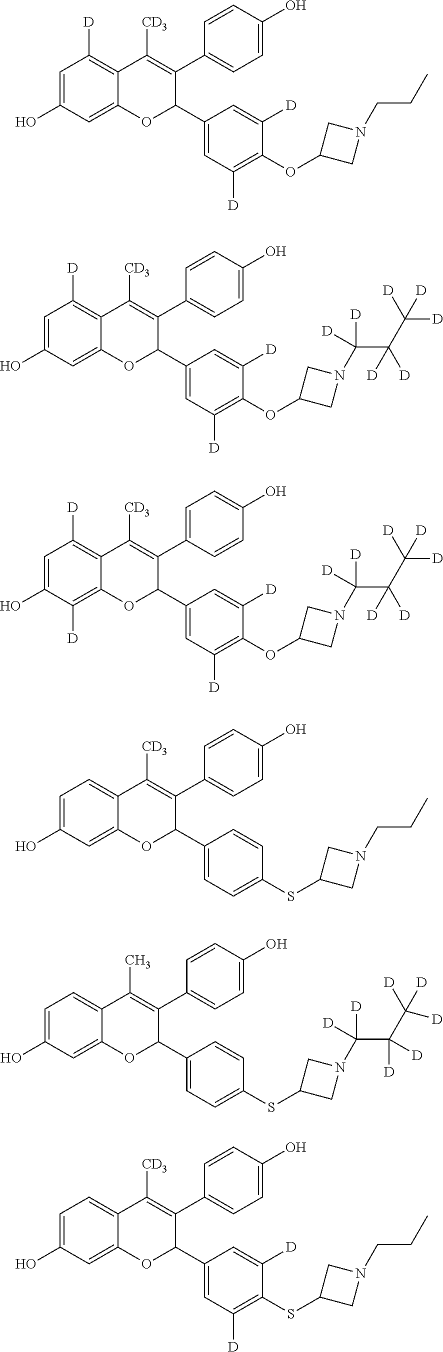

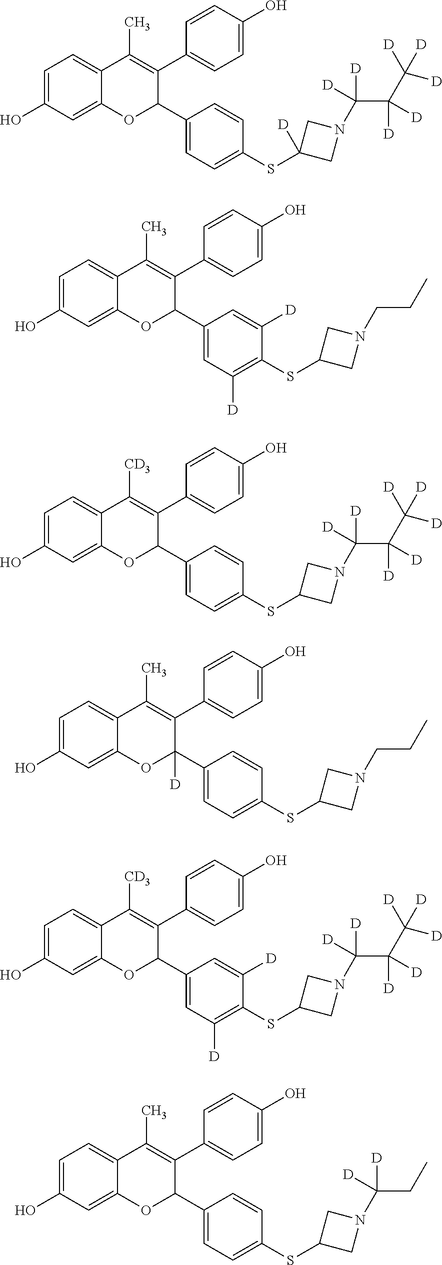

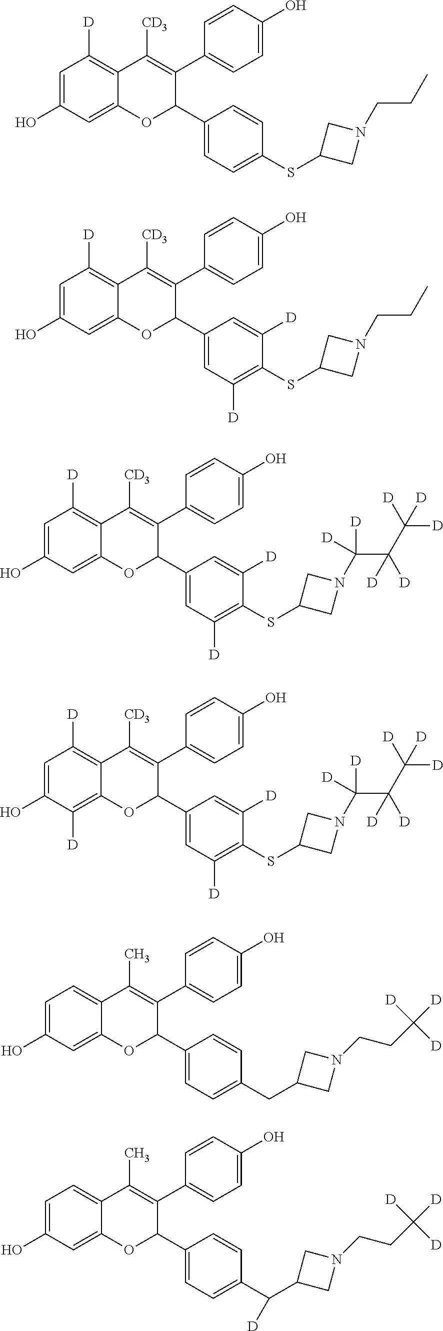

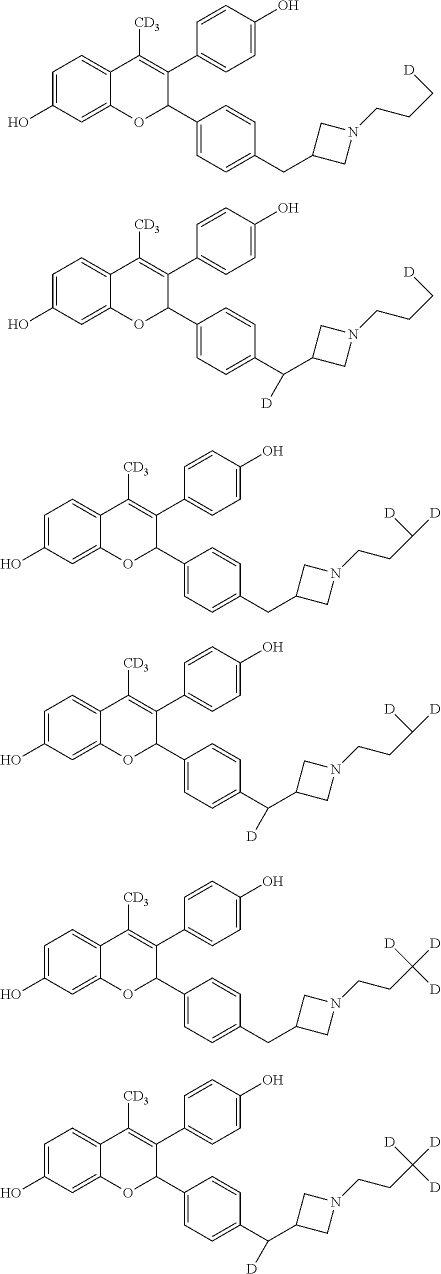

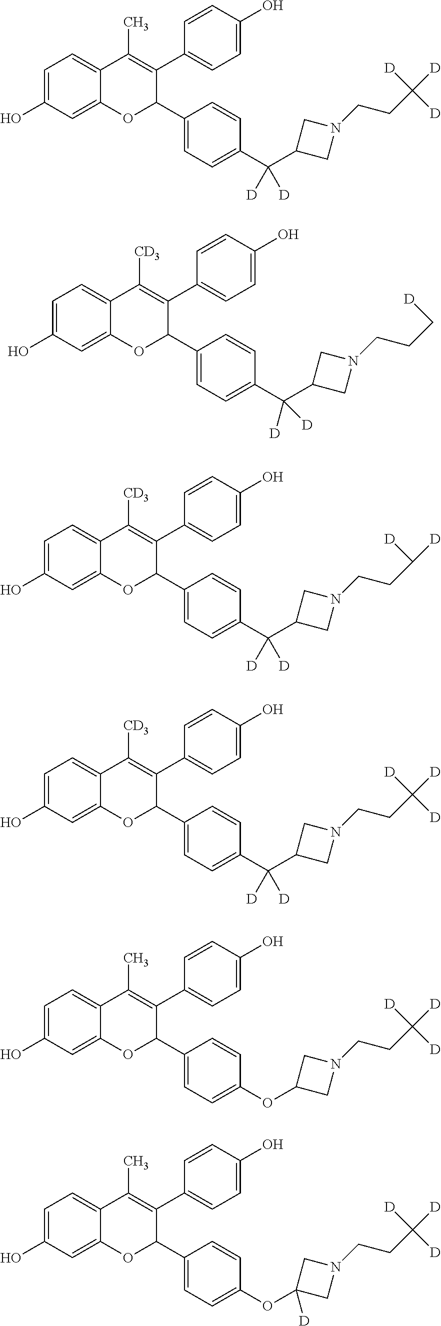

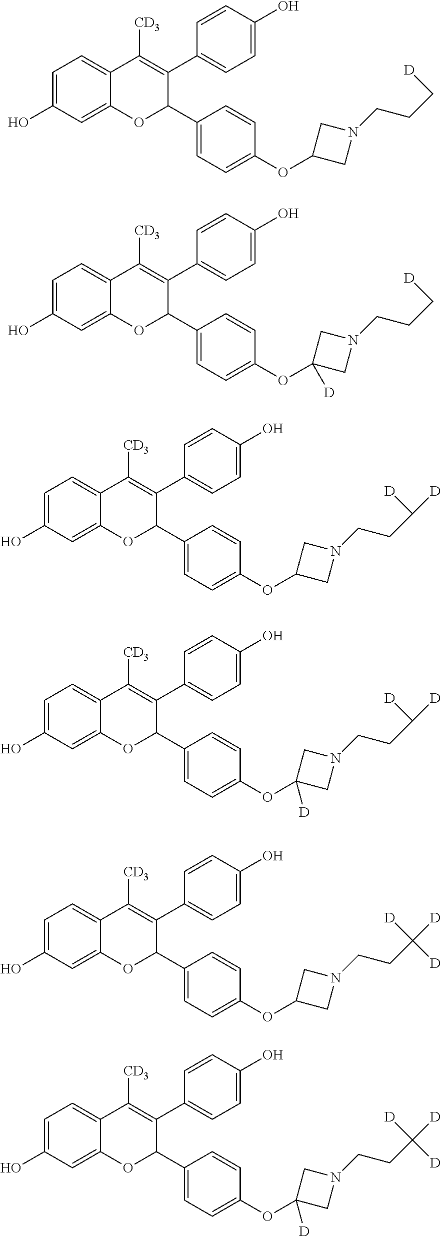

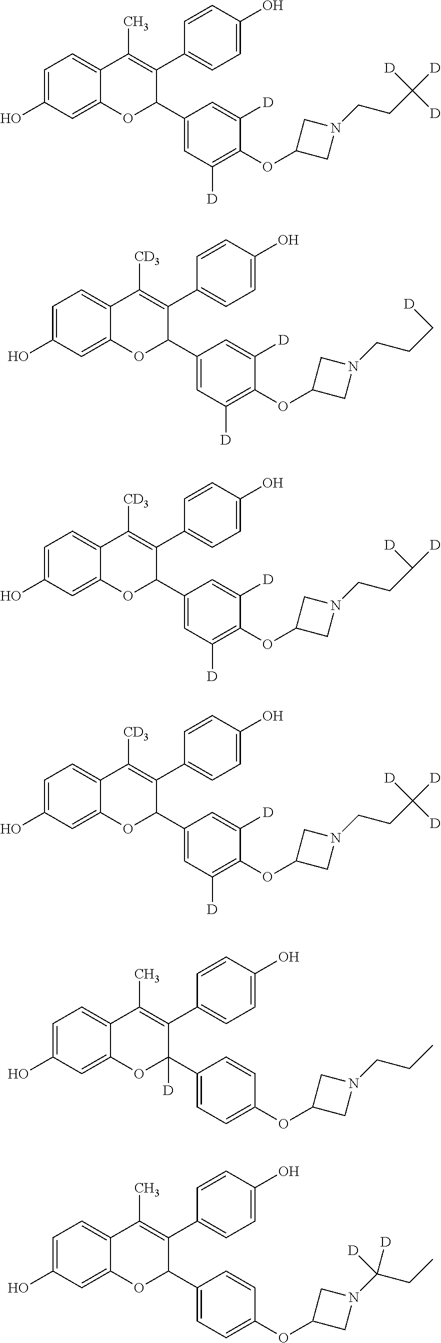

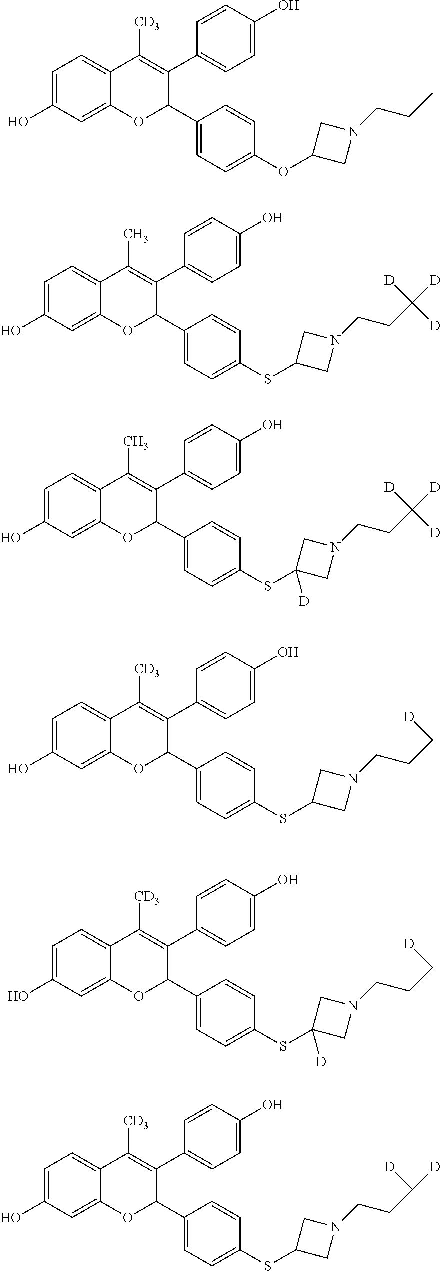 [SMILES: [2H]C([2H])([2H])C1=C(c2ccc(O)cc2)C(c2ccc(OC3CN(CCC)C3)cc2)Oc2cc(O)ccc21 ; [2H]C([2H])([2H])CCN1CC(Sc2ccc(C3Oc4cc(O)ccc4C(C)=C3c3ccc(O)cc3)cc2)C1 ; [2H]C([2H])([2H])CCN1CC([2H])(Sc2ccc(C3Oc4cc(O)ccc4C(C)=C3c3ccc(O)cc3)cc2)C1 ; [2H]CCCN1CC(Sc2ccc(C3Oc4cc(O)ccc4C(C([2H])([2H])[2H])=C3c3ccc(O)cc3)cc2)C1 ; [2H]CCCN1CC([2H])(Sc2ccc(C3Oc4cc(O)ccc4C(C([2H])([2H])[2H])=C3c3ccc(O)cc3)cc2)C1 ; [2H]C([2H])CCN1CC(Sc2ccc(C3Oc4cc(O)ccc4C(C([2H])([2H])[2H])=C3c3ccc(O)cc3)cc2)C1]

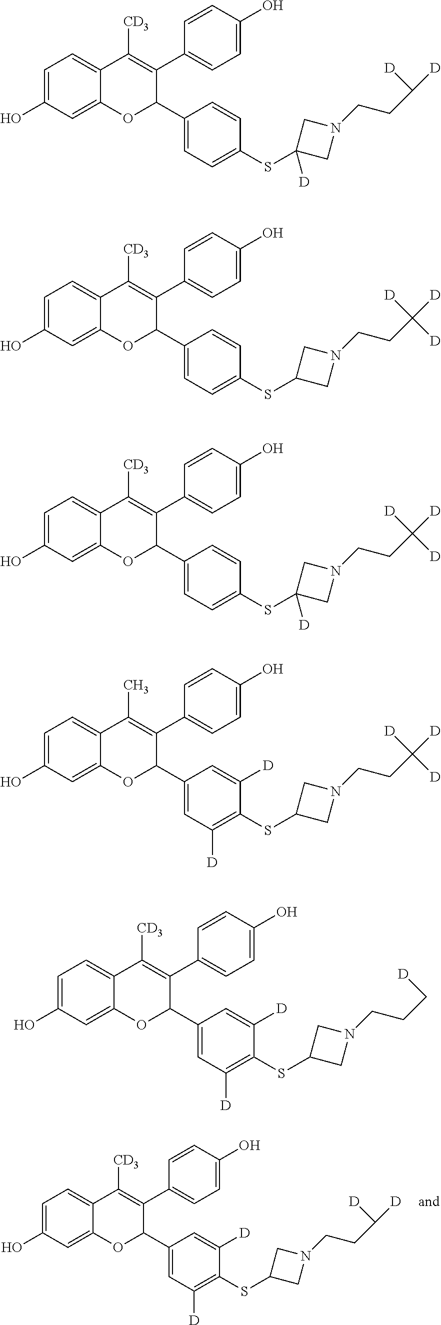

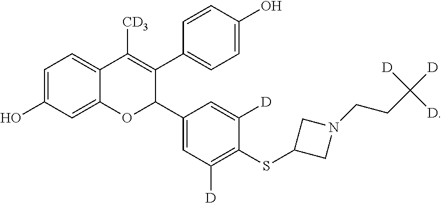

In an embodiment, a compound of the present invention is in the S configuration at the asymmetric carbon, as described below.

Embodiments relate to a pharmaceutical composition comprising a compound of any of the embodiments of Formula I, Formula IA, Formula II, or Formula IIA, or a pharmaceutically acceptable salt thereof, and a pharmaceutically acceptable carrier or diluent.

Embodiments relate to a pharmaceutical composition comprising a compound of any of the embodiments of Formula I, Formula IA, Formula II, or Formula IIA, or a pharmaceutically acceptable salt thereof, and a pharmaceutically acceptable carrier.

Embodiments relate to a method for treating a disorder mediated by the estrogen receptor in a patient, which comprises administering to the patient an amount of a compound of any of the embodiments of Formula I, Formula IA, Formula II, or Formula IIA, or a pharmaceutically acceptable salt thereof.

Embodiments related to the method of treating a disorder, wherein the disorder is breast cancer.

Embodiments related to the method of treating a disorder, wherein the disorder is selected from the group consisting of ovarian, endometrial, vaginal cancer, endometriosis or lung cancer.

Embodiments related to the method of treating a disorder mediated by the estrogen receptor, further comprising administering the compound in combination or alternation with another anti-cancer agent for the treatment of cancer.

Embodiments related to the method of treating a disorder mediated by the estrogen receptor, further comprising administering the compound in combination or alternation with estrogen or a partial estrogen receptor antagonist for the treatment of a postmenopausal disorder.

In an embodiment, the compound of the present specification of Formula I, IA, II or IIA can be provided if desired as a pharmaceutically acceptable salt, solvate, hydrate, prodrug, stereoisomer, tautomer, or N-oxide, and optionally in a pharmaceutically acceptable composition to treat a disorder that is modulated or affected by an estrogen receptor, including those treatable with an anti-estrogenic compound. In one embodiment, the compound of Formula I, Formula IA, Formula II or Formula IIA is provided as a prodrug, for example, an ester, ether, amide, carbonate or phosphate.

In another embodiment, the compound of Formula I, Formula IA, Formula II or Formula IIA has at least one isotopic substitution, and in particular, for example, at least one substitution of deuterium for hydrogen. In certain embodiments, deuterium in place of a hydrogen at one or more of the positions of the Formulas are provided.

Examples of disorders that can be treated with the compounds described herein include, but are not limited to, locally advanced or metastatic breast cancer that is positive for expression of estrogen receptors, progesterone receptors or both, and to early (surgically treatable) estrogen or progesterone receptor positive breast cancer, for treatment of which the compounds may be administered prior to surgery or following surgery to decrease the risk of recurrence. The described compounds or their pharmaceutically acceptable salts or compositions are therefore useful as adjunctive therapy after or instead of chemotherapy, radiation or surgery. They are also useful for the prevention of breast cancer in women at high risk or for treatment of other cancers and other overgrowth diseases of estrogen-receptive tissue, such as the female reproductive tract including ovarian, endometrial, vaginal cancer and endometriosis.

### DETAILED DESCRIPTION OF THE DISCLOSURE

The foregoing general description and the following detailed description are exemplary and explanatory only and are not restrictive of the invention. Other features and benefits of any one or more of the embodiments will be apparent from the following detailed description, and from the claims.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

Also, use of “a” or “an” are employed to describe elements and components described herein. This is done merely for convenience and to give a general sense of the scope of the invention. This description should be read to include one or at least one and the singular also includes the plural unless it is obvious that it is meant otherwise.

Unless otherwise defined, all technical and scientific terms used herein have the same meaning as commonly understood by one of ordinary skill in the art to which this invention belongs. In case of conflict, the present specification, including definitions, will control.

Although methods and materials similar or equivalent to those described herein can be used in the practice or testing of embodiments of the present invention, suitable methods and materials are described below. In addition, the materials, methods, and examples are illustrative only and not intended to be limiting.

When an amount, concentration, or other value or parameter is given as either a range, preferred range or a list of upper preferable values and/or lower preferable values, this is to be understood as specifically disclosing all ranges formed from any pair of any upper range limit or preferred value and any lower range limit or preferred value, regardless of whether ranges are separately disclosed. Where a range of numerical values is recited herein, unless otherwise stated, the range is intended to include the endpoints thereof, and all integers and fractions within the range.

The term “C₁-C₄ alkyl” refers to an alkyl group which contains 1-4 carbon atoms. The alkyl group may be a straight-chained, branched or cyclic. Examples include methyl, ethyl, n-propyl, isopropyl, n-butyl, isobutyl, sec-butyl, tert-butyl, cyclopropyl, and cyclobutyl.

The term “halogen” refers to fluorine, chlorine, bromine or iodine, and in particular, fluorine.

“Pharmaceutically acceptable salt” refers to a salt of a compound of the invention that is pharmaceutically acceptable and that possesses the desired pharmacological activity of the parent compound. In particular, such salts are non-toxic, and may be inorganic or organic acid addition salts and base addition salts. Specifically, such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl) benzoic acid, cinnamic acid, mandelic acid, methanesulfonic acid, ethanesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, N-methylglucamine and the like. Salts further include, by way of example only, sodium, potassium, calcium, magnesium, ammonium, tetraalkylammonium, and the like; and when the compound contains a basic functionality, salts of non-toxic organic or inorganic acids, such as hydrochloride, hydrobromide, tartrate, mesylate, acetate, maleate, oxalate and the like. The term “pharmaceutically acceptable cation” refers to an acceptable cationic counter-ion of an acidic functional group. Such cations are exemplified by sodium, potassium, calcium, magnesium, ammonium, tetraalkylammonium cations, and the like (see, e.g., Berge, et al., J. Pharm. Sci. 66(1): 1-79 (January '77).

“Pharmaceutically acceptable carrier” refers to a diluent, adjuvant, excipient or carrier with which a compound of the invention is administered.

“Solvate” refers to forms of the compound that are associated with a solvent or water (also referred to as “hydrate”), usually by a solvolysis reaction. This physical association includes hydrogen bonding. Conventional solvents include water, ethanol, acetic acid and the like. The compounds of the invention may be prepared e.g. in crystalline or liquid form and may be solvated or hydrated. Suitable solvates include pharmaceutically acceptable solvates, such as hydrates, and further include both stoichiometric solvates and non-stoichiometric solvates. In certain instances the solvate will be capable of isolation, for example when one or more solvent molecules are incorporated in the crystal lattice of the crystalline solid. “Solvate” encompasses both solution-phase and isolable solvates. Representative solvates include hydrates, ethanolates and methanolates.

A “subject” or “patient” to which administration is contemplated includes, but is not limited to, humans (i.e., a male or female of any age group, e.g., a pediatric subject (e.g., infant, child, adolescent) or adult subject (e.g., young adult, middle-aged adult or senior adult)). In an alternative embodiment, the subject can be a non-human animal, e.g., a mammal such as primates (e.g., cynomolgus monkeys, rhesus monkeys), cattle, pig, horse, sheep, goat, rodent, cat, and/or dog. Unless otherwise indicated, the term subject in a claim refers to a human.

As used herein the term “enantiomerically pure” or “pure enantiomer” denotes that the compound in the specific enantiomer, whether it be the R isomer or the S isomer, comprises more than 95% by weight. In alternative embodiments, the term may refer to more than 96% by weight, more than 97% by weight, more than 98% by weight, more than 98.5% by weight, more than 99% by weight, more than 99.2% by weight, more than 99.5% by weight, more than 99.6% by weight, more than 99.7% by weight, more than 99.8% by weight or more than 99.9% by weight, of the enantiomer. The weights are based upon total weight of all enantiomers or stereoisomers of the compound.

As used herein the term “diastereomerically pure” or “pure diastereomer” denotes that the compound in the specific diastereomer, comprises approximately 95% or more by weight.

In alternative embodiments, the term may refer to more than 96% by weight, more than 97% by weight, more than 98% by weight, more than 98.5% by weight, more than 99% by weight, more than 99.2% by weight, more than 99.5% by weight, more than 99.6% by weight, more than 99.7% by weight, more than 99.8% by weight or more than 99.9% by weight, of the diastereomer. The weights are based upon total weight of all stereoisomers of the compound.

As used herein and unless otherwise indicated, the term “enantiomerically pure R-compound” refers to at least about 95% by weight R-compound and at most about 5% by weight S-compound. In alternative embodiments, the term can refer to at least about 99% by weight R-compound and at most about 1% by weight S-compound or at least about 99.9% by weight R-compound or at most about 0.1% by weight S-compound. In certain embodiments, the weights are based upon total weight of compound.

As used herein and unless otherwise indicated, the term “enantiomerically pure S-compound” or “S-compound” refers to at least about 95% by weight S-compound and at most about 5% by weight R-compound. In alternative embodiments, the term can refer to at least about 99% by weight S-compound and at most about 1% by weight R-compound or at least about 99.9% by weight S-compound and at most about 0.1% by weight R-compound. In certain embodiments, the weights are based upon total weight of compound.

The term “therapeutically effective amount” means an amount of the composition's active component sufficient to treat a particular condition.

### Isotopic Substitution

The present invention includes the compounds of Formulas I, IA, II or HA and the use of compounds with desired isotopic substitutions of atoms, at an amount above the natural abundance of the isotope, i.e., enriched. Isotopes are atoms having the same atomic number but different mass numbers, i.e., the same number of protons but a different number of neutrons. By way of general example and without limitation, isotopes of hydrogen, for example, deuterium (²H) and tritium (³H) may be used anywhere in described structures. Alternatively or in addition, isotopes of carbon, e.g., ¹³C and ¹⁴C, may be used. A preferred isotopic substitution is deuterium for hydrogen at one or more locations on the molecule to improve the performance of the drug. The deuterium can be bound in a location of bond breakage during metabolism (an α-deuterium kinetic isotope effect) or next to or near the site of bond breakage (a β-deuterium kinetic isotope effect).

Substitution with isotopes such as deuterium can afford certain therapeutic advantages resulting from greater metabolic stability, such as, for example, increased in vivo half-life or reduced dosage requirements. Substitution of deuterium for hydrogen at a site of metabolic break down can reduce the rate of or eliminate the metabolism at that bond. At any position of the compound that a hydrogen atom may be present, the hydrogen atom can be any isotope of hydrogen, including protium (¹H), deuterium (²H) and tritium (³H). Thus, reference herein to a compound encompasses all potential isotopic forms unless the context clearly dictates otherwise.

The term “isotopically-labeled” analog refers to an analog that is a “deuterated analog”, a “¹³C-labeled analog,” or a “deuterated/¹³C-labeled analog.” The term “deuterated analog” means a compound described herein, whereby an H-isotope, i.e., hydrogen/protium (¹H), is substituted by an H-isotope, i.e., deuterium (²H). Deuterium substitution can be partial or complete. Partial deuterium substitution means that at least one hydrogen is substituted by at least one deuterium. In certain embodiments, the isotope is 90, 95 or 99% or more enriched in an isotope at any location of interest. In some embodiments it is deuterium that is 90, 95 or 99% enriched at a desired location.

It is understood that aspects and variations of the invention described herein include “consisting of” and/or “consisting essentially of” aspects and variations.

In an embodiment, the compounds of the Formulae described herein are substantially pure. By use of the term “substantially pure”, it is meant for example that the compounds of Formula I, IA, II and Formula IIA are at least about 80% by weight pure. In another embodiment, the compound of Formula I, IA, II and Formula IIA is at least about 85% by weight pure, while in another embodiment, it is at least about 90% by weight pure. In still another embodiment, the term “substantially pure” means that the compound of Formula II and Formula IIA is at least about 95% pure by weight. In another embodiment, it is at least about 97% pure by weight, and in another embodiment, it is at least about 98% and in still another embodiment, it is at least about 99% pure by weight. Unless otherwise indicated, the term substantially pure means at least about 90% by weight.

### Forms of Compounds

The compounds of Formula I, Formula IA, Formula II, Formula IIA and Formula III include stereoisomers thereof, including, without limitation, enantiomers, diastereomers and racemic mixtures thereof, unless the chemical structure depicts a certain stereo configuration.

In that case, the corresponding enantiomer, diastereomer or racemic mixture may be used in an alternative embodiment.

In particular, it is noted that the carbon atom at the 2-position of the chromene backbone of the compounds of Formula I or Formula IA which is bonded to the phenyl group, is an asymmetric carbon; thus, it may exist in either the R or S configuration. The present disclosure includes the R-isomer at the 2-position of the chromene, the S-isomer at the 2-position of the chromene, or a mixture thereof in any ratio, including a racemic mixture.

Unless indicated to the contrary herein, reference to the “S isomer” of a compound, refers to the compounds described herein in which the 2-position of the chromene is in the S configuration. Similarly, unless indicated to the contrary herein, reference to the “R isomer” of a compound, refers to the compounds described herein in which the 2-position of the chromene is in the R configuration.

In certain embodiments, deuterium in place of a hydrogen at one or more of the positions of Formula III are provided. Formula III illustrates the 19 positions that can be deuterated. Carbon-hydrogen bonds that can be broken during metabolism: 1, 5, 6 (when R³ is alkyl), 7, 8, 9, 10, 11, 12, 13, 14, 15, 16, 17, 18 and 19. Secondary deuterium isotope effects can be effected at positions 16, 17, 18 and 19.

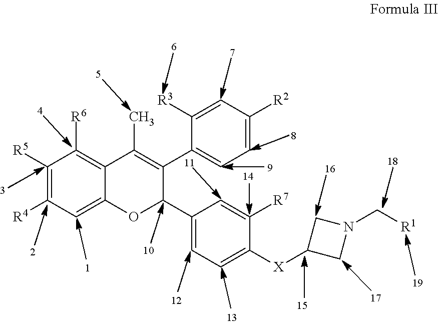

Examples of compounds of Formula III include:

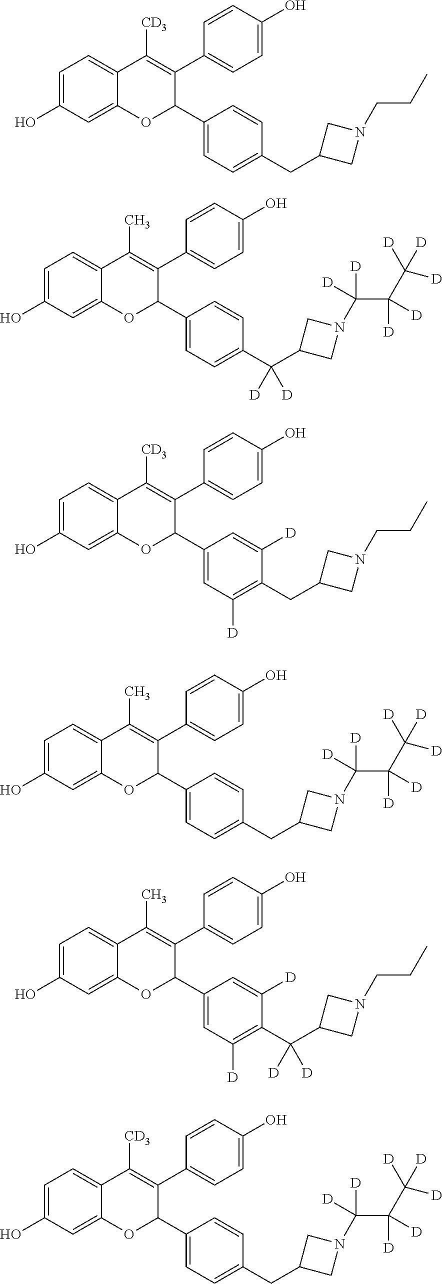

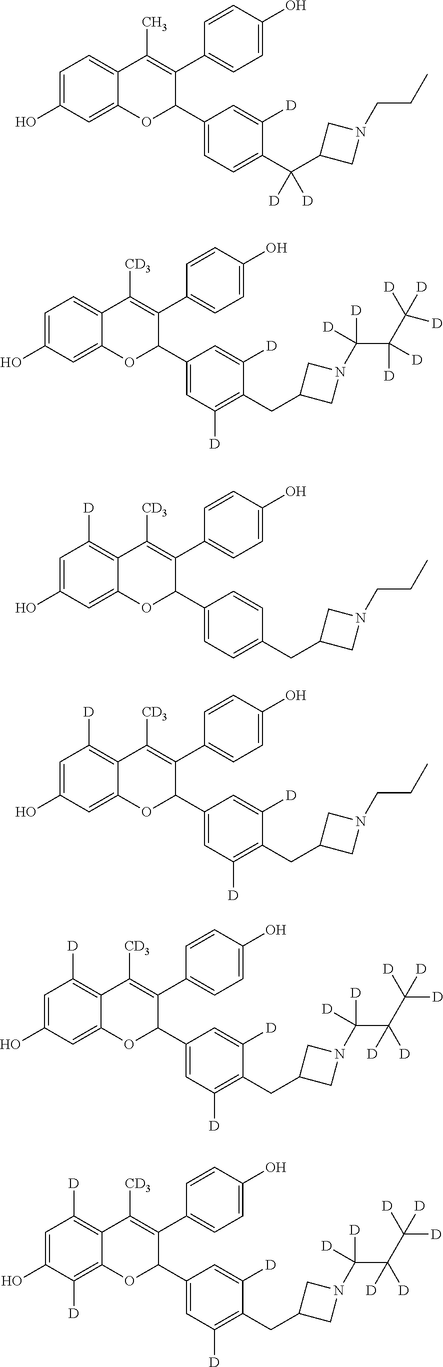

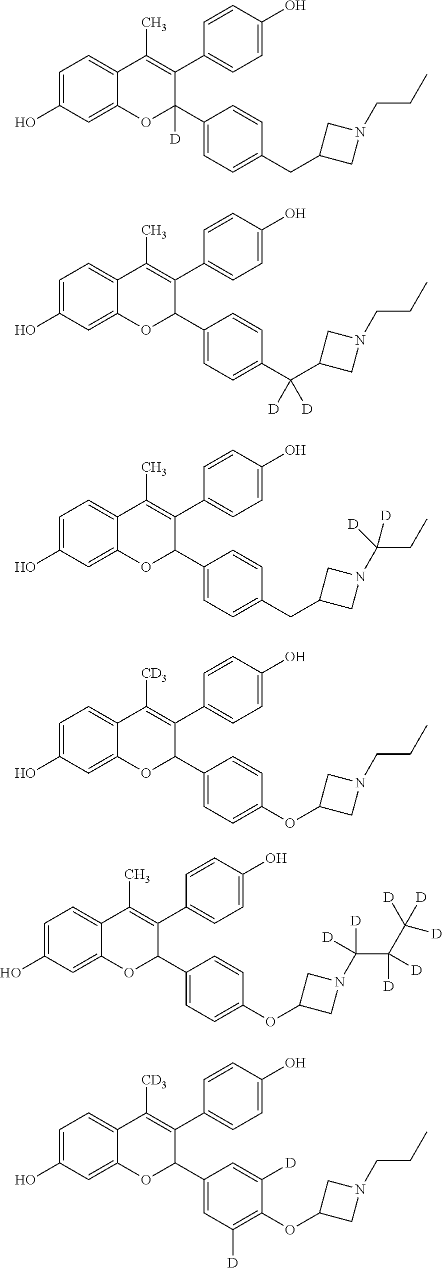

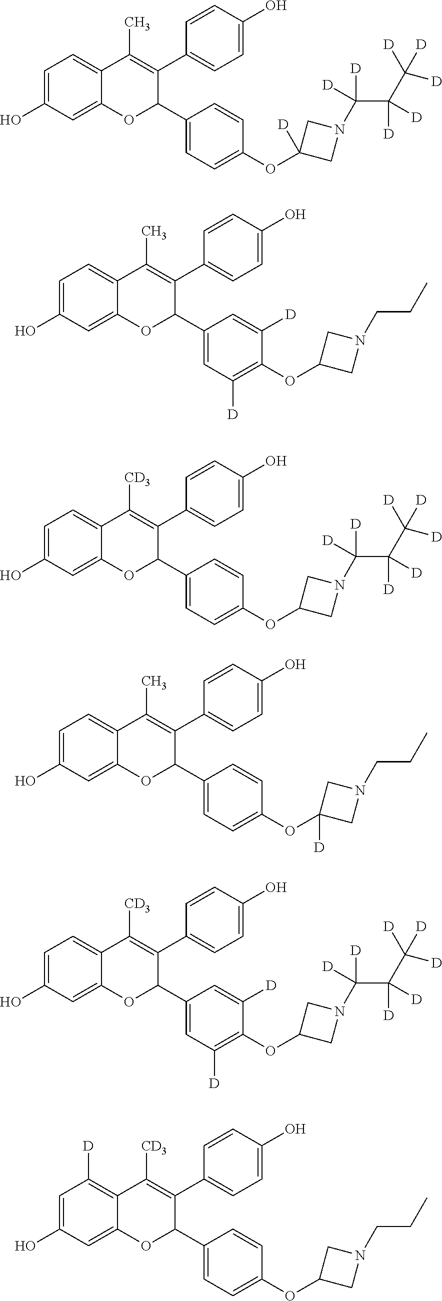

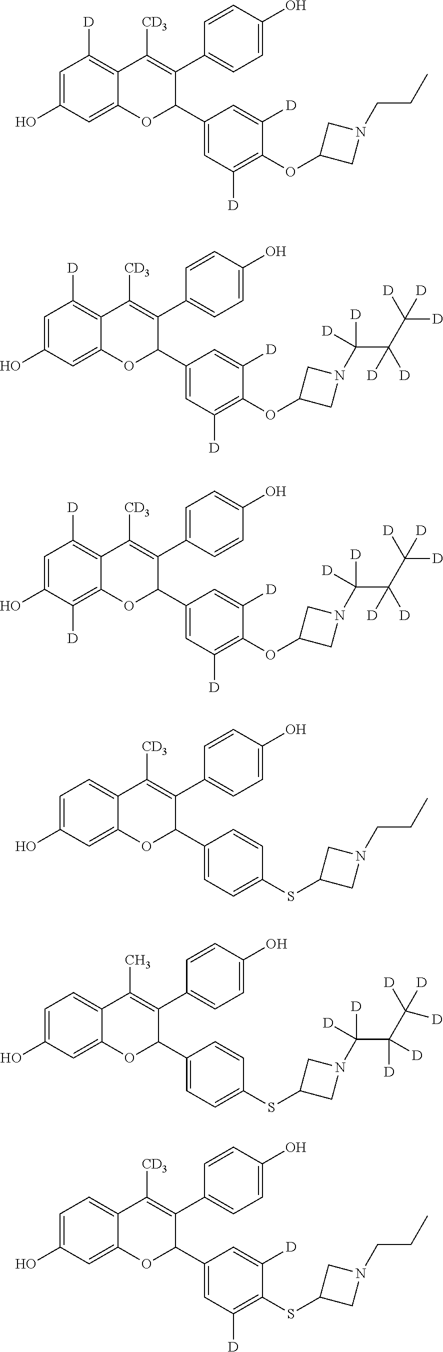

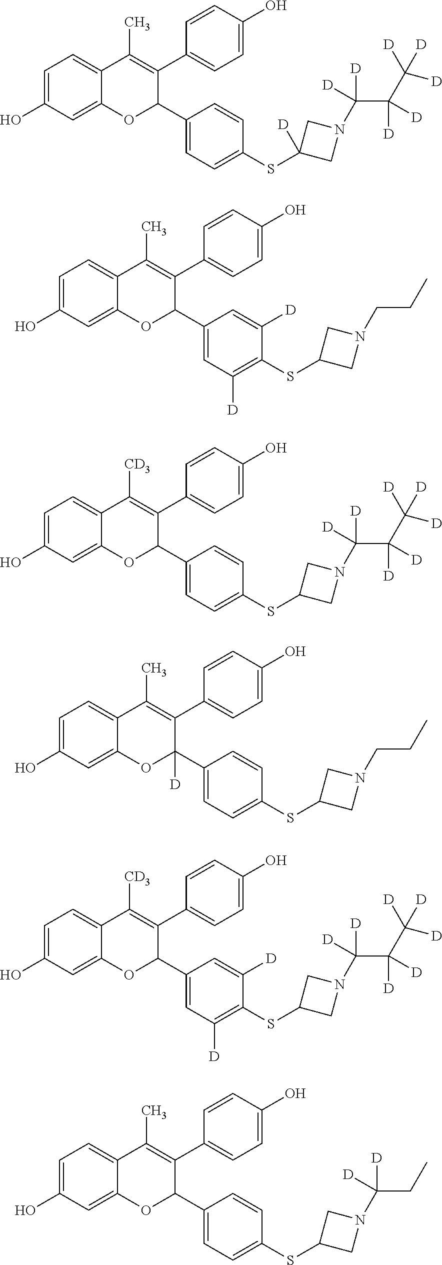

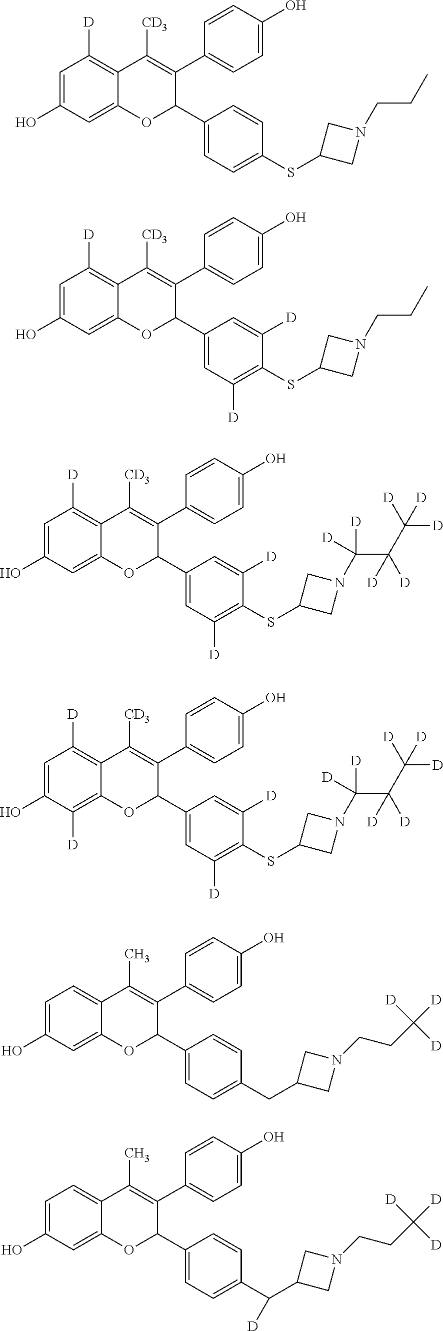

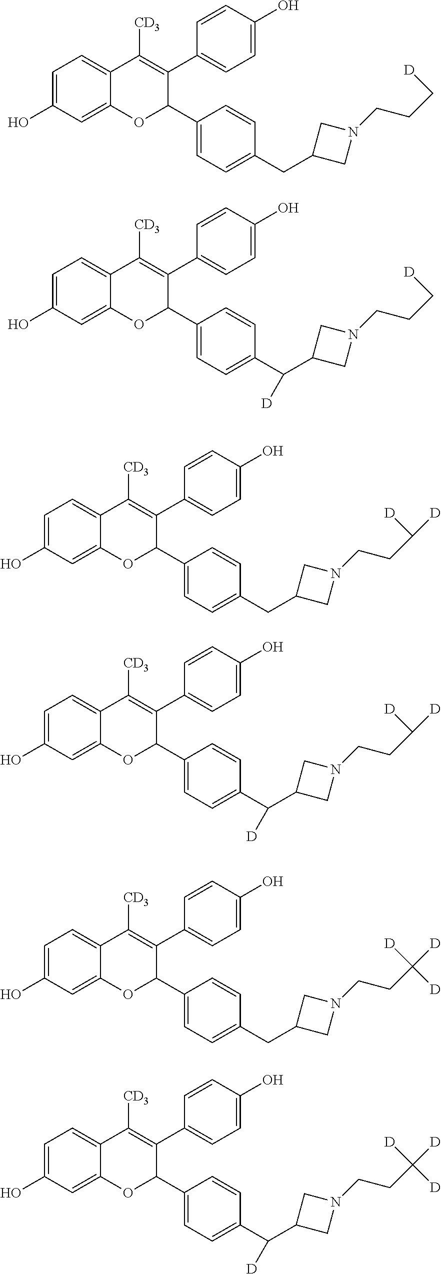

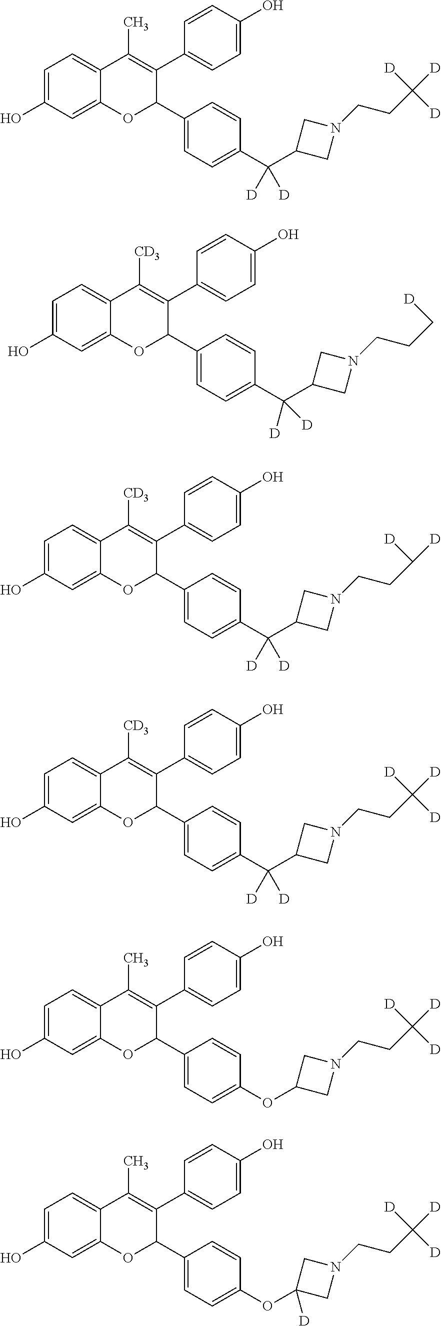

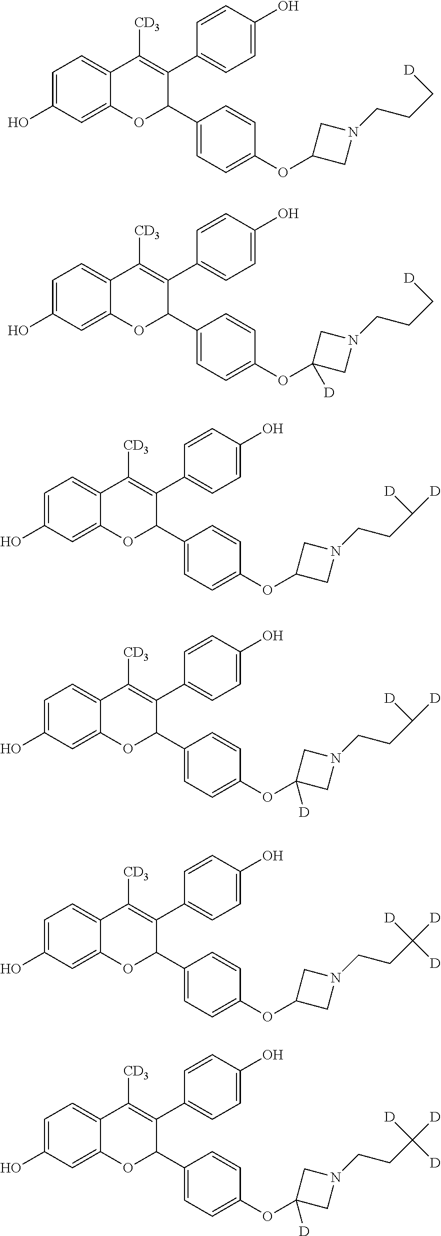

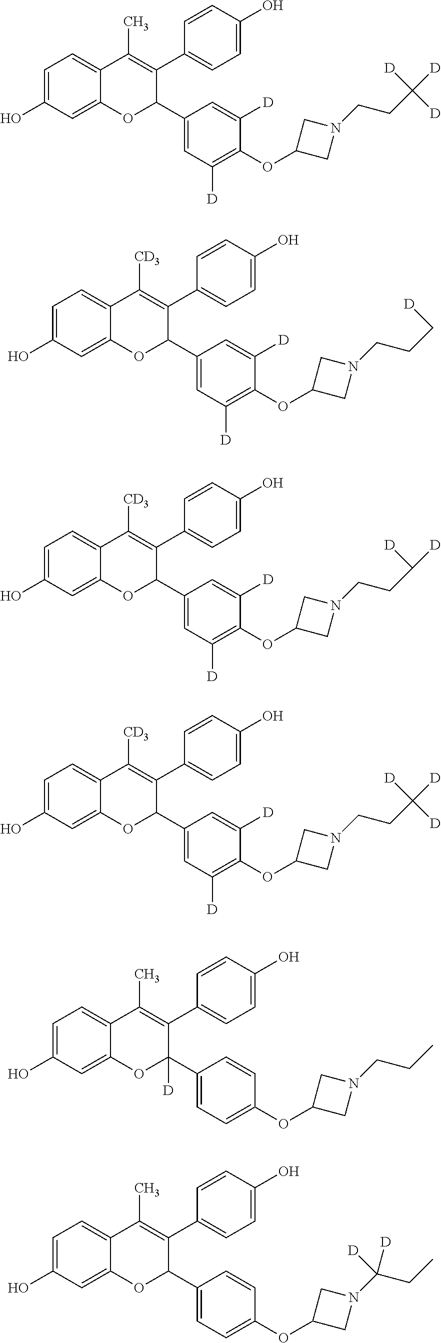

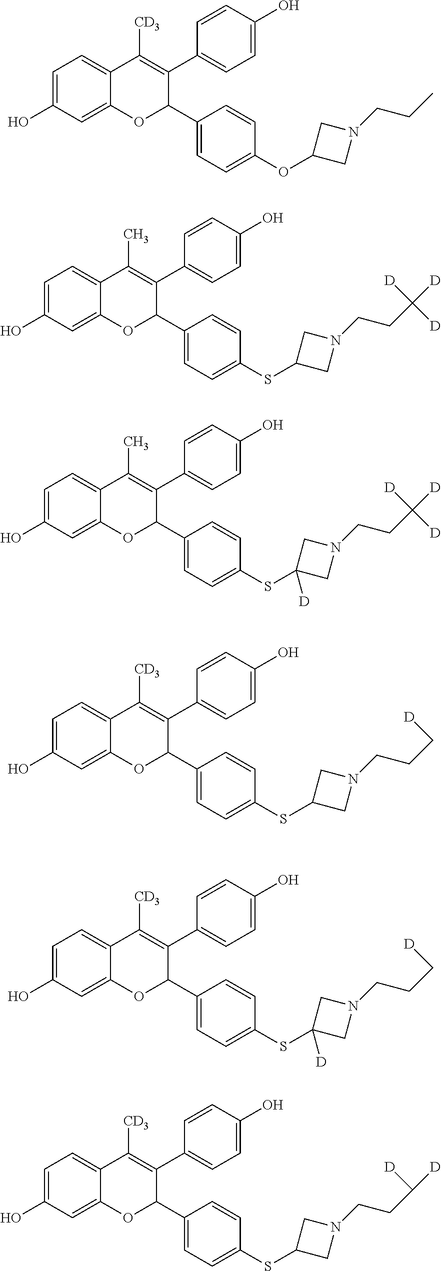 [SMILES: [2H]C([2H])([2H])C1=C(c2ccc(O)cc2)C(c2ccc(OC3CN(CCC)C3)cc2)Oc2cc(O)ccc21 ; [2H]C([2H])([2H])CCN1CC(Sc2ccc(C3Oc4cc(O)ccc4C(C)=C3c3ccc(O)cc3)cc2)C1 ; [2H]C([2H])([2H])CCN1CC([2H])(Sc2ccc(C3Oc4cc(O)ccc4C(C)=C3c3ccc(O)cc3)cc2)C1 ; [2H]CCCN1CC(Sc2ccc(C3Oc4cc(O)ccc4C(C([2H])([2H])[2H])=C3c3ccc(O)cc3)cc2)C1 ; [2H]CCCN1CC([2H])(Sc2ccc(C3Oc4cc(O)ccc4C(C([2H])([2H])[2H])=C3c3ccc(O)cc3)cc2)C1 ; [2H]C([2H])CCN1CC(Sc2ccc(C3Oc4cc(O)ccc4C(C([2H])([2H])[2H])=C3c3ccc(O)cc3)cc2)C1]

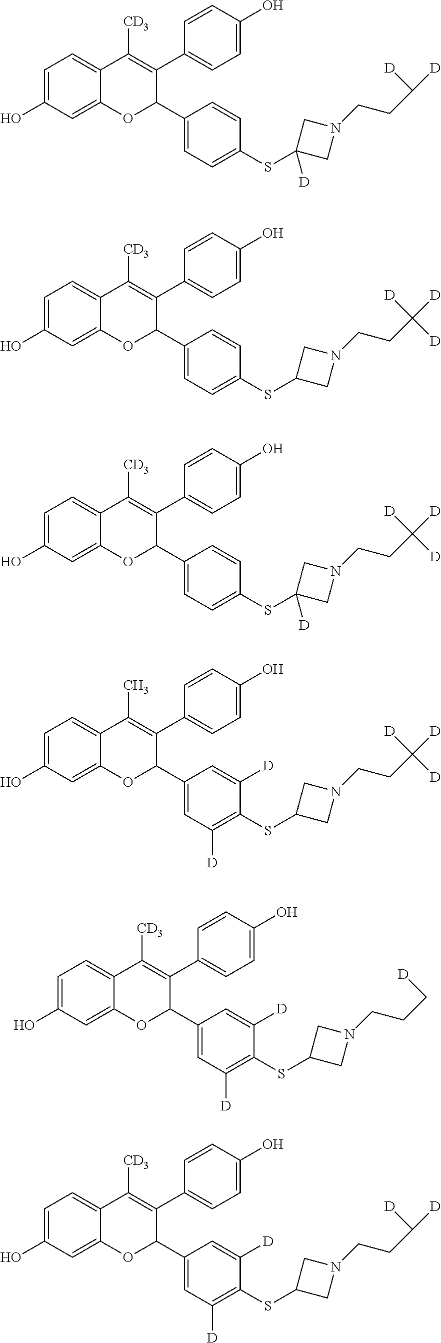

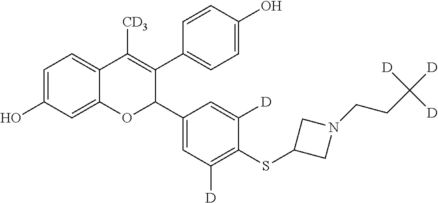 [SMILES: [2H]c1cc(C2Oc3cc(O)ccc3C(C([2H])([2H])[2H])=C2c2ccc(O)cc2)cc([2H])c1SC1CN(CCC([2H])([2H])[2H])C1]

Depending on the substituents for X, R¹ and R³, the compounds of Formula I, Formula IA, Formula II, Formula IIA and Formula III may have additional asymmetric carbon atoms, which may exist in various stereoisomeric forms. These various stereoisomeric forms are contemplated to be within the scope of the present disclosure. Compounds of the present disclosure include diastereomerically or enantiomerically pure compounds of Formula I, Formula IA, Formula II, Formula IIA and Formula III. These diastereomerically or enantiomerically pure compounds of Formula I, Formula IA, Formula II, Formula IIA and Formula III provided herein may be prepared according to techniques known to those of skill in the art. For instance, they may be prepared by chiral or asymmetric synthesis from a suitable optically pure precursor or obtained from a racemate or mixture of enantiomers or diastereomers by any conventional technique, for example, by chromatographic resolution using a chiral column, TLC or by the preparation of diastereoisomers, separation thereof and regeneration of the desired enantiomer or diastereomer. See, e.g., “Enantiomers, Racemates and Resolutions,” by J. Jacques, A. Collet, and S. H. Wilen, (Wiley-Interscience, New York, 1981); S. H. Wilen, A. Collet, and J. Jacques, Tetrahedron, 2725 (1977); E. L. Eliel Stereochemistry of Carbon Compounds (McGraw-Hill, N Y, 1962); and S. H. Wilen Tables of Resolving Agents and Optical Resolutions 268 (E. L. Eliel ed., Univ. of Notre Dame Press, Notre Dame, Ind., 1972, Stereochemistry of Organic Compounds, Ernest L. Eliel, Samuel H. Wilen and Lewis N. Manda (1994 John Wiley & Sons, Inc.), and Stereoselective Synthesis A Practical Approach, Mihály Nógrádi (1995 VCH Publishers, Inc., NY, N.Y.).

In certain embodiments, a diastereomerically pure compound of Formula I, Formula IA, Formula II, Formula IIA or Formula III may be obtained by reaction of the racemate or mix of diastereomers with a suitable optically active acid or base. Suitable acids or bases include those described in Bighley et al., 1995, Salt Forms of Drugs and Adsorption, in Encyclopedia of Pharmaceutical Technology, vol. 13, Swarbrick & Boylan, eds., Marcel Dekker, New York; ten Hoeve & H. Wynberg, 1985, Journal of Organic Chemistry 50:4508-4514; Dale & Mosher, 1973, J. Am. Chem. Soc. 95:512; and CRC Handbook of Optical Resolution via Diastereomeric Salt Formation, the contents of which are hereby incorporated by reference in their entireties.

Enantiomerically or diastereomerically pure compounds can also be recovered either from the crystallized diastereomer or from the mother liquor, depending on the solubility properties of the particular acid resolving agent employed and the particular acid enantiomer or diastereomer used. The identity and optical purity of the particular compound so recovered can be determined by polarimetry or other analytical methods known in the art. The diastereoisomers can then be separated, for example, by chromatography or fractional crystallization, and the desired enantiomer or diastereomer regenerated by treatment with an appropriate base or acid. The other enantiomer or diastereomer may be obtained from the racemate or mix of diastereomers in a similar manner or worked up from the liquors of the first separation.

In certain embodiments, an enantiomerically or diastereomerically pure compound can be separated from racemic compound or a mixture of diastereomers by chiral chromatography. Various chiral columns and eluents for use in the separation of the enantiomers or diastereomers are available and suitable conditions for the separation can be empirically determined by methods known to one of skill in the art. Exemplary chiral columns available for use in the separation of the enantiomers provided herein include, but are not limited to CHIRALPACK® IC, CHIRALCEL® OB, CHIRALCEL® OB-H, CHIRALCEL® OD, CHIRALCEL® OD-H, CHIRALCEL® OF, CHIRALCEL® OG, CHIRALCEL® OJ and CHIRALCEL® OK.

The compounds provided herein can be prepared from readily available starting materials using the following general methods and procedures. See, e.g., Synthetic Schemes below. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvent used, but such conditions can be determined by one skilled in the art by routine optimization procedures.

Additionally, as will be apparent to those skilled in the art, conventional protecting groups may be necessary to prevent certain functional groups from undergoing undesired reactions. The choice of a suitable protecting group for a particular functional group as well as suitable conditions for protection and deprotection are well known in the art. For example, numerous protecting groups, and their introduction and removal, are described in T. W.

Greene and P. G. M. Wuts, Protecting Groups in Organic Synthesis, Second Edition, Wiley, New York, 1991, and references cited therein.

The compounds provided herein may be isolated and purified by known standard procedures. Such procedures include (but are not limited to) recrystallization, column chromatography or HPLC. The following schemes are presented with details as to the preparation of representative compounds of Formula I, Formula IA, Formula II, Formula IIA and Formula III that have been listed herein. The compounds provided herein may be prepared from known or commercially available starting materials and reagents by one skilled in the art of organic synthesis.

The following non-limiting schemes for the preparation are exemplary of the methods used to prepare the compounds of Formula I, Formula IA, Formula II, Formula IIA and Formula III. General processes for preparing compounds of the instant invention are provided as further embodiments of the invention and are illustrated in the following Schemes. In the schemes, unless indicated to the contrary, R¹, R², R³, R⁴, R⁵, R⁶, R⁷, R⁸, n, p, q and X are as defined hereinabove.

The following abbreviations may be used in the Schemes and Examples below: aq. (aqueous); CSA (camphorsulfonic acid); DBU (1,8-diazabicyclo[5.4.0]undec-7-ene); DCM (dichloromethane); DEA (diethylamine); DHP (dihydropyran); DIPEA (N,N-diisopropylethylamine); DMAP (4-dimethylaminopyridine); DMSO (dimethyl sulphoxide); EA (ethyl acetate); ee (enantiomeric excess); equiv. (equivalent); ethanol (EtOH); h (hour or hours); Hex (hexanes); HPLC (high-performance liquid chromatography); IPA (isopropyl alcohol); KHMDS (potassium bis(trimethylsilyl)amide); LAH (lithium aluminum hydride); LCMS (liquid chromatography-mass spectrometry); LDA (lithium diisopropylamide); LiHMDS (lithium bis(trimethylsilyl)amide); Me (methyl); MeOH (methanol); min (minute or minutes); NMR (nuclear magnetic resonance); OBs (brosylate); OMs (mesylate); ONs (nosylate): OTs (tosylate); Pd/C (palladium on carbon); PPh₃O or Ph₃PO or OPPh₃ (triphenylphosphine oxide); Pt/C (platinum on carbon); Rf (retention factor); RT (room temperature); TEA (triethylamine); THF (tetrahydrofuran); THP (tetrahydropyran); TLC (thin layer chromatography); TsOH (p-toluenesulfonic acid or tosylic acid); and UV (ultraviolet).

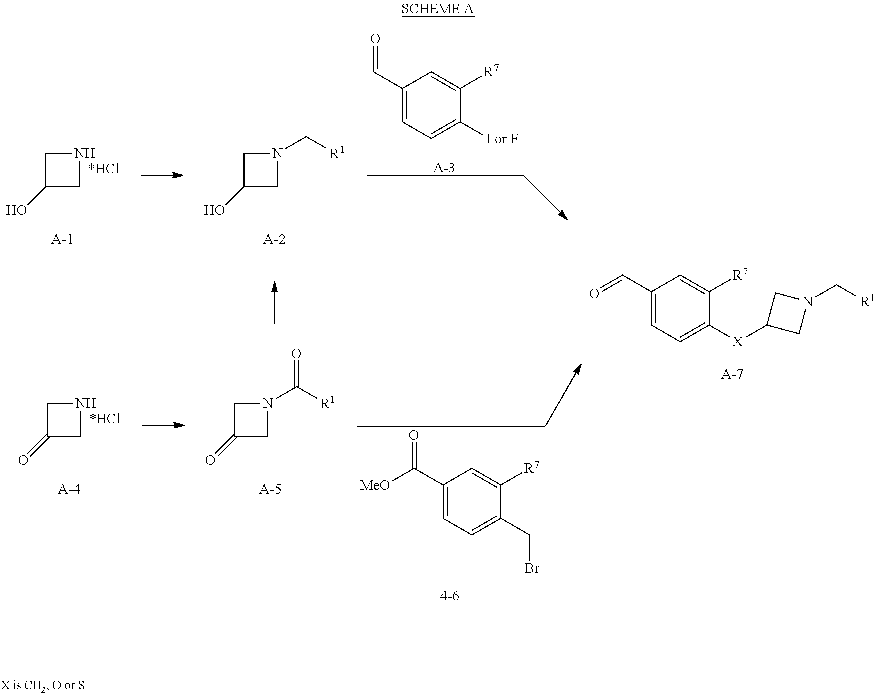

As exemplified in Scheme A, intermediate A-7 for the synthesis of compounds of Formula I, Formula II, and Formula III can be synthesized from readily available functionalized azetidines A-1 and A-4.

Compound A-2 can be prepared by direct alkylation of A-1 or its O-protected analog using a suitably functionalized alkylating agent containing R¹, such as LCH₂R¹ under amine alkylation conditions, wherein L is a leaving group, such as halide (e.g., Br, Cl, 1) or other leaving group, such as OTs, OBs, ONs, OMs, triflate, nonaflate, tresylate and the like.

Alternatively, A-2 can be prepared by reductive amination of A-1 with HC(O)R¹ in the presence of hydrogen and a hydrogenation catalyst, such as Pt, Pd and the like. Alternatively, A-2 is prepared by reacting XC(O)R¹, where X is a leaving group, with A-4 under amide forming conditions to form the amidoketone A-5 followed by reduction of the resulting amidoketone A-5 using reducing agents known in the art, such as LAH and the like.

Nucleophilic aromatic substitution by A-2 of a halide on functionalized benzaldehyde A-3 by either aryl nucleophilic substitution (for fluoro-substituted A-3) or via Ullman coupling conditions (for iodo-substituted A-3) under conditions known in the art gives rise to intermediate A-7, wherein X is O. Similarly, the corresponding intermediate A-7, where X is S, can be prepared by preparing the halides from a azetidine A-1 under nucleophilic substitution reaction conditions, using, for example, hydrochloric acid or hydrobromic acid or hydroiodic acid to form the corresponding chloride, bromide or iodide, respectively, or by reacting azetidine A-1 with an inorganic acid halide, such as SOCl₂, PCl₅, PCl₃, POCl₃ and the like, to form the corresponding chloride. The product thereof is reacted with a sulfide, such as sodium hydrogen sulfide and the like, to form the corresponding thiol. The thiol is reacted with suitably functionalized alkylating agent containing R¹, such as LCH₂R¹ under amine alkylation conditions, wherein L is a leaving group, such as halide (e.g., Br, Cl, I) or another leaving group, such as OTs, OBs, ONs, OMs, triflate, nonaflate, tresylate and the like, and the resulting product is reacted with A-3 to form the A-7, where X is S.

To form compounds where X is CH₂, amidoketone A-5 can be coupled to ester A-6 via the phosphonium salt of A-5 in a Wittig reaction under Wittig forming conditions to form the alkene A-6.1.

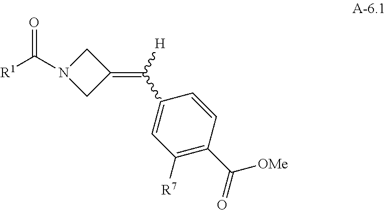

Reduction of the resulting alkene followed by reduction of the amide and ester functions under reducing conditions known in the art provides the benzylic alcohol A-6.2.

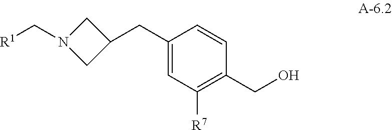

Oxidation of the benzyl alcohol using oxidizing agents known in the art such as copper chromite; DMSO; Collins' reagent; Corey's reagent; pyridinium dichromate; sodium dichromate in water; and the like or DMSO, dicyclohexylcarbodiimide and anhydrous phosphoric acid under Moffatt oxidation conditions or anhydrous phosphoric acid and oxalyl chloride under Swern oxidation conditions and the like furnishes the aldehyde A-7, wherein X is CH₂. A-7 is an intermediate in the reaction in Scheme C, hereinbelow.

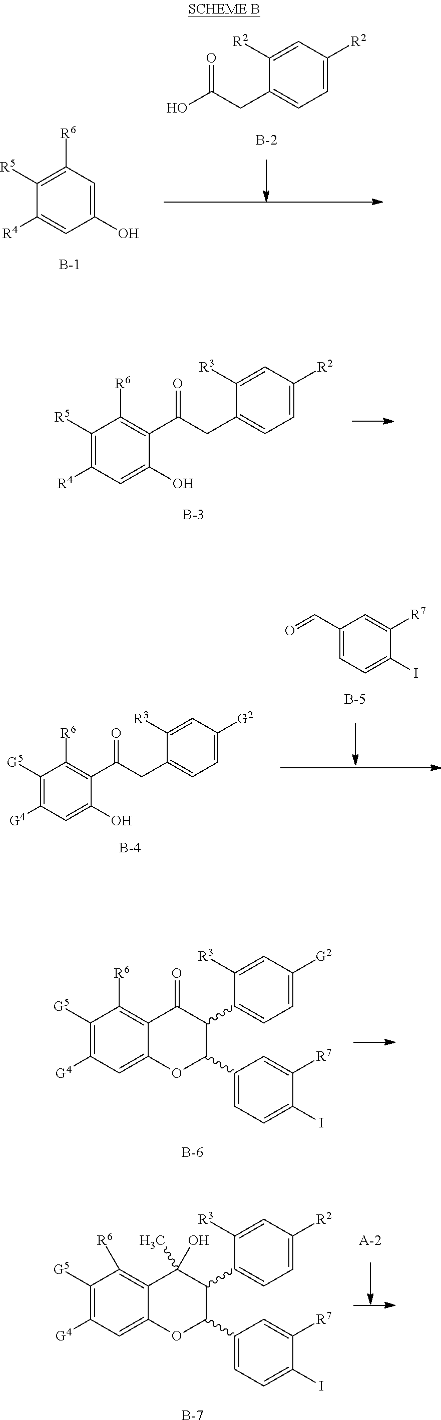

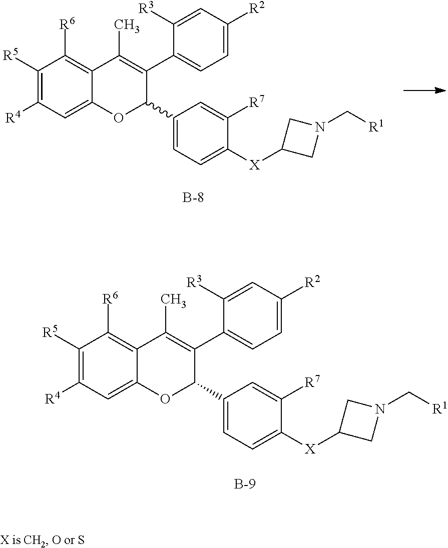

In Scheme B, G² is defined as H, halogen, cyano or an oxygen-protecting group known in the art, and one of G⁴ and G⁵ is H or halogen and the other is H, halogen or an oxygen-protecting group known in the art.

As exemplified in Scheme B, compounds of Formula I, Formula II, and Formula III can be synthesized beginning with phenol B-1. B-1 is treated with a substituted phenylacetic acid or acylating derivative thereof such as B-2 and a Lewis acid, such as AlBr₃, AlCl₃, GaCl₃, FeCl₃, BF₃, BCl₃, SbCl₅, SbCl₃, and the like under Friedel-Crafts acylation conditions to provide ketone B-3. Free phenols are then blocked with the protecting groups G², G⁴ and G⁵, where present, to afford B-4. Chromanone B-6 is produced by treatment of B-3 with an iodobenzaldehyde B-5 under basic conditions using weak bases known in the art for such coupling reaction, such as piperidine, or DBU or similar bases in butanol or similar appropriate solvent. The ketone moiety B-6 is converted to a tertiary alcohol by treatment with a source of methyl anion, such as treatment with methyl magnesium bromide or similar source followed by deprotection of G², if G² is an oxygen protecting group, to give B-7. The azetidine side chain is then introduced by treatment of B-7 with A-2 or the corresponding thiol under Ullmann reaction conditions using copper as a catalyst. Elimination of tertiary alcohol using acidic conditions known in the art and deprotection of the protecting group thereon then furnishes B-8 as a mixture of enantiomers or diastereomers. Resolution of the structural isomers via, for example, chiral chromatography or crystallographic resolution gives B-9.

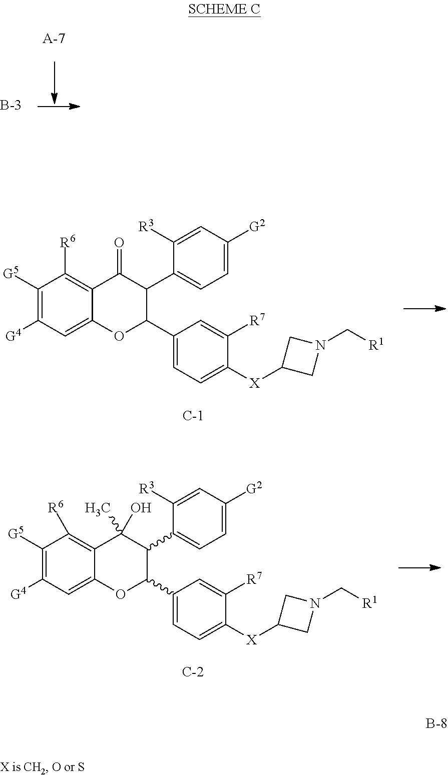

In Scheme C, G² is defined as H, halogen, cyano or an oxygen-protecting group known in the art, and one of G⁴ and G⁵ is H or halogen and the other is H, halogen or an oxygen-protecting group known in the art.

As exemplified in Scheme C, compounds of Formula I, Formula II, and Formula III may also be synthesized from compound B-3 by condensation with aldehyde A-7 under basic conditions such as piperidine, or DBU or similar bases in butanol or similar solvent to furnish C-1. The ketone moiety of C-1 is then converted to a tertiary alcohol by treatment with a source of methyl anion such as treatment with methyl magnesium bromide or similar source to give C-2. Elimination of the tertiary alcohol using acidic conditions known in the art and deprotection of protecting groups thereon then furnish previously described compound B-8 as a mixture of enantiomers.

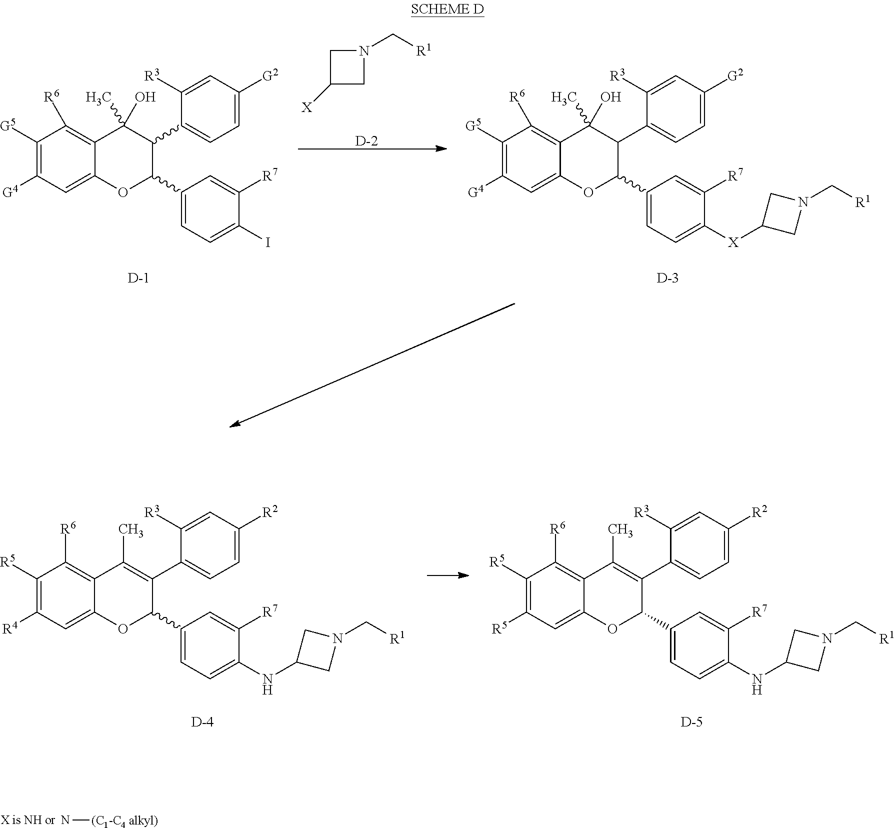

In Scheme D, G² is defined as H, halogen, cyano or an oxygen-protecting group known in the art, and one of G⁴ and G⁵ is H or halogen and the other is H, halogen or an oxygen-protecting group known in the art.

As exemplified in Scheme D, compounds of Formula I, Formula II, and Formula III where X is NH or N—(C₁-C₄ alkyl) can be synthesized beginning with iodide D-1. D-1 is treated with a 3-amino substituted azetidine derivative such as D-2 (in which X is NH or N—(C₁-C₄ alkyl)) under Ullmann type coupling conditions using a copper-based catalyst and a ligand such as 1,10-phenathroline in a solvent such as NMP or DMF to afford D-3. Elimination of the tertiary alcohol in D-3 using acidic conditions known in the art then furnishes D-4 as a mixture of enantiomers or diastereomers. Resolution of the structural isomers via, for example, chiral chromatography or crystallographic resolution affords D-5.

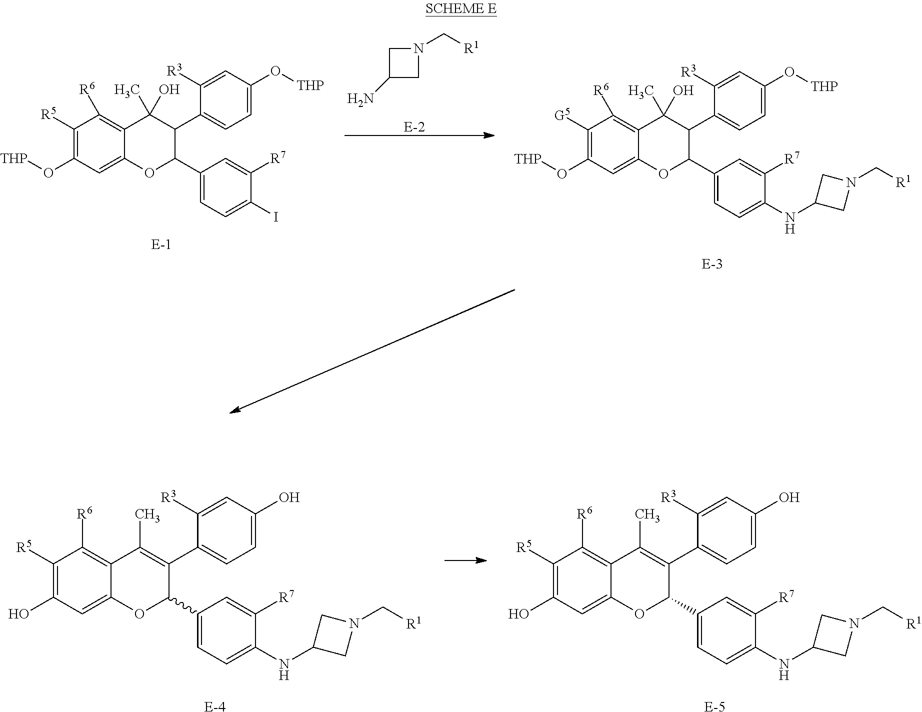

Scheme E exemplifies the preparation of compounds of the subject application where X is NH. Intermediate E-1 is dissolved in butyronitrile (0.2 M). To this solution are added N-propylazetidine-3-amine (3 equiv.)(E-2), cesium carbonate (2.0 equiv.), and copper iodide (0.5 equiv.). The solution is degassed by passing a stream of nitrogen gas through it. The mixture is heated at 125° C. for 0.5 to 2 h under inert atmosphere. The mixture is allowed to cool to RT. Ethyl acetate is added and insoluble materials removed by filtration through a pad of Celite®. The resulting solution is concentrated in vacuo and the crude product E-3 purified via silica gel chromatography. Intermediate E-3 is dissolved in acetic acid (0.1 M) and heated at 90° C. for 2-4 h. The mixture is concentrated in vacuo to afford crude product. This material is purified by silica gel chromatography to afford the desired product E-4 among other materials. The enantiomers are separated via chiral chromatography to provide E-5.

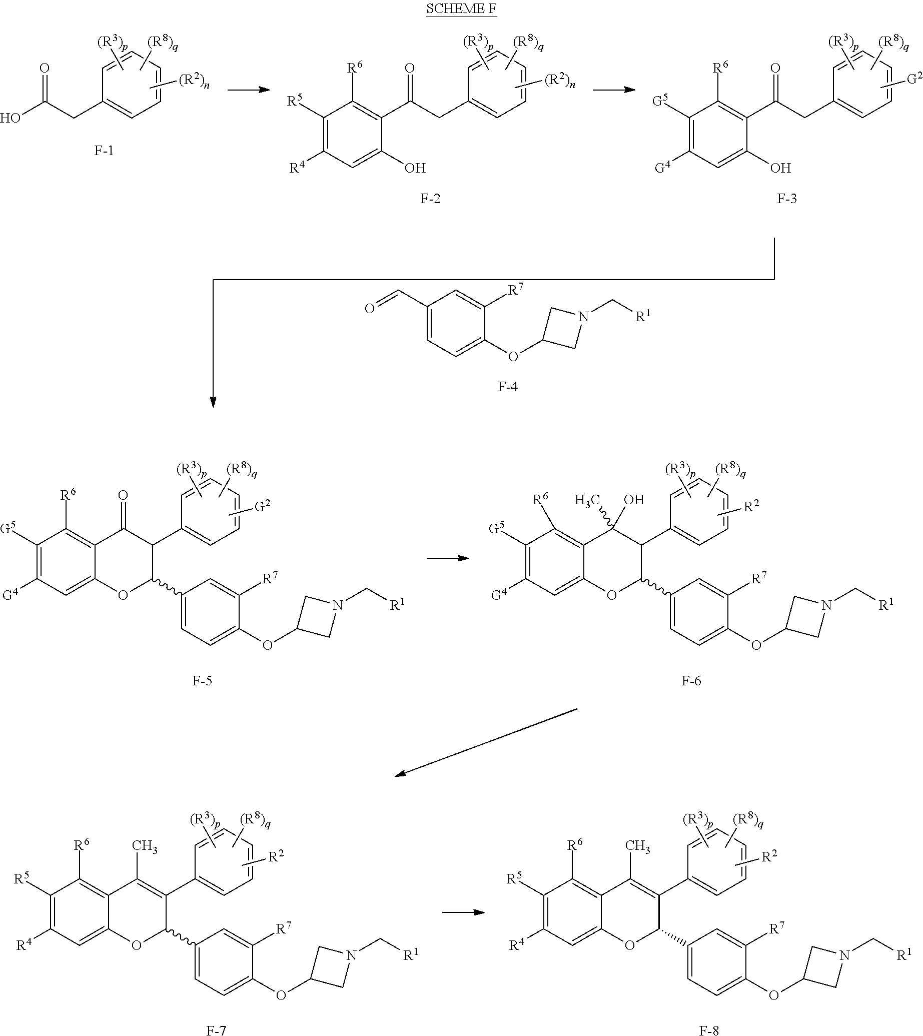

In Scheme F, G² is defined as H, halogen, cyano or an oxygen-protecting group known in the art, and one of G⁴ and G⁵ is H or halogen and the other is H, halogen or an oxygen-protecting group known in the art.

As exemplified in Scheme F, compounds of Formula I and Formula IA of the present invention wherein X is O can be synthesized beginning with substituted phenylacetic acid derivative, F-1. F-1 is treated with a substituted phenol derivative and a Lewis acid, such as AlBr₃, AlCl₃, GaCl₃, FeCl₃, BF₃, BCl₃, SbCl₅, SbCl₃, and the like under Friedel-Crafts acylation conditions to provide ketone F-2. Free phenols are then blocked with the protecting groups G², G⁴ and G⁵, where present, to afford F-3. Chromanone F-5 is produced by treatment of F-3 with an 4-substituted benzaldehyde derivative F-4 (prepared by a Mitsunobu reaction as shown for A-7 where X═O in Scheme A) under basic conditions using weak bases known in the art for such coupling reaction, such as piperidine, or DBU or similar bases in butanol or similar appropriate solvent. The ketone moiety F-5 is converted to a tertiary alcohol by treatment with a source of methyl anion, such as treatment with methyl magnesium bromide or similar source followed by deprotection of G², if G² is an oxygen protecting group, to give F-6. Elimination of tertiary alcohol using acidic conditions known in the art and deprotection of the protecting group thereon then furnishes F-7 as a mixture of enantiomers or diastereomers. Resolution of the structural isomers via, for example, chiral chromatography or crystallographic resolution affords F-8.

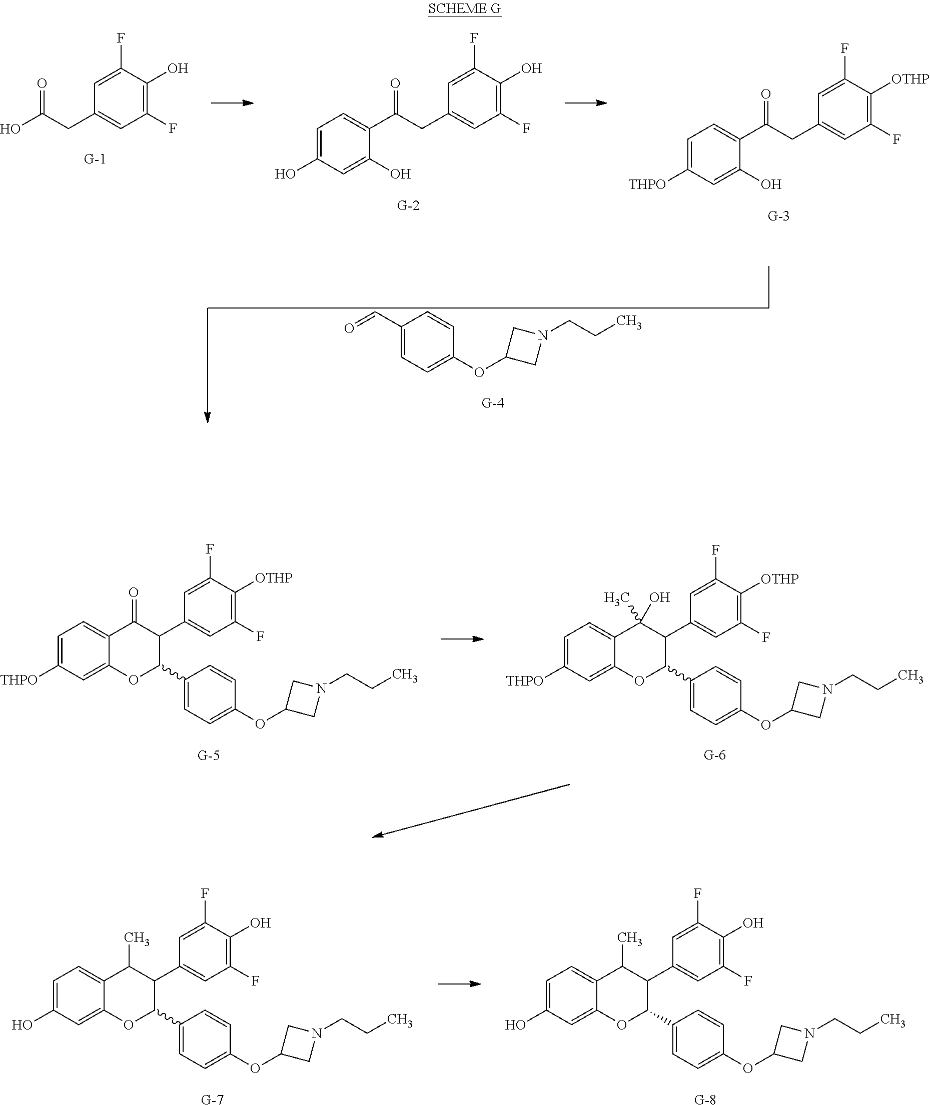

Scheme G illustrates how to prepare a compound of Formula I and Formula IA of the present invention wherein X is O. A solution of 4-hydroxy-3,5-difluorophenylacetic acid (1 equiv.) and resorcinol (1.1 equiv.) in toluene (2 M) is purged with nitrogen and treated with boron trifluoride etherate (3 equiv.) added via addition funnel. The resulting mixture is heated at 90° C. until all solids dissolved (1-5 h). The mixture is then cooled to RT and quenched by slow addition of a saturated solution of sodium acetate. The product is isolated as a solid or oil and may be used as is or purified with silica gel chromatography. Intermediate G-2 is dissolved in ethyl acetate (1.2 M) and treated with p-toluenesulfonic acid (0.001-0.01 equiv.). To this solution is added dropwise to avoid overheating dihydropyran (4 equiv.). The resulting mixture is stirred at RT for 12-36 h. A suspension may form. The product may be isolated via filtration or by aqueous work up. The reaction mixture is quenched with saturated sodium bicarbonate solution. The organic layer is separated. The aqueous layer is extracted with ethyl acetate. The combined organic phases are dried over anhydrous sodium sulfate, filtered, and concentrated. The product is then purified via silica gel chromatography to furnish G-3. Condensation of G-3 with aldehyde G-4 is achieved by dissolving these materials in butanol (0.5 M) and treating the resulting solution with piperidine (0.3 equiv.) and diazabicyclo[5.4.0]undec-7-ene (0.3 equiv.). The mixture is brought to reflux and water removed using a Dean-Stark apparatus. After removal of ca. one half of the starting butanol, the mixture is heated at reflux for an additional 1-4 h. The mixture is then allowed to cool to RT and 2-propanol (half the volume of butanol used) is added. A solid or gum may form. Alternatively, solvent and volatile materials are removed under reduced pressure to afford a gum. This material is purified via silica gel chromatography to afford the desired product G-5 among other products. Ketone G-5 is dissolved in tetrahydrofuran (0.3 M). The solution is cooled to 0° C. in an ice-bath. Methyl magnesium chloride (1.8 equiv.) is then added slowly to maintain a reaction temperature below 5° C. via syringe. After the addition is complete, the mixture is allowed to warm to RT and the reaction quenched by slow addition of saturated ammonium chloride. The product may precipitate as a gum or solid. Alternatively, the product may be isolated by extraction with ethyl acetate or other solvent. The combined organic phases are dried over anhydrous sodium sulfate, concentrated and purified via silica gel chromatography to afford the desired product. Tertiary alcohol G-6 is dissolved in acetic acid (1 M) and heated at 90° C. for 1-5 h. The reaction mixture is then cooled to RT and concentrated in vacuo. The resulting oil is purified via silica gel chromatography to afford chromene G-7. The enantiomers of G-7 are separated via chiral chromatography to furnish the desired chromene G-8.

Additional embodiments within the scope provided herein are set forth in non-limiting fashion elsewhere herein and in the examples hereinbelow. It should be understood that these examples are for illustrative purposes only and are not to be construed as limiting in any manner.

The following non-limiting preparations and examples are illustrative of the present disclosure:

### EXAMPLES

### Preparation 1: Preparation of 1-propionylazetidin-3-one

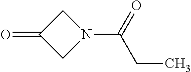

3-Azetidinone hydrochloride (10.000 g, 93.0 mmol, 1.0 equiv.), anhydrous 1,2-dichloroethane (200 mL) and diisopropylethylamine (38.9 mL, 223 mmol, 2.4 equiv.) were added to a round bottom flask (500 mL) to provide a light yellow suspension. The suspension was sonicated for 1 h and then cooled to −10° C. (dry ice/MeOH) for 10 min. Propionyl chloride (9.8 mL, 112 mmol, 1.2 equiv.) was added dropwise to the cooled suspension to provide an orange solution. The reaction was removed from the bath and stirred at RT for 16 h. The solvent was removed to provide a semi-solid. The semi-solid was suspended into EA (300 mL) and the suspension was filtered. The solid was rinsed with EA (2×100 mL). TLC analysis (10% MeOH/DCM, KMnO₄ stain/Heat) indicated there were three spots: R_{f}: 0.2, 0.5, 0.7. TLC (50% EA/Hex, KMnO₄ stain/Heat) indicated there were two spots: R_{f}: 1, 0.3. The filtrate was concentrated and adsorbed onto silica gel (25 g) and chromatographed through silica gel (100 g cartridge) with DCM (5 min) then 0-10% MeOH over 15 min. Product came off early from the column in DCM and continued to elute from the column up to 10% MeOH. TLC in both solvent systems was carried out to determine if any propionyl chloride was present in early fractions. Fractions containing product were pooled and concentrated to afford the title compound as a yellow liquid (11.610 g, 98.2%).

¹H NMR (300 MHz, CDCl₃) δ 4.80 (d, J=5.6 Hz, 4H), 2.29 (q, J=7.5 Hz, 2H), 2.01 (s, 3H), 1.18 (t, J=7.5 Hz, 3H).

### Preparation 2: Preparation of 1-propylazetidin-3-ol

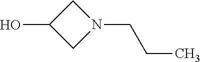

Lithium aluminum hydride (10.397 g, 273.9 mmol, 3.0 equiv.) was suspended into THF (200 mL) and cooled in an ice-bath. A solution of the product of Preparation 1, 1-propionylazetidin-3-one (11.610 g, 91.3 mmol, 1.0 equiv.), in 100 mL of THF was added dropwise to the reaction mixture via a pressure equalizing addition funnel over 30 min. The addition funnel was removed. The flask was then fitted with a condenser and the reaction was heated at reflux in an oil bath at 75° C. for 16 h. The reaction was cooled in an ice-bath for 20 min and sodium sulfate decahydrate (Glauber's salt, 25 g) was added in small portions over 20 min. After complete addition, the mixture was stirred at RT for 2 h. The mixture was filtered through a bed of Celite® (2 cm) and the solids rinsed with EA (2×250 mL). The clear solution was concentrated to a pale yellow liquid (9.580 g, 91.1%). NMR indicated the presence of THF and EA. This material was used without further purification in the preparation of the compounds of the examples below.

¹H NMR (300 MHz, CDCl₃) δ 4.39 (pent, J=6 Hz, 1H), 3.62-3.56 (m, 2H), 2.90-2.85 (m, 2H), 2.41 (t, J=7.5 Hz, 2H), 1.34 (hextet, J=7.2 Hz, 2H), 0.87 (t, J=7.8 Hz, 3H).

### Preparation 3: Preparation of 1-(3,3,3-trifluoropropyl)azetidin-3-ol

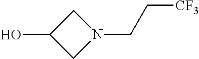 [SMILES: OC1CN(CCC(F)(F)F)C1]

### Step 1. Preparation of 3-((tert-butyldimethylsilyl)oxy)azetidine

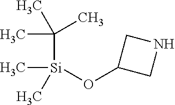

To a stirred solution of 1-benzhydrylazetidin-3-ol (20.000 g, 83.6 mmol, 1.0 equiv.) and tert-butylchlorodimethylsilane (15.115 g, 100.3 mmol, 1.2 equiv.) in DCM (300 mL) at RT was added imidazole (6.827 g, 100.3 mmol, 1.2 equiv.). After 3 h, the reaction mixture was filtered, and the filter cake was washed with DCM (50 mL). The filtrate was concentrated to dryness under reduced pressure. The residue was adsorbed onto silica gel (35 g) and chromatographed through silica gel (100 g cartridge) with 0-10% EA/Hex to two UV active portions (both product). The excess tert-butylchlorodimethylsilane came off the column after the product. Fractions were pooled to provide the silyl ether as a white solid (29.5 g, 99.8%). TLC: 10% EA/Hex, R_{f}: 0.7 (Product) LCMS: 354.

¹H NMR (300 MHz, CDCl₃) δ 7.40-7.17 (m, 10H), 4.51 (pent, J=6.3 Hz, 1H), 4.35 (s, 1H), 3.54-3.50 (m, 2H), 2.84-2.79 (m, 2H), 0.85 (s, 9H), 0.01 (s, 6H).

The product was dissolved into MeOH (500 mL). The solution was degassed by vacuum and the flask flushed with nitrogen. 10% o Pd/C (1.500 g, 14.1 mmol, 0.2 equiv.) was added in one portion. The suspension was degassed by vacuum and flushed with hydrogen (3 cycles) and left to stir for 72 h. LCMS indicated that the reaction was complete and the suspension was filtered through Celite® and the solid washed with MeOH (50 mL). The filtrate was concentrated to a liquid and chromatographed through silica gel (120 g cartridge) with 0-10% MeOH/DCM as eluent to afford the title compound as a pale yellow oil (9.080 g, 58%).

¹H NMR (300 MHz, CDCl₃) δ 4.6 (pent, J=6.6 Hz, 1H), 3.63-3.50 (m, 4H), 2.14 (brs, 1H), 0.85 (s, 9H), 0.02 (s, 6H).

### Step 2: Preparation of 3-((tert-butyldimethylsilyl)oxy)-1-(3,3,3-trifluoropoyl)azetidine

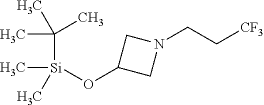 [SMILES: CC(C)(C)[Si](C)(C)OC1CN(CCC(F)(F)F)C1]

The suspension of 3-((tert-butyldimethylsilyl)oxy)azetidine (2.655 g, 14.2 mmol, 1.1 equiv.), 3-bromo-1,1,1-trifluoropropane (2.280 g, 12.9 mmol, 1.0 equiv.) and potassium carbonate (5.132 g, 37.1 mmol, 2.9 equiv.) in acetonitrile (50 mL) was heated at reflux for 12 h. TLC (20% EA/Hex, KMnO₄ stain and heat) indicated that the reaction was complete. The reaction mixture was filtered through a Celite® pad (rinsed with EA). The filtrate was concentrated and the resulting residue was loaded to a silica gel column (40 g, 0-50% EA/Hex, UV 220 nm). Fractions containing product were collected and concentrated to afford the title compound as an oil (1.82 g, 49%).

¹H NMR (300 MHz, CDCl₃) δ 4.45-4.40 (m, 1H), 3.70 (t, J=4.7 Hz, 2H), 2.85 (t, J=4.7 Hz, 2H) 2.71 (t, J=5.9 Hz, 2H), 2.23-2.14 (m, 2H), 0.89 (s, 9H), 0.05 (s, 6H).

### Step 3: Preparation of 1-(3,3,3-trifluoropropyl)azetidin-3-ol

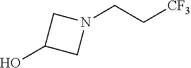 [SMILES: OC1CN(CCC(F)(F)F)C1]

A solution of 3-((tert-butyldimethylsilyl)oxy)-1-(3,3,3-trifluoropropyl)azetidine (1.82 g, 6.4 mmol, 1.0 equiv.) in MeOH (10.0 mL) was cooled to 0° C. Acetyl chloride (100 μL, 1.4 mmol, 0.2 equiv.) was added in one portion. The mixture was stirred at the same temperature for 10 min and then the ice-bath was removed. The mixture was stirred at RT for 1 h. LCMS indicated there was mostly starting material. Concentrated hydrochloric acid (1 mL) was added to the mixture and stirred at RT for 2 h. LCMS indicated that product had formed but the reaction was incomplete. The mixture was heated at 50° C. for 2 h. LCMS analysis indicated that the reaction was incomplete. Additional concentrated hydrochloric acid (2 mL) was added to the reaction and heated at 50° C. for 5 h. The reaction still showed that some starting material was present. The reaction was cooled to RT and concentrated in vacuo at 60° C. The resulting yellow oil was triturated with acetonitrile several times, then dried under high vacuum to provide a white solid (1.2 g, 91%) which was used directly next step.

¹H NMR (300 MHz, CDCl₃) δ 4.66-4.62 (m, 1H), 4.41-4.36 (m, 2H), 3.86-3.80 (m, 2H), 3.39-3.34 (m, 2H), 2.65-2.53 (m, 2H).

### Preparation 4: Preparation of 4-((1-propylazetidin-3-yl)methyl)benzaldehyde

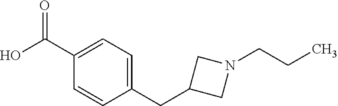 [SMILES: CCCN1CC(Cc2ccc(C(=O)O)cc2)C1]

### Step 1: Preparation of 4-(methoxycarbonyl)benzyl)triphenylphosphonium bromide

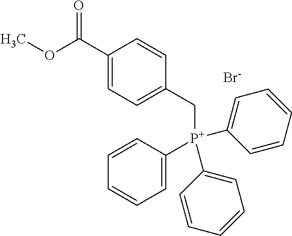 [SMILES: COC(=O)c1ccc(C[P+](c2ccccc2)(c2ccccc2)c2ccccc2)cc1 ; [Br-]]

A solution of 98.0% methyl 4-(bromomethyl)benzoate (25.550 g, 109.3 mmol, 1.0 equiv.) and triphenylphosphine (31.537 g, 120.2 mmol, 1.1 equiv.) in toluene (300 mL) was refluxed for 3.5 h, then cooled to RT. The precipitate was filtered and the white solid was washed with toluene. The resulting solid was dried under vacuum overnight to afford the title compound as a white powder (53.5 g, 99.6%).

LCMS: [M-Br]⁺, 411.

¹H NMR (300 MHz, CDCl₃) δ 7.81-7.68 (m, 11H), 7.61-7.59 (m, 6H), 7.26-7.21 (m, 2H), 5.69 (d, J=7.7 Hz, 2H), 3.85 (s, 3H).

### Step 2: Preparation of methyl 4-((1-propionylazetidin-3-ylidene)methyl)benzoate

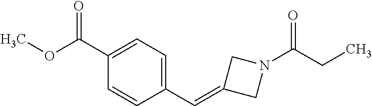 [SMILES: CCC(=O)N1CC(=Cc2ccc(C(=O)OC)cc2)C1]

Potassium tert-butoxide (10.437 g, 93.0 mmol, 1.1 equiv.) was added to a solution of (4-(methoxycarbonyl)benzyl)triphenylphosphonium bromide (45.699 g, 93.0 mmol, 1.1 equiv.) in anhydrous DMSO (200 mL). The mixture was stirred at RT for 10 min (the orange solution became an orange suspension). A solution of the product of Preparation 1, 1-propionylazetidin-3-one (10.750 g, 84.6 mmol, 1.0 equiv.), in anhydrous DMSO (100 mL) was added to the orange suspension to provide a solution. The reaction mixture was heated at 60° C. for 4 h. LCMS indicated the desired mass of product was present. The mixture was cooled to RT and poured onto ice water (600 mL) and extracted with EA (4×250 mL). The combined organic extracts were washed with brine, dried over anhydrous sodium sulfate, filtered and concentrated. The resulting residue was mixed with Hex/Ether (1:1, 300 mL) and stirred at RT for 1 h to afford the title compound mixed with Ph₃PO (˜1:1) (32 g). This mixture was used directly in the next step.

LCMS: [M+1]⁺, 260.6.

¹H NMR (300 MHz, CDCl₃) δ 8.01 (d, J=8.1 Hz, 2H), 7.70-7.43 (m, 21H OPPh₃ ratio about 1:1), 7.17 (t, J=7.1 Hz, 2H), 6.39 (d, J=10.5 Hz, 1H), 5.02-4.72 (m, 4H), 3.91 (s, 3H), 2.27-2.16 (m, 2H), 1.17 (dd, J=8.4, 1.8 Hz, 3H).

### Step 3: Preparation of methyl 4-((1-propionylazetidin-3-yl)methyl)benzoate

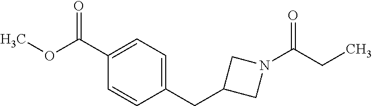 [SMILES: CCC(=O)N1CC(Cc2ccc(C(=O)OC)cc2)C1]

10% Pd/C (3.00 g, 2.8 mmol) was added to a solution of methyl 4-((1-propionylazetidin-3-ylidene)methyl)benzoate (15.000 g, 57.8 mmol, 1.0 equiv.) (containing triphenylphosphine oxide, total amount was 32 g) in MeOH (500 mL). The mixture was evacuated and blanketed with nitrogen (2 times). The reaction was evacuated and reduced with a hydrogen balloon. The reaction was stirred at RT for 3 days. TLC (50% EA/Hex) indicated the reaction was complete. LCMS indicated the desired product had formed. The suspension was filtered through a pad of Celite® and the solid rinsed with MeOH. The filtrate was concentrated and resulting residue was suspended in 20% diethyl ether/Hex (200 mL) and sonicated. Both solid and solution contained product and triphenylphosphine oxide. The mixture was concentrated and dissolved with DCM and loaded onto a silica gel column (120 g, 50%-100% EA/Hex) to afford the title compound as a light yellow oil (9.65 g, 63.8%). NMR indicated that the product contained a small amount of residual Ph₃PO.

LCMS: [M+1]⁺, 262.9.

¹H NMR (300 MHz, CDCl₃) δ 7.97 (d, J=8.4 Hz, 2H), 7.21 (d, J=8.4 Hz, 2H), 4.19-4.05 (m, 2H), 3.91 (s, 3H), 3.80-3.70 (m, 2H), 3.02-2.88 (m, 3H), 2.08 (q, J=7.7 Hz, 2H), 1.11 (t, J=7.7 Hz, 3H).

### Step 4: Preparation of (4-((1-propylazetidin-3-yl)methyl)phenyl)methanol

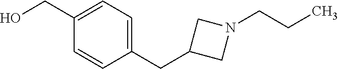 [SMILES: CCCN1CC(Cc2ccc(CO)cc2)C1]

Lithium aluminum hydride (5.20 g, 140.7 mmol, 3.8 equiv.) was added to an ice cooled THF solution of methyl 4-((1-propionylazetidin-3-yl)methyl)benzoate (9.650 g, 36.9 mmol, 1.0 equiv.) in small portions. The mixture was stirred at 0° C. for 10 min, then the mixture was evacuated and blanketed with nitrogen. The greenish suspension was heated at 66° C. for 24 h. An aliquot was taken and worked up. NMR analysis of the aliquot indicated that the reaction was complete. The reaction mixture was cooled to 0° C. and quenched with sodium sulfate decahydrate. The mixture was allowed to warm to RT and stirred for 10 min before being filtered through a pad of Celite®. After rinsing the Celite® pad with EA three times, the filtrate was concentrated to afford the title compound as a light yellow oil (7.18 g). The Celite® pad was rinsed with MeOH and the filtrate was concentrated. The residue was suspended in EA and filtered through a Celite® pad and rinsed with EA. The filtrate was concentrated to afford the title compound as a light yellow oil (0.87 g). The total yield was 8.05 g (99.4%). The material was used directly in the next step.

¹H NMR (300 MHz, CDCl₃) δ 7.27 (d, J=8.4 Hz, 2H), 7.10 (d, J=8.4 Hz, 2H), 4.64 (s, 2H), 3.33 (t, J=7.4 Hz, 2H), 2.82-2.60 (m, 5H), 2.34 (t, J=7.6 Hz, 2H), 1.33 (q, J=7.4 Hz, 2H), 0.87 (t, J=7.2, 3H).

### Step 5: Preparation of 4-((1-propylazetidin-3-yl)methyl)benzaldehyde

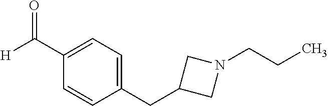 [SMILES: CCCN1CC(Cc2ccc(C=O)cc2)C1]

To a solution of(4-((1-propylazetidin-3-yl)methyl)phenyl)methanol (8.050 g, 36.7 mmol, 1.0 equiv.) in DCM was added activated manganese dioxide (31.909 g, 367.0 mmol, 10.0 equiv.). The reaction was stirred at RT for 3 days. An aliquot was taken and worked up. NMR analysis of the aliquot indicated that the reaction was complete. The mixture was filtered through a Celite® pad which was rinsed with DCM and EA. The filtrate was concentrated and resulting residue was loaded to a silica gel column (120 g, 0-10% MeOH/DCM) to afford the title compound as a light yellow oil (5.8 g, 72.7%) which solidified slowly at −20° C.

¹H NMR (300 MHz, CDCl₃) δ 9.98 (s, 1H), 7.81 (d, J=8.4 Hz, 2H), 7.32 (d, J=7.5 Hz, 2H), 3.66 (t, J=7.5 Hz, 2H), 3.22 (t, J=7.5 Hz, 2H), 3.03-2.94 (m, 3H), 2.61 (t, J=7.5 Hz, 2H), 1.50 (q, J=7.4 Hz, 2H), 0.93 (t, J=7.4 Hz, 3H).

### Preparation 5: Preparation of 3-fluoro-4-((1-propylazetidin-3-yl)oxy)benzaldehyde

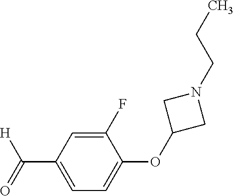 [SMILES: CCCN1CC(Oc2ccc(C=O)cc2F)C1]

A solution of 3-fluoro-4-iodobenzaldehyde (0.800 g, 3.2 mmol, 1.0 equiv.), 95.0% 1-propylazetidin-3-ol (1.261 g, 10.4 mmol, 3.3 equiv.) in butyronitrile (1 mL), 1,10-phenanthroline (0.058 g, 0.3 mmol, 0.1 equiv.), and cesium carbonate (2.294 g, 7.0 mmol, 2.2 equiv.) were added to a 48 mL glass pressure bottle. The mixture was degassed and blanketed with argon (3 times), then Cu(I) iodide (0.616 g, 3.2 mmol, 1.0 equiv.) was added. The mixture was degassed and blanketed with argon an additional 3 times. The reaction mixture was heated at 120° C. for 40 h. TLC (20% EA/Hex) indicated there was still starting material present. TLC (5% MeOH/DCM) indicated there was a new spot less polar than the starting aldehyde. The reaction was cooled to RT and diluted with EA and the mixture was sonicated.

The mixture was filtered through a Celite® pad. The resulting dark brown residue was purified on a silica gel column (12 g, 0-10% MeOH/DCM) to provide a dark oil that contained impure product. The material was dissolved in acetonitrile and further purified on preparative HPLC (10-90% acetonitrile/H₂O, 20 min) to provide the title compound as a light brown oil (0.073 g, 9.6%).

LCMS: [M+1]⁺, 238.5.

¹H NMR (CDCl₃, 300 MHz) δ 9.85 (s, 1H), 7.64-7.58 (m, 2H), 6.83 (t, J=7.9 Hz, 1H), 4.90 (t, J=5.8 Hz, 1H), 3.90-3.85 (m, 2H), 3.18-3.13 (m, 2H), 2.50 (t, J=7.5 Hz, 2H), 1.45-1.37 (m, 2H), 0.92 (t, J=7.6 Hz, 3H).

### Example 1: Preparation of 3-(4-hydroxyphenyl)-4-methyl-2-(4-(1-propylazetidin-3-yl)methyl)methyl-2H-chromen-7-ol (Compound 101)

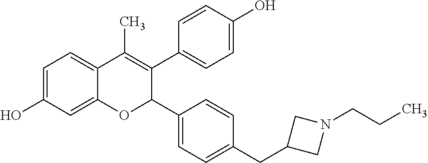 [SMILES: CCCN1CC(Cc2ccc(C3Oc4cc(O)ccc4C(C)=C3c3ccc(O)cc3)cc2)C1]

### Step 1: Preparation of 2-(4-((1-propylazetidin-3-ylmethyl)phenyl)-7-((tetrahydro-2H-pyran-2-yl)oxy)-3-(4-((tetrahydro-2H-pyran-2-yl)oxy)phenyl)chroman-4-one

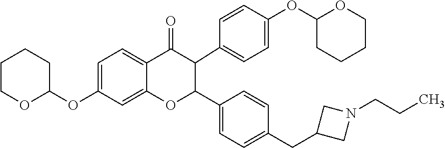 [SMILES: CCCN1CC(Cc2ccc(C3Oc4cc(OC5CCCCO5)ccc4C(=O)C3c3ccc(OC4CCCCO4)cc3)cc2)C1]

1-(2-Hydroxy-4-((tetrahydro-2H-pyran-2-yl)oxy)phenyl)-2-(4-((tetrahydro-2H-pyran-2-yl)oxy)phenyl)ethanone (1.594 g, 3.9 mmol, 1.1 equiv.) was added to a 100 mL three-neck flask. 2-Butanol (30 mL) and the product of Preparation 4, 4-((1-propylazetidin-3-yl)methyl)benzaldehyde (0.80 g, 3.7 mmol, 1.0 equiv.), were added to the flask to provide a suspension. Piperidine (0.36 mL, 3.6 mmol, 1.0 equiv.) and DBU (0.36 mL, 2.4 mmol, 0.7 equiv.) were added to the mixture to provide a white suspension. The flask was fitted with a Dean-Stark trap and condenser and heated in an oil bath at 130° C. The white suspension became a light yellow solution when the temperature reached 65° C. Half the solvent (15 mL) was collected over 90 min. The reaction was heated for a further 12 h. The reaction was cooled to RT and diluted with EA (30 mL) and washed with water and brine. The organic layer was dried over anhydrous sodium sulfate, filtered, and concentrated. The resulting residue was dissolved in DCM and loaded to a silica gel column and purified (24 g, 0-10% MeOH/DCM) to afford the title compound as an off-white solid (1.66 g, 73.7%).

LCMS: [M+1]⁺, 611.9.

### Step 2: Preparation of 4-methyl-2-(4-((1-propylazetidin-3-yl)methyl)phenyl)-7-((tetrahydro-2H-pyran-2-yl)oxy)-3-(4-((tetrahydro-2H-pyran-2-yl)oxy)phenyl)chroman-4-ol

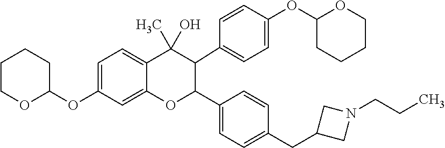 [SMILES: CCCN1CC(Cc2ccc(C3Oc4cc(OC5CCCCO5)ccc4C(C)(O)C3c3ccc(OC4CCCCO4)cc3)cc2)C1]

To a solution of 90.0% 2-(4-((1-propylazetidin-3-yl)methyl)phenyl)-7-((tetrahydro-2H-pyran-2-yl)oxy)-3-(4-((tetrahydro-2H-pyran-2-yl)oxy)phenyl)chroman-4-one (0.300 g, 0.4 mmol, 1.0 equiv.) in THF (100 mL) at 0° C. was added methylmagnesium chloride 3.0 M solution in THF (0.80 mL, 2.4 mmol, 5.4 equiv.) dropwise via a syringe. After complete addition of the Grignard reagent, the reaction was removed from the ice-bath and allowed to reach RT and stirred for 1 h. LCMS analysis indicated that the reaction was incomplete. An additional amount of methylmagnesium chloride (2 mL) was added at 0° C. and the reaction was stirred at RT for 5 h. LCMS indicated that the reaction was complete ([M+1]⁺, 628). The reaction was cooled in an ice-bath and quenched with sodium sulfate decahydrate. The suspension was filtered through a pad of Celite® rinsed with EA (3 times). The filtrate was dried over anhydrous sodium sulfate, filtered and concentrated to provide the title compound as a light yellow foam, which was used directly in the next step.

### Step 3: Preparation of 3-(4-hydroxyphenyl)-4-methyl-2-(4-((1-propylazetidin-3-yl)methyl)phenyl)-2H-chromen-7-ol

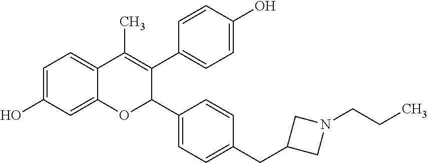 [SMILES: CCCN1CC(Cc2ccc(C3Oc4cc(O)ccc4C(C)=C3c3ccc(O)cc3)cc2)C1]

4-methyl-2-(4-((1-propylazetidin-3-yl)methyl)phenyl)-7-((tetrahydro-2H-pyran-2-yl)oxy)-3-(4-((tetrahydro-2H-pyran-2-yl)oxy)phenyl)chroman-4-ol was mixed with 80% acetic acid/H₂O (5 mL) and evacuated and blanketed with nitrogen. The mixture was heated at 90° C. for 30 min. HPLC indicated that the reaction was complete. The reaction was concentrated and released from vacuum under nitrogen. Saturated aq. sodium bicarbonate and EA was added to the residue. The formed suspension was stirred for 30 min at RT under nitrogen. The organic layer was separated and washed with sodium bicarbonate solution (1 time) and brine, dried over anhydrous sodium sulfate, filtered and concentrated to provide a pinkish light brown solid. The solid was purified on a silica gel column (24 g, 0-15% MeOH/DCM) to afford the title compound as a light yellow foam, which solidified on standing. The solid was dried in a vacuum oven at 60° C. for 24 h to afford the title compound (0.12 g, 59.7%).

LCMS: [M+1]⁺, 442.6. HPLC: 96.93%.

¹H NMR (300 MHz, DMSO-d₆) δ 9.44 (s, 1H), 7.18-7.00 (m, 7H), 6.71 (d, J=8.7 Hz, 2H), 6.32 (dd, J=5.4, 2.4 Hz, 1H), 6.08 (d, J=2.1 Hz, 1H), 5.93 (s, 1H), 3.18 (t, J=6.6 Hz, 2H), 2.70-2.66 (m, 5H), 2.01 (s, 3H), 1.22 (q, J=7.5 Hz, 2H), 0.79 (t, J=7.5 Hz, 3H).

### Examples 2 and 3: Separation of 3(4-hydroxyphenyl)4-methyl-2-(4((1-propylazetidin-3-yl)methyl)phenyl)-2H-chromen-7-ol Compound 102 (S-isomer) and Compound 103 (R-isomer)

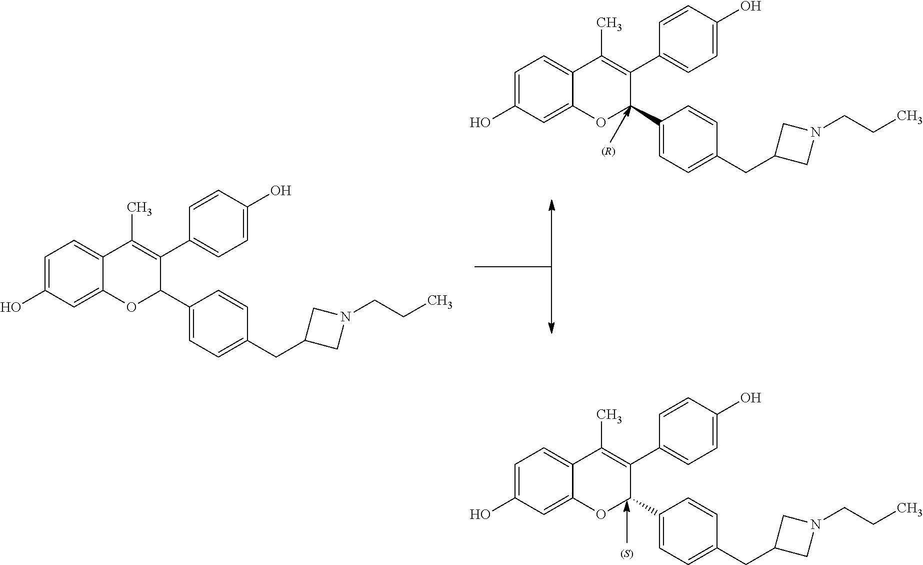

3-(4-Hydroxyphenyl)-4-methyl-2-(4-((1-propylazetidin-3-yl)methyl)phenyl)-2H-chromen-7-ol (0.060 g, 0.1 mmol) was dissolved into 2 mL of absolute ethanol. The solution was purified by preparative chromatography with 400 to 600 μL injections over 5 runs. Fractions of each peak were pooled and concentrated via rotovap separately to provide light yellow solids. The solids were dried under high vacuum at 50° C. for 2 days. Peak 1,

Compound 102: 16.7 mg; Peak 2, Compound 103: 15.4 mg.

Analytical HPLC

Column: ChiralPak AD-H, 250×4.6 mm

Temperature: 25° C.

Flow: 1 mLmin

Solvent system: 20% denatured EtOH (90% EtOH, 5% IPA, 5% MeOH) in Hex with 0.1% DEA

Chiral Retention Times (minutes)

Peak 1, Compound 102: 5.51

Peak 2, Compound 103: 6.57

Purification on Preparative HPLC

Column: ChiralPak AD-H, 250×20 mm

Temperature: not regulated

Flow: 15 mL/min

Solvent system: 20% denatured EtOH (90% EtOH, 5% IPA, 5% MeOH) in Hex with 0.1% DEA

Chiral Retention Times (minutes)

Peak 1, Compound 102: 6.8

Peak 2, Compound 103: 8.3

Analytical Data

Peak 1, Compound 102

HPLC: 98.84%

ee: 100%

LCMS: 442.7

¹H NMR (400 MHz, CD₃OD) δ 7.22 (d, J=7.2 Hz, 2H), 7.15 (d, J=8.4 Hz, 1H), 7.02 (dd, J=8.4 Hz, 4H), 6.73 (d, J=7.2 Hz, 2H), 6.38 (d, J=8.4 Hz, 1H), 6.15 (s, 1H), 5.84 (s, 1H), 3.53-3.51 (m, 2H), 3.07-3.05 (m, 2H), 2.79 (s, 2H), 2.52 (t, J=7.8 Hz, 2H), 2.05 (s, 3H), 1.38 (pent, J=7.6 Hz, 2H), 1.35-1.20 (m, 1H), 0.91 (t, J=7.2 Hz, 3H).

Peak 2, Compound 103

HPLC: 100%

ee: 100%

LCMS: 442.4

¹H NMR (400 MHz, CD₃OD) δ: same as above

### Example 4: Preparation of 3-(4-hydroxyphenyl)-4-methyl-2-(4-((1-propylazetidin-3-yl)oxy)phenyl)-2H-chromen-7-ol (Compound 104)

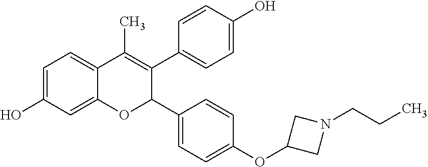 [SMILES: CCCN1CC(Oc2ccc(C3Oc4cc(O)ccc4C(C)=C3c3ccc(O)cc3)cc2)C1]

### Step 1: Preparation of 2-(4-iodophenyl)-7-((tetrahydro-2H-pyran-2-yl)oxy)-3-(4-((tetrahydro-2H-pyran-2-yl)oxy)phenyl)chroman-4-one

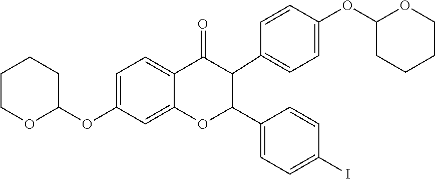 [SMILES: O=C1c2ccc(OC3CCCCO3)cc2OC(c2ccc(I)cc2)C1c1ccc(OC2CCCCO2)cc1]

1-(2-Hydroxy-4-((tetrahydro-2H-pyran-2-yl)oxy)phenyl)-2-(4-((tetrahydro-2H-pyran-2-yl)oxy)phenyl)ethanone (293.0 g, 0.71 mol, 1.0 equiv.) was added to a three-neck 5 L round bottom flask. 2-Butanol (1.25 L) and 97.0% 4-iodobenzaldehyde (169.9 g, 0.71 mol, 1.0 equiv.) were added to the flask to provide a suspension. Piperidine (23.5 mL, 0.24 mol, 0.3 equiv.) and DBU (36.4 mL, 0.24 mol, 0.3 equiv.) were added to the suspension. The flask was fitted with a Dean-Stark apparatus, a condenser, a thermometer with an inlet adapter, and a stir bar. The reaction was heated under a nitrogen atmosphere with a mantle to provide an orange solution at 78° C. Heating was continued to reflux. Half the solvent (610 mL) was collected over 1.5 h. The reaction was heated for another hour without collecting 2-butanol. The solution gradually darkened to a red color. The mantle was removed and the flask was allowed to cool to 90° C. 2-propanol (500 mL) was then added and the reaction mixture stirred. A large mass formed at the bottom of the flask when the temperature dropped below 50° C. The reaction was stirred for 2 days. The flask contained a solid cake and a clear solution. The supernatant was decanted and the solid was mixed with 2-propanol (100 mL) and diethyl ether (80 mL) and slowly warmed with agitation using a metal spatula until the mixture stirred freely with a stir bar. The solid mass became a viscous suspension. 25% diethyl ether/Hex (300 mL) was added to the mixture and the suspension stirred for 1 h. The suspension was filtered and the solid washed with 25% diethyl ether/Hex to provide the title compound as a white powder (240.0 g, 54%). The filtrate was concentrated and precipitation was performed, as above, to provide further compound (40.0 g, 9%). The filtrates and the supernatant were combined, concentrated, adsorbed onto silica gel and purified on silica gel with 30% EA/Hex to provide the title compound as a yellow foam (137.0 g, 31%, m.p. 136-138° C.). Overall yield 94%.

LCMS: [M+1]⁺, 626.3.

¹H NMR (300 MHz, CDCl₃) δ 7.93-7.89 (m, 1H), 7.57 (d, J=8.1 Hz, 2H), 6.95-6.70 (m, 8H), 5.69-5.33 (m, 3H), 3.65-3.55 (m, 2H), 4.00-3.77 (m, 3H), 2.04-1.96 (m, 2H), 1.88-1.80 (m, 4H), 1.69-1.64 (m, 6H).

### Step 2: Preparation of 2-(4-iodophenyl)-4-methyl-7-((tetrahydro-2H-pyran-2-yl)oxy)-3-(4-((tetrahydro-2H-pyran-2-yl)oxy)phenyl)chroman-4-ol

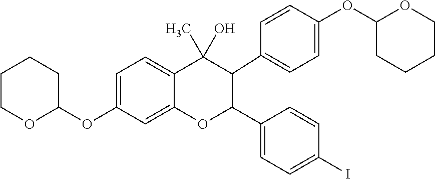 [SMILES: CC1(O)c2ccc(OC3CCCCO3)cc2OC(c2ccc(I)cc2)C1c1ccc(OC2CCCCO2)cc1]

To a solution of 2-(4-iodophenyl)-7-((tetrahydro-2H-pyran-2-yl)oxy)-3-(4-((tetrahydro-2H-pyran-2-yl)oxy)phenyl)chroman-4-one (226.0 g, 325 mmol, 1.0 equiv.) in THF (300 mL) at 0° C. was added methylmagnesium chloride (3.0 M solution) in THF (220 mL, 660.0 mmol, 2.0 equiv.) dropwise via a pressure equalizing addition funnel over a period of 1 h. After complete addition of the Grignard reagent, the reaction was removed from the ice-bath and allowed to reach RT and stirred overnight. TLC (20% EA/Hex) analysis indicated that the reaction was complete. The reaction was cooled in an ice-bath and quenched with saturated ammonium chloride (250 mL) to provide a thick yellow suspension. The suspension was filtered through a pad of Celite® and the filter cake was washed with saturated ammonium chloride (3×100 mL), followed by EA (3×100 mL). The filtrate was poured into a separatory funnel and the layers separated. The aqueous layer was extracted with EA (3×100 mL). The combined organic layers were dried over anhydrous sodium sulfate, filtered and concentrated. The resulting residue was purified through silica gel (10-30% EA/Hex) to afford the title compound as an off-white foam (160.0 g, 76.7%).

LCMS: [M−18+1]⁺, 624.2.

### Step 3: Preparation of 3-(4-hydroxyphenyl)-4-methyl-2-(4-((1-propylazetidin-3-yl)oxy)phenyl-2H-chromen-7-ol

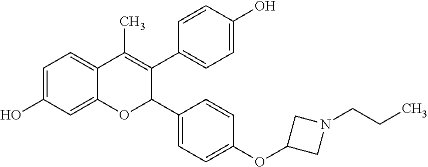 [SMILES: CCCN1CC(Oc2ccc(C3Oc4cc(O)ccc4C(C)=C3c3ccc(O)cc3)cc2)C1]

To a 250 mL flask were added, 2-(4-iodophenyl)-4-methyl-7-((tetrahydro-2H-pyran-2-yl)oxy)-3-(4-((tetrahydro-2H-pyran-2-yl)oxy)phenyl)chroman-4-ol (17.65 g, 27.5 mmol, 1.0 equiv.), 95.0% 1-propylazetidin-3-ol, which was the product of Preparation 2 (9.99 g, 82.4 mmol, 3.0 equiv.), butyronitrile (70 mL), 1,10-phenanthroline (0.990 g, 5.5 mmol, 0.2 equiv.), and cesium carbonate (17.94 g, 55.1 mmol, 2.0 equiv.). The mixture was degassed and blanketed with argon (3 times). Copper(I) iodide (5.23 g, 27.5 mmol, 1.0 equiv.) was added to the degassed mixture. The mixture was further degassed and blanketed with argon an additional 3 times. The reaction mixture was heated at 125° C. for 40 h. The reaction was cooled to ambient temperature and diluted with EA. The mixture was filtered through a pad of Celite®. The filtrate was washed with saturated aq. ammonium chloride (50 mL), water (50 mL), brine, dried over sodium sulfate, filtered and concentrated in vacuo. The dark residue was cooled to ambient temperature and evacuated and blanketed with nitrogen. 80% acetic acid/water (50 mL) was added to the residue and the reaction mixture was evacuated and blanketed with nitrogen (2 times). The suspension was heated at 90° C. for 1 h. HPLC and LCMS indicated that the reaction was complete and the desired mass was observed. The reaction was concentrated and cooled to RT and stirred with saturated aq. sodium bicarbonate and EA for 30 min. After separation, the aqueous layer was extracted with EA (100 mL). The combined organic layers were washed with water (100 mL), brine (100 mL), dried over anhydrous sodium sulfate, filtered and concentrated. The resulting residue was purified on a silica gel column (200 g, 0-15% MeOH/DCM gradient) to provide the title compound (6 g). This material was further purified on a silica gel column (200 g, 0-15% MeOH/DCM, held at 6% MeOH/DCM). The middle fractions were collected to afford the title compound (2 g). The fractions containing impurities were collected and concentrated to provide a brown foam. The brown foam was suspended in MeOH and sonicated. The resulting solid was filtered and rinsed with MeOH. All batches were combined and analyzed by HPLC. The solid was dried in a vacuum oven at 60° C. overnight to afford the title compound as an off-white powder (3.64 g, 29%).

LCMS: [M+1]⁺, 444.6. HPLC: 98.08%.

¹H NMR (300 MHz, DMSO-d₆) δ 9.45 (s, 1H), 9.44 (s, 1H), 7.18 (d, J=8.1 Hz, 2H), 7.12-7.05 (m, 3H), 6.72-6.67 (m, 4H), 6.32 (dd, J=5.4, 2.4 Hz, 1H), 6.07 (d, J=2.4 Hz, 1H), 5.90 (s, 1H), 4.67 (t, J=5.6 Hz, 1H), 3.67 (t, J=6.8 Hz, 2H), 2.85 (t, J=6.0 Hz, 2H), 2.34 (t, J=7.1 Hz, 2H), 2.01 (s, 3H), 1.26 (q, J=7.3 Hz, 2H), 0.81 (d, J=7.3 Hz, 3H).

### Examples 5 and 6: Separation of 3(4-hydroxyphenyl)-4-methyl-2-(44(1-propylazetidin-3-yl)oxy)phenyl)-2H-chromen-7-ol Compound 105 (S-isomer) and Compound 106 (R-isomer)

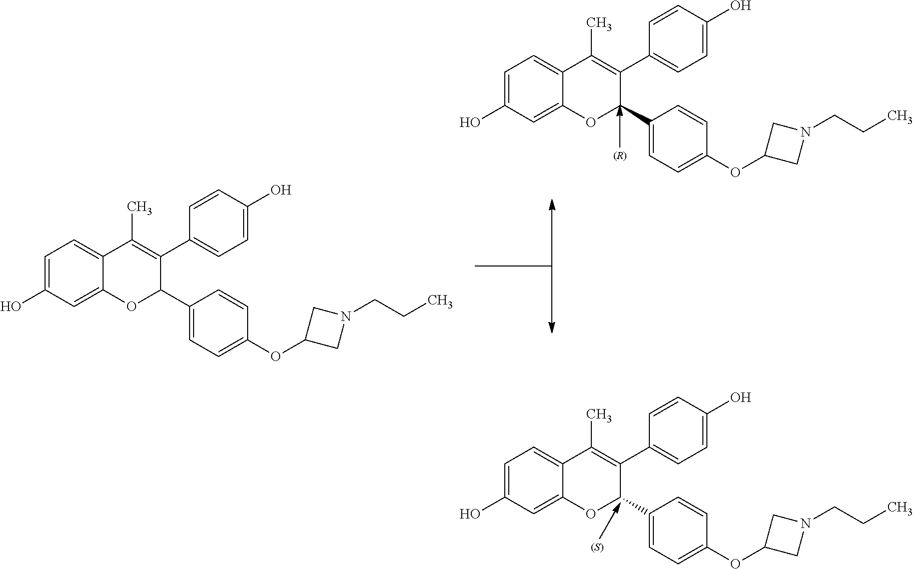

3-(4-Hydroxyphenyl)-4-methyl-2-(4-((1-propylazetidin-3-yl)oxy)phenyl)-2H-chromen-7-ol (0.200 g, 0.5 mmol, 1.0 equiv.) was dissolved into 18 mL of absolute ethanol. The solution was purified by preparative chromatography with 1500 μL injections over 12 runs with 10 mL fractions. Fractions of each peak were pooled and concentrated via rotovap separately to provide light yellow solids. The solids were dried under high vacuum at 50° C. for 2 days. Peak 1, Compound 105: 81 mg; Peak 2, Compound 106: 72 mg.

Analytical HPLC

Column: ChiralPak AD-H, 250×4.6 mm

Temperature: 25° C.

Flow: 1 mL/min

Solvent system: 20% denatured EtOH (90% EtOH, 5% IPA, 5% MeOH) in Hex with 0.1% DEA

Chiral Retention Times (minutes)

Peak 1, Compound 105: 14.03

Peak 2, Compound 106: 19.26

Purification on Preparative HPLC

Column: ChiralPak AD-H, 250×20 mm

Temperature: not regulated

Flow: 15 mL/min

Solvent system: 20% denatured EtOH (90% EtOH, 5% IPA, 5% MeOH) in Hex with 0.1% DEA

Chiral Retention Times (minutes)

Peak 1, Compound 105: 15.0

Peak 2, Compound 106: 19.8

Analytical Data

Peak 1, Compound 105

HPLC: 96.7

ee: 96.6%

LCMS: (M+1)⁺, 444.4

¹H NMR (300 MHz, CD₃OD) δ 7.22 (d, J=8.1 Hz, 2H), 7.13 (d, J=8.7 Hz, 1H), 7.02 (d, J=8.7 Hz, 2H), 6.72 (d, J=8.7 Hz, 2H), 6.65 (d, J=8.7 Hz, 1H), 6.36 (dd, J=2.1, 8.1 Hz, 1H), 6.11 (d, J=2.4 Hz, 1H), 5.78 (s, 1H), 4.80-4.70 (m, 1H), 3.84-3.80 (m, 2H), 3.31-3.26 (m, 2H), 2.59 (t, J=4.5 Hz, 2H), 2.02 (s, 3H), 1.44-1.32 (m, 2H), 0.90 (t, J=7.2 Hz, 3H).

Peak 2, Compound 106

HPLC: 100%

ee: 96.9%

LCMS: (M+1)⁺, 444.4

¹H NMR (300 MHz, CD₃OD) δ: same as above

### Example 7: Preparation of 2-(3-fluoro-4-((1-propylazetidin-3-yl)oxy)phenyl-3-(4-hydroxyphenyl)-4-methyl-2H-chromen-7-(Compound 107)

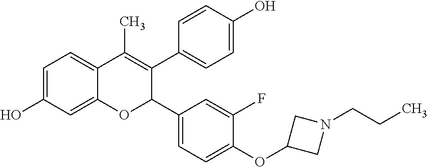 [SMILES: CCCN1CC(Oc2ccc(C3Oc4cc(O)ccc4C(C)=C3c3ccc(O)cc3)cc2F)C1]

### Step 1: Preparation of 2-(3-fluoro-4-((1-propylazetidin-3-yl)oxy)phenyl)-7-((tetrahydro-2H-pyran-2-yl)oxy)-3-(4-((tetrahydr-2H-pyran-2-yl oxy)phenyl)chroman-4-one

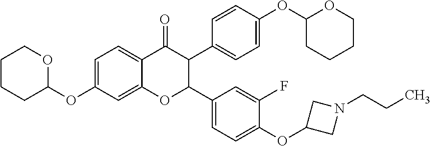 [SMILES: CCCN1CC(Oc2ccc(C3Oc4cc(OC5CCCCO5)ccc4C(=O)C3c3ccc(OC4CCCCO4)cc3)cc2F)C1]

1-(2-hydroxy-4-((tetrahydro-2H-pyran-2-yl)oxy)phenyl)-2-(4-((tetrahydro-2H-pyran-2-yl)oxy)phenyl)ethanone (0.497 g, 1.2 mmol, 1.1 equiv.) was added to a 100 mL three-neck flask. 2-Butanol (1 mL) and the product of Preparation 5, 3-fluoro-4-((1-propylazetidin-3-yl)oxy)benzaldehyde (0.260 g, 1.1 mmol, 1.0 equiv.) were added to the flask to give a suspension. Piperidine (0.036 mL, 0.4 mmol, 0.3 equiv.) and 1,8-diazabicyclo[5.4.0]undec-7-ene (0.054 mL, 0.4 mmol, 0.3 equiv.) were added to the mixture. The flask was fitted with a Dean-Stark apparatus and condenser and heated in an oil bath at 130° C. Half the solvent (5.5 mL) was collected over 20 min. The reaction was heated for an additional 12 h at 120° C. LCMS indicated the desired product and a minor mono-deprotected product. The reaction was cooled to RT and concentrated. The resulting residue was dissolved in DCM and loaded onto a silica gel column and purified (12 g, 50% EA/Hex, then 5% MeOH/DCM). The fractions that had a desired mass ([M+1]⁺, 632.1) were then concentrated to give a pale yellow foam (0.538 g, 77.7%) containing the desired product containing small amounts of impurities.

### Step 2. Preparation of 2-(3-fluoro-4-((1-propylazetidin-3-yl)ox)phenyl)-3-(4-hydroxyphenyl)-4-methyl-2H-chromen-7-ol

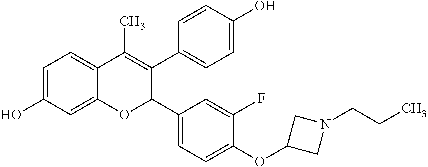 [SMILES: CCCN1CC(Oc2ccc(C3Oc4cc(O)ccc4C(C)=C3c3ccc(O)cc3)cc2F)C1]

To a solution of 90.0% 2-(3-fluoro-4-((1-propylazetidin-3-yl)oxy)phenyl)-7-((tetrahydro-2H-pyran-2-yl)oxy)-3-(4-((tetrahydro-2H-pyran-2-yl)oxy)phenyl)chroman-4-one (0.537 g, 0.8 mmol, 1.0 equiv.) in THF (7 mL) at 0° C., was added methylmagnesium chloride 3.0 M solution in THF (1.3 mL, 3.8 mmol, 5.0 equiv.) dropwise. After complete addition of the Grignard reagent, the reaction was removed from ice-bath, allowed to reach RT and stirred for 12 h. LCMS indicated the mass of desired product along with starting material. TLC (5% MeOH/DCM) did not differentiate product from starting material. One equivalent of Grignard reagent was added and the reaction mixture was stirred at RT for 1 h. LCMS indicated the mass of the product. The reaction was cooled in an ice-bath and quenched with saturated aq. ammonium chloride. The ice-bath was removed and the resulting suspension was stirred at RT for 15 min. The suspension was diluted with EA and separated. The organic layer was washed with water, brine, dried over anhydrous sodium sulfate, filtered and concentrated to give a light yellow foam (473 mg). 80% acetic acid/H₂O (12.5 mL) was added to the residue and the reaction was evacuated and blanketed with nitrogen. The mixture was heated at 90° C. for 1 h. The reaction was concentrated, and saturated NaHCO₃ solution and EA were added thereto. The suspension was stirred for 40 min at RT. The layers were separated, and the organic layer was washed with saturated sodium bicarbonate solution (1 time), water, brine, dried over anhydrous Na₂SO₄, filtered and concentrated to give a greenish residue. The residue was purified on a silica gel column (40 g, 0-15 MeOH/DCM) to afford the title compound along with impurities. TLC (7% MeOH/DCM) indicated all fractions have the same R_{f} value but LCMS indicated two peaks. The fractions containing product were concentrated to an oil and triturated with MeOH. The solid was centrifuged and the first crop (87 mg, HPLC 95.2%) was collected. The mother liquor was concentrated and the process repeated. The second crop (59 mg, HPLC 99.4%) was collected.

LCMS: [M+1]⁺, 462.2.

¹H NMR (300 MHz, DMSO-d₆) δ 9.47 (s, 1H), 9.46 (s, 1H), 7.13-6.99 (m, 5H), 6.84-6.71 (m, 3H), 6.34 (dd, J=8.7, 2.4 Hz, 1H), 6.11 (d, J=2.4 Hz, 1H), 5.93 (s, 1H), 4.75-4.71 (m, 1H), 3.68-3.64 (m, 2H), 2.91-2.86 (m, 2H), 2.35 (t, J=6.9 Hz, 2H), 2.02 (s, 3H), 1.30-1.23 (m, 2H), 0.82 (t, J=7.2 Hz, 3H).

### Examples 8 and 9: Separation of 2-(3-fluoro-4-((1-propylazetidin-3-yl)oxy)phenyl)-3-(4-hydroxyphenyl)-4-methyl-2H-chromen-7-ol, Compound 108 (S-Isomer) and Compound 109 (R-isomer)

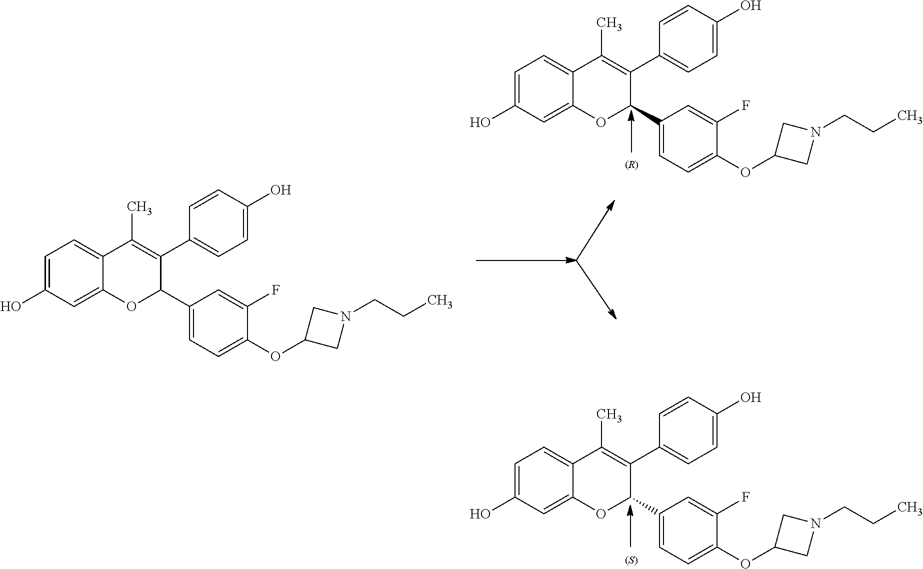

2-(3-fluoro-4-((1-propylazetidin-3-yl)oxy)phenyl)-3-(4-hydroxyphenyl)-4-methyl-2H-chromen-7-ol (0.023 g) was suspended into 4 mL of MeOH and dissolved with gentle heating. The solution was purified by preparative chromatography with four 1000 μL injections with 5 mL fractions or 10 mL fractions. Fractions of each peak were pooled and concentrated separately using a rotary evaporator to provide light yellow solids. The solids were dried under high vacuum at 50° C. for 1 day. Peak 1, Compound 108: 8.1 mg; Peak 2, Compound 109: 7.2 mg.

Analytical HPLC

Column: ChiralPak AD-H, 250×4.6 mm

Temperature: 25° C.

Flow: 1 mL/min

Solvent system: 20% [25% MeOH in denatured EtOH (5% MeOH, 5% IPA, 90% EtOH)] in Hex

Chiral Retention Times (minutes)

Peak 1, Compound 108: 10.5

Peak 2, Compound 109: 13.2

Purification on Preparative HPLC

Column: ChiralPak AD-H, 250×20 mm

Temperature: not regulated

Flow: 15 mLmin

Solvent system: 15% [25% MeOH in denatured EtOH (5% MeOH, 5% IPA, 90% EtOH)] in Hex

Chiral Retention Times (minutes)

Peak 1, Compound 108: 18.6

Peak 2, Compound 109: 23.7

Analytical Data

Peak 1, Compound 108

HPLC: 100%

ee: 100%

LCMS: (M+1)⁺, 462

¹H NMR (300 MHz, CD₃OD) δ 7.14 (d, J=9 Hz, 1H), 7.03-6.97 (m, 3H), 6.74-6.64 (m, 3H), 6.38 (dd, J=8.1, 2.4 Hz, 1H), 6.15 (d, J=2.1 Hz, 1H), 5.79 (s, 1H), 4.77-4.71 (m, 1H), 3.74-3.65 (m, 2H), 3.18-3.11 (m, 2H), 2.46 (t, J=7.8 Hz, 2H), 2.03 (s, 3H), 1.41-1.32 (m, 2H), 0.89 (t, J=7.5 Hz, 3H).

Peak 2, Compound 109

HPLC: 89.8%

ee: 94.9%

LCMS: (M+1)⁺462

¹H NMR (300 MHz, CD₃OD) δ: same as above.

### Example 10: Preparation of 3(4-hydroxyphenyl)-4-methyl-2-(4-((1-(3,3,3-trifluoropropyl)azetidin-3-yl)oxy)phenyl)-2H-chromen-7-ol

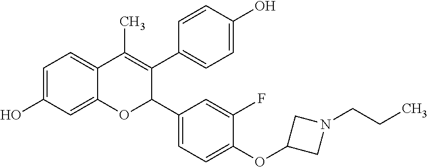 [SMILES: CCCN1CC(Oc2ccc(C3Oc4cc(O)ccc4C(C)=C3c3ccc(O)cc3)cc2F)C1]

Ullmann coupling was carried out in a manner substantially similar to the procedure described in Example 4, Step 3, using the product of Preparation 3 to afford the above-identified compound.

¹H NMR (300 MHz, CD₃OD) δ 7.20 (d, J=8.7 Hz, 2H), 7.12 (d, J=8.1 Hz, 1H), 6.99 (d, J=8.7 Hz, 2H), 6.71 (d, J=8.4 Hz, 2H), 6.63 (d, J=8.7 Hz, 2H), 6.35 (dd, J=5.6, 2.4 Hz, 1H), 6.12 (d, J=2.4 Hz, 1H), 5.78 (s, 1H), 4.75-4.72 (m, 1H), 3.78-3.73 (m, 2H), 3.19-3.14 (m, 2H), 2.74 (t, J=8.0 Hz, 2H), 2.28-2.20 (m, 2H), 2.03 (s, 3H).

### Examples 11 and 12: Separation of 3-(4-hydroxyphenyl)-4-methyl-2-(4-((1-(3,3,3-trifluoropropyl)azetidin-3-yl)oxy)phenyl)-2H-chromen-7-ol (Compound 110 (S-isomer and Compound 111 (R-isomer)

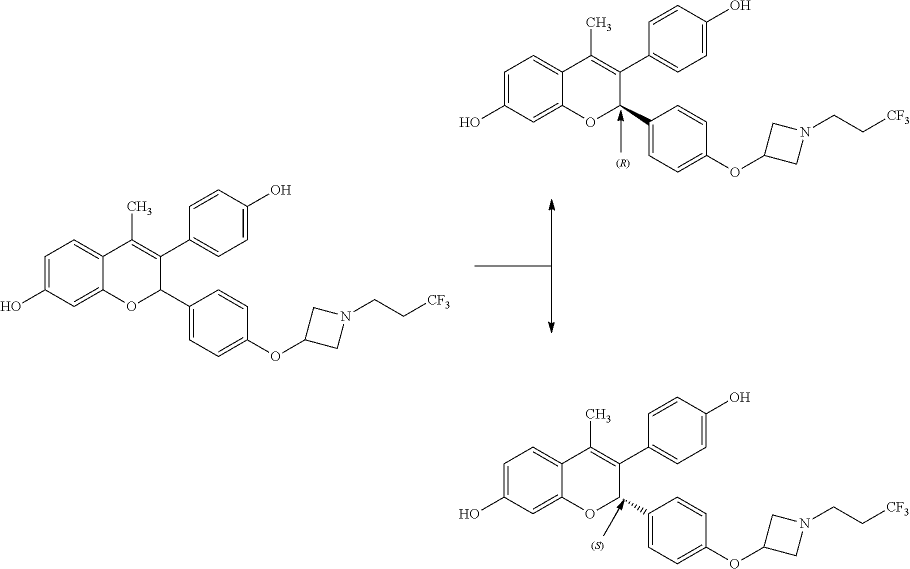

The separation was carried out in a manner substantially similar to the procedure described in Examples 2 and 3.

Analytical HPLC

Column: ChiralPak AD-H, 250×4.6 mm

Temperature: 25° C.

Flow: 1 mL/min

Solvent system: 15% IPA in Hex with 0.1% DEA

Chiral Retention Times (minutes)

Peak 1, Compound 111: 17.1

Peak 2, Compound 110: 18.4

Purification on Preparative HPLC

Column: ChiralPak AD-H, 250×20 mm

Temperature: not regulated

Flow: 15 mL/min

Solvent system: 15% IPA in Hex with 0.1% DEA

Chiral Retention Times (minutes)

Peak 1, Compound 111: 34.8

Peak 2, Compound 110: 39.8

Analytical Data

Peak 1, Compound 111

HPLC: 100%

ee: 97.9%

LCMS: (M+1)⁺, 498

¹H NMR (300 MHz, CD₃OD) δ 7.21 (d, J=8.7 Hz, 2H), 7.12 (d, J=8.1 Hz, 1H), 7.00 (d, J=8.1 Hz, 2H), 6.72 (d, J=8.7 Hz, 2H), 6.65 (d, J=8.7 Hz, 2H), 6.38 (dd, J=2.1, 8.7 Hz, 1H), 6.11 (d, J=2.4 Hz, 1H), 5.79 (s, 1H), 4.76-4.72 (m, 1H), 4.58 (s, 1H), 3.80-3.74 (m, 2H), 3.20-3.14 (m, 2H), 2.76 (t, J=7.5 Hz, 2H), 2.28-2.20 (m, 2H), 2.03 (s, 3H).

Peak 2, Compound 110

HPLC: 98.8%

ee: 87.8%

LCMS: (M+1)⁺, 498

¹H NMR (300 MHz, CD₃OD) δ: same as above.

### Example 13: Preparation of 2-(3-fluoro-4-((1-propylazetidin-3-yl)methyl)phenyl)-3-(4-hydroxyphenyl)-4-methyl-2H-chromen-7-ol

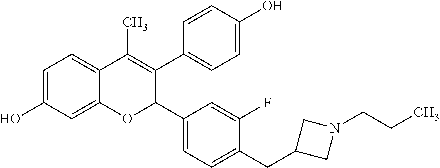 [SMILES: CCCN1CC(Cc2ccc(C3Oc4cc(O)ccc4C(C)=C3c3ccc(O)cc3)cc2F)C1]

### Step 1: Preparation of (2-fluoro-4-(methoxycarbonyl)benzyl)-triphenylphosphonium bromide

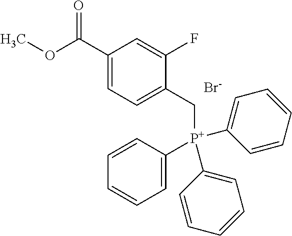 [SMILES: COC(=O)c1ccc(C[P+](c2ccccc2)(c2ccccc2)c2ccccc2)c(F)c1 ; [Br-]]

A solution of 95.0% methyl 4-(bromomethyl)-3-fluorobenzoate (2.40 g, 9.2 mmol, 1.0 equiv.) and triphenylphosphine (2.66 g, 10.2 mmol, 1.1 equiv.) in toluene (300 mL) was refluxed for 3 h and then cooled to RT. The precipitate was filtered and dried in vacuo at 50° C. to afford the title compound as a white powder (4.5 g, 95.7%).

¹H NMR (300 MHz, CDCl₃) δ 7.83-7.65 (m, 17H), 7.47 (d, J=10.8 Hz, 1H), 5.68 (d, J=12.5 Hz, 2H), 3.88 (s, 3H).

### Step 2: Preparation of methyl 3-fluoro-4-((1-propionylazetidin-3-ylidene)methyl)benzoate

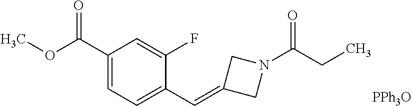 [SMILES: CCC(=O)N1CC(=Cc2ccc(C(=O)OC)cc2F)C1 ; "PPh3O"]

To a solution of (2-fluoro-4-(methoxycarbonyl)benzyl)triphenylphosphonium bromide (4.480 g, 8.8 mmol, 1.0 equiv.) in anhydrous DMSO (80 mL), was added potassium tert-butoxide (1.079 g, 9.6 mmol, 1.1 equiv.). The mixture was stirred at RT for 10 min to provide an orange suspension before 1-propionylazetidin-3-one (1.20 g, 9.4 mmol, 1.1 equiv.) in anhydrous DMSO (15 mL) was added to provide an orange solution. The reaction mixture was heated at 60° C. overnight. The mixture was cooled to RT and poured onto ice water (125 mL) and extracted with EA (4×80 mL). The combined organic extracts were washed with brine, dried over anhydrous sodium sulfate, filtered and concentrated. The resulting residue, containing the desired material and the by-product Ph₃PO, was used in the next step directly without purification.

LCMS: [M+1]⁺, 278.3.

¹H NMR (300 MHz, CDCl₃) δ 7.81-7.43 (m, 21H), 7.16-7.13 (m, 1H), 6.57 (s, 1H), 4.97-4.73 (m, 4H), 3.92 (s, 3H), 2.23-2.19 (m, 2H), 1.17 (t, J=7.5 Hz, 3H).

### Step 3: Preparation of methyl 3-fluoro-4-((1-propionylazetidin-3-yl)methyl)benzoate

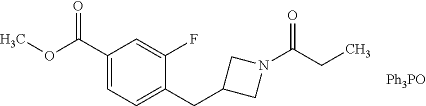

10% Pd/C (0.800 g, 0.3 mmol, 0.05 equiv.) was added to a solution of methyl 3-fluoro-4-((1-propionylazetidin-3-ylidene)methyl)benzoate (4.000 g, 14.4 mmol, 1.0 equiv.) (contained 1 equivalent of Ph₃PO from the previous reaction) in MeOH (100 mL). The mixture was evacuated and blanketed with nitrogen (2 times). The reaction was evacuated and flushed with a hydrogen balloon. The reaction was stirred at RT overnight. TLC (50% EA/Hex) indicated that the reaction was complete. LCMS indicated that the desired product formed. The mixture was evacuated and blanketed with nitrogen. Then it was filtered through a Celite® pad and rinsed with MeOH. The filtrate was concentrated and the resulting residue, containing the desired material and the by-product Ph₃PO, was used directly without additional purification.

LCMS: [M+1]⁺, 280.3.

### Step 4: Preparation of (3-fluoro-4-((1-propylazetidin-3-yl)methyl)phenyl)-methanol

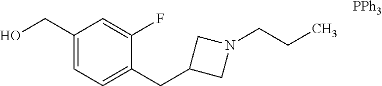 [SMILES: CCCN1CC(Cc2ccc(CO)cc2F)C1 ; "PPh3"]

To a solution of methyl 3-fluoro-4-((1-propionylazetidin-3-yl)methyl)benzoate (4.030 g, 14.4 mmol, 1.0 equiv.) in THF was added lithium aluminum hydride (2.132 g, 57.7 mmol, 4.0 equiv.) portionwise at 0° C. The mixture was stirred at 0° C. for 10 min, then the mixture was evacuated and blanketed with nitrogen. The greenish suspension was heated at 66° C. for 24 h. An aliquot was taken and worked up. NMR analysis of the aliquot indicated that the reaction was complete. The reaction mixture was cooled to 0° C. and quenched with sodium sulfate decahydrate. The mixture was allowed to warm to RT and stirred for 10 min before being filtered through a Celite® pad. The solid was washed with EA and MeOH. The filtrate was concentrated to a light yellow solid (3 g). The residue was dried under vacuum overnight (free of oxygen and solvent to prevent re-oxidation from triphenylphosphine to triphenylphosphine oxide). The residue was dissolved in DCM and loaded onto silica gel ˜6 g and chromatographed through silica gel (40 g, 0-10% MeOH/DCM, triphenylphosphine eluted immediately, followed by a small amount of starting material; the product was collected by monitoring UV 220 nm) to afford the title compound as a pale yellow oil (1.06 g, 42% yield from starting bromide).

LCMS: [M+1]⁺, 238.5.

¹H NMR (300 MHz, CDCl₃) δ 7.12-7.03 (m, 3H), 2.96-2.79 (m, 5H), 2.46-2.41 (m, 4H), 1.44-1.36 (m, 2H), 0.89 (t, J=7.5 Hz, 3H).

### Step 5: Preparation of 3-fluoro-4-((1-propylazetidin-3-yl)methyl)benzaldehyde

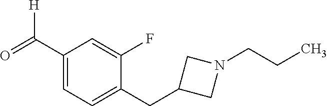 [SMILES: CCCN1CC(Cc2ccc(C=O)cc2F)C1]

Manganese dioxide (4.202 g, 48.3 mmol, 10.8 equiv.) was added to a solution of(3-fluoro-4-((1-propylazetidin-3-yl)methyl)phenyl)methanol (1.060 g, 4.5 mmol, 1.0 equiv.) in DCM. The reaction was stirred at RT for 3 days. An aliquot was taken and worked up. NMR analysis of the aliquot indicated that the reaction was complete. The mixture was filtered through a Celite® pad which was washed with DCM and EA. The filtrate was concentrated to afford the title compound as a yellow oil (0.95 g, 90.4%) and was used directly without purification in the next step.

LCMS: [M+1], 236.6.

¹H NMR (300 MHz, CDCl₃) δ 9.94 (s, 1H), 7.60-7.31 (m, 3H), 3.48-3.41 (m, 2H), 2.98-2.75 (m, 5H), 2.55-2.30 (m, 2H), 1.42-1.30 (m, 2H), 0.89 (t, J=7.5 Hz, 3H).

### Step 6: Preparation of 2-(3-fluoro-4-((1-propylazetidin-3-yl)methyl)phenyl)-7-((tetrahydro-2H-pyran-2-yloxy)-3-(4-((tetrahydro-2H-pyran-2-yloxy)phenyl)chroman-4-one

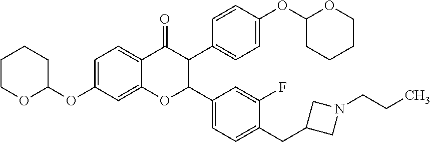 [SMILES: CCCN1CC(Cc2ccc(C3Oc4cc(OC5CCCCO5)ccc4C(=O)C3c3ccc(OC4CCCCO4)cc3)cc2F)C1]

1-(2-Hydroxy-4-((tetrahydro-2H-pyran-2-yl)oxy)phenyl)-2-(4-((tetrahydro-2H-pyran-2-yl)oxy)phenyl)ethanone (0.916 g, 2.2 mmol, 1.1 equiv.) was added to a 100 mL two-neck flask. 2-Butanol (20 mL) and the product of step 5, 3-fluoro-4-((1-propylazetidin-3-yl)methyl)benzaldehyde (0.475 g, 2.0 mmol, 1.0 equiv.), were added to the flask to provide a suspension. Piperidine (0.071 mL, 0.7 mmol, 0.4 equiv.) and 1,8-diazabicyclo[5.4.0]undec-7-ene (0.106 mL, 0.7 mmol, 0.4 equiv.) were added to the mixture to provide a white suspension. The flask was fitted with a Dean-Stark trap and condenser and heated in an oil bath at 130° C. The white suspension became a light yellow solution. Half the solvent (10 mL) was collected over 20 minutes. The reaction was heated for 12 h. TLC (5% MeOH/DCM) analysis indicated the aldehyde was consumed and there were two major spots. (The product was the major component, it was less polar than the aldehyde and the other spot was more polar than the aldehyde.) The reaction was cooled to RT and concentrated in vacuo at 30° C. The resulting residue was dissolved in DCM and loaded onto a silica gel column and purified (40 g, 0-10% MeOH/DCM) to afford the title compound as a pale yellow foam (0.468 g, 36.8%).

LCMS showed two peaks with the same mass [M+1]⁺, 630.1.

### Step 7: Preparation of 2-(3-fluor-4-((1-propylazetidin-3-yl)methyl)phenyl-4-methyl-7-((tetrahydro-2H-pyran-2-yl)oxy)-3-((tetrahydro-2H-pyran-2-yl)oxy)phenyl)-chroman-4-ol

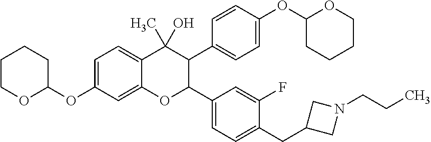 [SMILES: CCCN1CC(Cc2ccc(C3Oc4cc(OC5CCCCO5)ccc4C(C)(O)C3c3ccc(OC4CCCCO4)cc3)cc2F)C1]

To a solution of 90.0% 2-(3-fluoro-4-((1-propylazetidin-3-yl)methyl)phenyl)-7-((tetrahydro-2H-pyran-2-yl)oxy)-3-(4-((tetrahydro-2H-pyran-2-yl)oxy)phenyl)chroman-4-one (0.457 g, 0.7 mmol, 1.0 equiv.) in THF (50 mL) at 0° C. was added methylmagnesium chloride 3.0 M solution in THF (1 mL, 3.0 mmol, 4.6 equiv.) dropwise. After complete addition of the Grignard reagent, the reaction was removed from the ice-bath and allowed to reach RT and stirred for 1 h. LCMS indicated that the reaction was incomplete. Additional Grignard reagent (4 mL) was added to the reaction. LCMS indicated the reaction was almost complete. The reaction was cooled in an ice-bath and quenched with saturated aq. ammonium chloride. The suspension was diluted with EA and the layers separated. The organic layer was washed with water, brine, dried over anhydrous sodium sulfate, filtered and concentrated to provide an off-white foam (421 mg).

### Step 8: Preparation of 2-(3-fluoro-4-((1-propylazetidin-3-yl)methyl)phenyl)-3-(4-hydroxyphenyl)-4-methyl-2H-chromen-7-ol

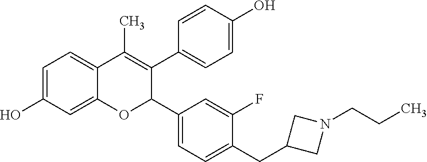 [SMILES: CCCN1CC(Cc2ccc(C3Oc4cc(O)ccc4C(C)=C3c3ccc(O)cc3)cc2F)C1]

2-(3-Fluoro-4-((1-propylazetidin-3-yl)methyl)phenyl)-4-methyl-7-((tetrahydro-2H-pyran-2-yl)oxy)-3-(4-((tetrahydro-2H-pyran-2-yl)oxy)phenyl)chroman-4-ol was mixed with 80% acetic acid/H₂O (10 mL) and evacuated and blanketed with nitrogen. The mixture was heated at 90° C. overnight. HPLC analysis indicated that the reaction was complete. The reaction was concentrated and saturated aq. NaHCO₃ and EA were added to the residue. The suspension was stirred for 40 min at RT. The organic layer was separated and washed with NaHCO₃ solution (1 time), water, brine, dried over anhydrous Na₂SO₄, filtered and concentrated to provide a brown solid. The solid was purified on a silica gel column (12 g, 0-15% MeOH/DCM) to afford the title compound as a light brown foam (0.16 g, 53.3%).

¹H NMR (300 MHz, CDCl₃) δ 7.15-6.91 (m, 6H), 6.73 (dd, J=8.1, 2.4 Hz, 2H), 6.38 (dd, J=5.4, 2.4 Hz, 1H), 6.17 (d, J=2.4 Hz, 1H), 5.84 (s, 1H), 3.40 (t, J=8.1 Hz, 2H), 2.90 (t, J=7.8 Hz, 2H), 2.79-2.71 (m, 4H), 2.43-2.39 (m, 2H), 2.03 (s, 3H), 1.38-1.32 (m, 2H), 0.89 (t, J=7.5 Hz, 3H).

### Examples 14 and 15: Separation of 2-(3-fluoro-4-((1-propylazetidin-3-yl)methyl)phenyl-3-(4-hydroxyphenyl)-4-methyl-2H-chromen-7-ol Compound 112 (S-Isomer) and Compound 113 (R-isomer)

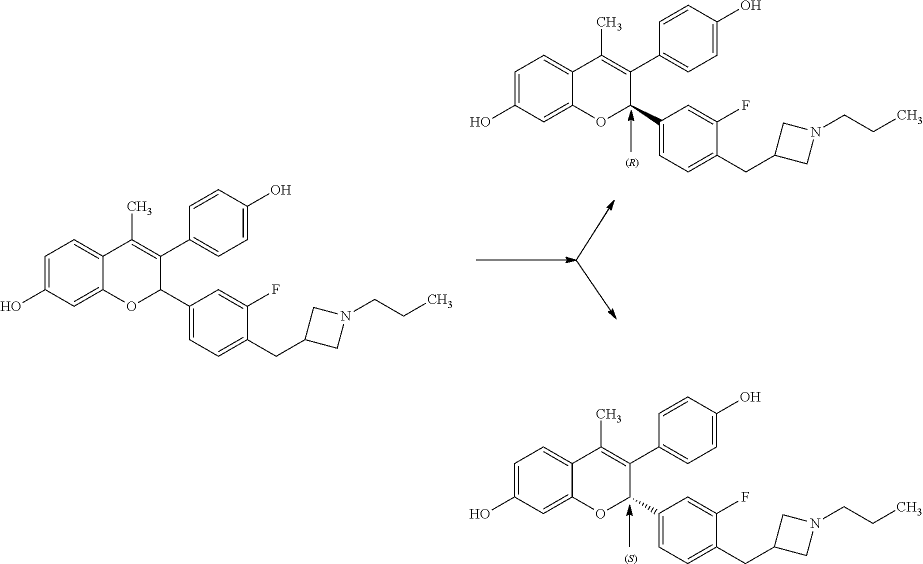

2-(3-fluoro-4-((1-propylazetidin-3-yl)methyl)phenyl)-3-(4-hydroxyphenyl)-4-methyl-2H-chromen-7-ol was dissolved into 2 mL of absolute ethanol. The solution was purified by preparative chromatography 500 μL injections over 4 runs. Fractions of each peak were pooled and concentrated separately. The solids were dried in a vacuum oven at 50° C. overnight. Peak 1, Compound 112: 45.4 mg, Peak 2, Compound 113: 40.7 mg.

Analytical HPLC

Column: ChiralPak AD-H, 250×4.6 mm

Temperature: 25° C.

Flow: 1 mL/min

Solvent system: 10% denatured EtOH (90% EtOH, 5% IPA, 5% MeOH) in Hex with 0.1% DEA

Purification on Preparative HPLC

Column: ChiralPak AD-H, 250×20 mm

Temperature: not regulated

Flow: 15 mL/min

Solvent system: 10% denatured EtOH (90% EtOH, 5% IPA, 5% MeOH) in Hex with 0.1% DEA

Chiral Retention Times (minutes)

Peak 1, Compound 112: 12.54

Peak 2, Compound 113: 17.11

Analytical Data

Peak 1, Compound 112

HPLC: 95.6%

ee: 100%

LCMS: [M+1]⁺, 460.3

¹H NMR (300 MHz, DMSO-d₆) δ 7.13-7.07 (m, 4H), 7.03-6.95 (m, 2H), 6.73 (d, J=8.7 Hz, 2H), 6.34 (dd, J=8.1, 2.4 Hz, 1H), 6.12 (d, J=2.1 Hz, 1H), 5.97 (s, 1H), 3.19-3.14 (m, 2H), 2.72-2.64 (m, 4H), 2.22 (t, J=7.8 Hz, 2H), 2.02 (s, 3H), 1.26-1.14 (m, 2H), 1.05-0.97 (m, 1H), 0.78 (t, J=7.5 Hz, 3H).

Peak 2, Compound 113

HPLC: 99.33%

ee: 100%

LCMS: [M+1]⁺, 460.3

¹H NMR (300 MHz, DMSO-d₆) δ: same as above.

### Example 16: Preparation of 5-fluoro-3-(4-hydroxyphenyl)-4-methyl-2-(4-((1-propylazetidin-3-yl)methyl)phenyl)-2H-chromen-7-ol

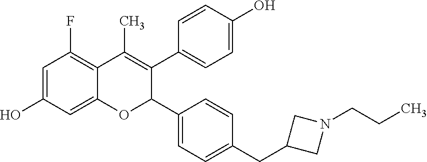 [SMILES: CCCN1CC(Cc2ccc(C3Oc4cc(O)cc(F)c4C(C)=C3c3ccc(O)cc3)cc2)C1]

### Step 1: Preparation of ethyl 3-(2-fluoro-4,6-dimethoxyphenyl)-2-(4-methoxyphenyl)-3-oxopropanoate

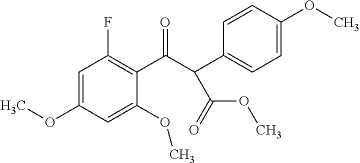

Thionyl chloride (9.1 mL, 125.0 mmol, 5.0 equiv.) was slowly added to a mixture of 2-fluoro-4,6-dimethoxybenzoic acid (5.000 g, 25.0 mmol, 1.0 equiv.) in anhydrous DCM (78 mL) at 0° C. followed by N,N-dimethylformamide (0.104 mL). The resulting mixture was stirred at RT for 2 h and concentrated in vacuo to provide the acid chloride.

To a solution of methyl 2-(4-methoxyphenyl)acetate (4.546 g, 25.2 mmol, 1.0 equiv.) in anhydrous THF (41.7 mL) at −78° C. was added lithium hexamethyl disilazide (37.5 mL, 37.5 mmol, 1.5 equiv.) dropwise using a pressure equalizing addition funnel. The resulting solution was stirred at −78° C. for 30 min, and a solution of the acid chloride in anhydrous THF (62.5 mL) was added dropwise through the same addition funnel (rinse with THF before adding the acid chloride solution). The reaction mixture was stirred for additional 2 h at −78° C. LCMS analysis showed a little starting material and product mass. The reaction was quenched with saturated NH₄Cl. The mixture was extracted twice with EA. The combined organic layers were dried over MgSO₄, filtered and concentrated under reduced pressure. The material was used directly in the next step without purification.

LCMS: [M+1]⁺, 363.5.

### Step 2: Preparation of 1-(2-fluoro-4,6-dimethoxyphenyl)-2-(4-methoxyphenyl)ethanone

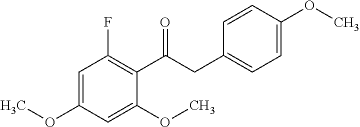

A mixture of crude ethyl 3-(2-fluoro-4,6-dimethoxyphenyl)-2-(4-methoxyphenyl)-3-oxopropanoate (11.770 g, 31.3 mmol, 1.0 equiv.) in DMSO (113 mL) and brine (11 mL) was refluxed at 150° C. for 5 h. After cooling to RT, the reaction mixture was diluted with water (3 times volume of DMSO) and extracted three times with EA. The combined organic layer was washed with brine, dried over MgSO₄, filtered, and concentrated. Purification by silica gel column chromatography (0-20% EA/Hex) provided the title compound as a liquid which solidified on standing to afford a pale yellow solid (5.57 g, 58.5%). NMR analysis shows the product with ˜91% purity.

¹H NMR (300 MHz, CDCl₃) δ 7.11 (dd, J=4.1, 1.8 Hz, 2H), 6.81 (dd, J=4.1, 1.8 Hz, 2H), 6.17 (d, J=2.4 Hz, 1H), 6.21 (d, J=2.4 Hz, 1H), 4.03 (s, 2H), 3.78 (s, 3H), 3.76 (s, 3H), 3.76 (s, 3H).

### Step 3: Preparation of 1-(2-fluoro-4,6-dihydroxyphenyl)-2-(4-hydroxyphenyl)ethanone

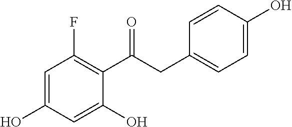 [SMILES: O=C(Cc1ccc(O)cc1)c1c(O)cc(O)cc1F]

1-(2-Fluoro-4,6-dimethoxyphenyl)-2-(4-methoxyphenyl)ethanone (2.770 g, 10.0 mmol, 1.0 equiv.) and pyridine hydrochloride (13.904 g, 120.3 mmol, 12.0 equiv.) were heated at 180° C. for 1 h under a stream of nitrogen. After cooling to RT, TLC showed starting material was still present. Additional pyridine hydrochloride (14 g) was added to the reaction and heated for another hour. TLC showed that additional product had formed but that starting material was still present. Additional pyridine hydrochloride (14 g) was added and allowed to react for 1 h. TLC showed much less starting material than product. A white solid (pyridine hydrochloride) collected on the sides of the flask above the oil bath. The reaction was cooled, then water (100 mL) and EA were added to the mixture to provide a black biphasic mixture. The layers were separated and the aqueous layer was extracted with EA (2×150 mL). The combined organic layer was washed with water (2×100 mL), dried over anhydrous magnesium sulfate, filtered and concentrated to a solid. Purification using flash chromatography (adsorbed to silica, 20-40% EA/Hex) provided the desired product as a solid (1.92 g, 73%).

LCMS: [M+1]⁺, 263.2.

¹H NMR (300 MHz, DMSO-d₆) δ 12.73 (s, 1H), 10.96 (s, 1H), 9.24 (s, 1H), 6.98 (d, J=8.2 Hz, 2H), 6.67 (d, J=8.4 Hz, 2H), 6.19-6.11 (m, 2H), 4.08 (s, 2H).

### Step 4: Preparation of 1-(2-fluoro-6-hydroxy-4-((tetrahydro-2H-pyran-2-yl)oxy)phenyl)-2-(4-((tetrahydro-2H-pyran-2-yl)oxy)phenyl)ethanone

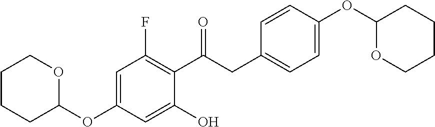 [SMILES: O=C(Cc1ccc(OC2CCCCO2)cc1)c1c(O)cc(OC2CCCCO2)cc1F]

1-(2-fluoro-4,6-dihydroxyphenyl)-2-(4-hydroxyphenyl)ethanone (2.750 g, 10.5 mmol, 1.0 equiv.), 3,4-dihydro-2H-pyran (4.3 mL, 47.2 mmol, 4.5 equiv.) and pyridinium para-toluene sulfonate (0.079 g, 0.3 mmol, 0.006 equiv) were suspended into EA (12.7 mL) and stirred at RT for 16 h. TLC (20% EA/Hex) showed two less polar spots and the starting material. More pyridinium para-toluene sulfonate was added and the reaction was stirred overnight. TLC showed less starting material than before. The reaction was stirred for another day with additional pyridinium para-toluene sulfonate. TLC showed that starting material was still present. The reaction was quenched with DIPEA (0.5 mL/0.27 g pyridinium para-toluene sulfonate), and concentrated to a yellow oil. The residue was purified using flash silica chromatography with 0-20% EA/Hex. The first peak was the desired product and fractions were concentrated to provide an oil (0.76 g, 16.8%).

¹H NMR (300 MHz, CDCl₃) δ 13.06 (s, 1H), 7.17 (d, J=8.7 Hz, 2H), 7.04-6.99 (m, 2H), 6.43-6.42 (m, 1H), 6.34 (dd, J=2.4, 14.1 Hz, 1H), 5.46 (t, J=3.6 Hz, 1H), 5.41 (t, J=3.6 Hz, 1H), 4.21 (d, J=3.6 Hz, 2H), 3.95-3.75 (m, 2H), 3.65-3.52 (m, 2H), 2.03-1.55 (m, 12H).

### Step 5: Preparation of 5-fluoro-2-(4-((1-propylazetidin-3-yl)methyl)phenyl)-7-((tetrahydro-2H-pyran-2-yl)oxy)-3-(4-((tetrahydro-2H-pyran-2-yl)oxy)phenyl)chroman-4-one

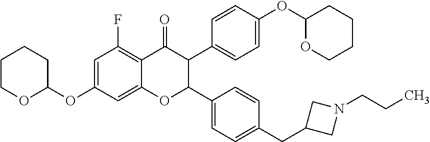 [SMILES: CCCN1CC(Cc2ccc(C3Oc4cc(OC5CCCCO5)cc(F)c4C(=O)C3c3ccc(OC4CCCCO4)cc3)cc2)C1]

1-(2-fluoro-6-hydroxy-4-((tetrahydro-2H-pyran-2-yl)oxy)phenyl)-2-(4-((tetrahydro-2H-pyran-2-yl)oxy)phenyl)ethanone (0.230 g, 0.5 mmol, 1.0 equiv.) was added to a 100 mL two-neck flask. 2-Butanol (5.4 mL) and 4-((1-propylazetidin-3-yl)methyl)benzaldehyde (0.124 g, 0.6 mmol, 1.1 equiv.) was added to the flask to give a suspension. Piperidine (0.017 mL, 0.2 mmol, 0.3 equiv.) and 1,8-diazabicyclo[5.4.0]undec-7-ene (0.081 mL, 0.5 mmol, 1.0 equiv.) was added to the mixture to give a suspension. The flask was fitted with a Dean-Stark apparatus and condenser and heated in an oil bath at 130° C. The white suspension became a light yellow solution. Half the solvent (2.7 mL) was collected over 20 min. The reaction was heated for an additional 12 h. LCMS indicated [M−18]⁺ and [M−1THP]⁺. TLC (5% MeOH/DCM) indicated that all the aldehyde was consumed and that there were two major spots. The desired product was less polar than the aldehyde and the other spot was more polar than the aldehyde. The reaction was cooled to RT and concentrated in vacuo at 30° C. The resulting residue was dissolved in DCM and loaded onto a silica gel column and purified (40 g, 0-15% MeOH/DCM). Flash chromatography did not show any UV absorbance at any of the monitored wavelengths (220, 254, and 280 nm). Fractions with a yellow color were collected (120 mg, 35.7%) and LCMS indicated [M-18]⁺ and [M-1THP]⁺. This material was used in the next reaction.

### Step 6: Preparation of 5-fluoro-3-(4-hydroxyphenyl)-4-methyl-2-(4-((1-propylazetidin-3-yl)methyl)phenyl)-2H-chromen-7-ol

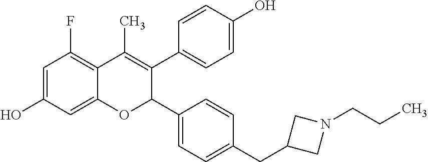 [SMILES: CCCN1CC(Cc2ccc(C3Oc4cc(O)cc(F)c4C(C)=C3c3ccc(O)cc3)cc2)C1]

This reaction was carried out in a manner substantially similar to that described in Steps 2 and 3 of Example 1.

### Example 17: Preparation of 5-fluoro-3-(4-hydroxyphenyl)-4-methyl-2-(4-((1-propylazetidin-3-yl)oxy)phenyl)2H-chromen-7-ol

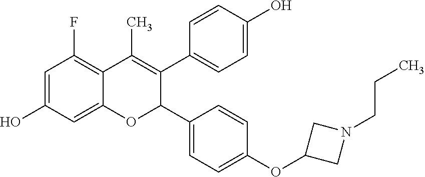 [SMILES: CCCN1CC(Oc2ccc(C3Oc4cc(O)cc(F)c4C(C)=C3c3ccc(O)cc3)cc2)C1]

### Step 1: Preparation of 5-fluoro-2-(4-((1-propylazetidin-3-yl)methyl)phenyl)-7-((tetrahydr-2H-pyran-2-yl)oxy)-3-(4-((tetrahydro-2H-pyran-2-yl)oxy)phenyl)chroman-4-one

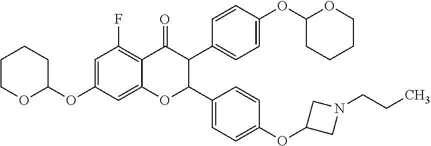 [SMILES: CCCN1CC(Oc2ccc(C3Oc4cc(OC5CCCCO5)cc(F)c4C(=O)C3c3ccc(OC4CCCCO4)cc3)cc2)C1]

1-(2-fluoro-6-hydroxy-4-((tetrahydro-2H-pyran-2-yl)oxy)phenyl)-2-(4-((tetrahydro-2H-pyran-2-yl)oxy)phenyl)ethanone (0.363 g, 0.8 mmol, 1.1 equiv.) was added to a 20 mL flask. 2-Butanol (8.4 mL) and 4-((1-propylazetidin-3-yl)oxy)benzaldehyde (0.176 g, 0.8 mmol, 1.0 equiv.) were added to the flask to give a suspension. Piperidine (0.026 mL, 0.3 mmol, 0.3 equiv.) and 1,8-diazabicyclo[5.4.0]undec-7-ene (0.121 mL, 0.8 mmol, 1.0 equiv.) were added to the mixture to give a yellow suspension. The flask was fitted with a Dean-Stark apparatus and condenser and heated in an oil bath at 130° C. The suspension became a light yellow solution. Half the solvent (4.2 mL) was collected over 20 min. The reaction was heated for an additional 12 h. LCMS indicated the presence of [M+1]⁺ and [M−THP]⁺. The reaction was cooled to RT and concentrated in vacuo. The resulting residue was dissolved in DCM and loaded to a silica gel column and purified (12 g, 0-15% MeOH/DCM). TLC (5% MeOH/DCM) indicated there was a spot that had the same R_{f} as starting aldehyde but LCMS indicated that it was product (330 mg). A second spot more polar than product with unknown impurities was the mono-protected product according to LCMS. The third spot, the most polar one (128 mg), was also a mono-protected product according to LCMS.

### Step 2: Preparation of 5-fluoro-3-(4-hydroxyphenyl)-4-methyl-2-(4-((1-propylazetidin-3-yl)oxy)phenyl)-2-chromen-7-ol

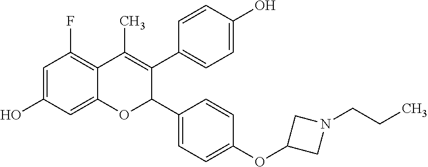 [SMILES: CCCN1CC(Oc2ccc(C3Oc4cc(O)cc(F)c4C(C)=C3c3ccc(O)cc3)cc2)C1]

The Grignard addition and dehydration was carried out in a manner substantially similar to that used in Preparation 2.

### Example 18: Preparation of 4-methyl-3-phenyl-2-(4-((1-propylazetidin-3-yl)oxy)phenyl)-2H-chromen-7-ol (Compound 114)

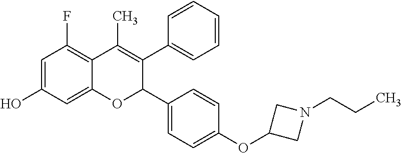 [SMILES: CCCN1CC(Oc2ccc(C3Oc4cc(O)cc(F)c4C(C)=C3c3ccccc3)cc2)C1]

### Step 1: Preparation of 1-(2,4-dihydroxyphenyl)-2-phenylethanone

Resorcinol (1,3-dihydroxybenzene) (8.190 g, 74.4 mmol, 1.0 equiv.) and phenylacetic acid (10.430 g, 76.6 mmol, 1.0 equiv.) were added to a 250 mL round bottomed flask fitted with a stir bar and a condenser. Toluene (36.514 mL) was added to the flask to afford a suspension. Boron trifluoride etherate (26.182 mL, 208.5 mmol, 2.8 equiv.) was added through a syringe. The reaction was stirred and heated slowly to 100° C. The suspension became a light orange solution at 100° C. The reaction was stirred at the same temperature for 2 h. TLC (30% EA/Hex) indicated the reaction was complete. The reaction was cooled to ambient temperature. A 12% aqueous solution of sodium acetate (0.645 g, 55 mL) was added dropwise to the reaction mixture and the reaction was stirred at RT for 2 h. The mixture was diluted with EA and the organic layer was washed with 12% sodium acetate aqueous solution (60 mL), brine, dried over anhydrous sodium sulfate, filtered and concentrated. The red oil was added to deionized water and heated at 70° C. for 30 min under nitrogen. The oil slowly solidified to afford a tan solid. The mixture was cooled to ambient temperature and then to 0° C. The solids were collected and rinsed with water. The solids were dissolved in EA and dried over anhydrous sodium sulfate, filtered and concentrated. The resulting solid was used directly in the next step without purification.

¹H NMR (300 MHz, DMSO-d₆) δ 12.51 (s, 1H), 10.67 (s, 1H), 7.94 (d, J=8.7 Hz, 1H), 7.34-7.21 (m, 5H), 6.41-6.37 (m, 1H), 6.26 (d, J=2.1 Hz, 1H), 4.29 (s, 2H).

### Step 2: Preparation of 1-(2-hydroxy-4-((tetrahydro-2H-pyran-2-yl)oxy)phenyl)-2-phenylethanone

1-(2,4-Dihydroxyphenyl)-2-phenylethanone (17.000 g, 74.5 mmol, 1.0 equiv.) and ethyl acetate (600 mL) were added to a three necked round bottomed flask (500 mL) equipped with a stir bar, a thermometer and a nitrogen balloon. The flask was evacuated and purged with nitrogen. 3,4-Dihydro-2H-pyran (9.0 mL, 98.6 mmol, 1.3 equiv.) and p-toluenesulfonic acid (1 mg) were added to the reaction. The solution warmed slightly. The solution was stirred at RT overnight. TLC (20% EA/Hex) indicated there were two equal amounts of products less polar than the starting material and starting material present. DHP (8 mL) and a catalytic amount of pyridinium p-toluenesulfonate were added at RT and the reaction was stirred for an additional 12 h. TLC (20% EA/Hex) indicated that the reaction was complete. TEA was added and the reaction mixture was concentrated to a brown residue. The residue was treated with MeOH and IPA but no solids were generated. The residue was dissolved in DCM and loaded onto a silica gel column (300 g, 0-20% EA/Hex). Fractions containing the product and small amounts of impurities were combined and concentrated. After concentration, the resulting residue was diluted with MeOH, the solid was filtered, and washed with MeOH to afford the title compound as a white solid (9 g). The filtrate was concentrated to afford additional product as a pale yellow solid (5 g).

¹H NMR (300 MHz, CDCl₃) δ 12.57 (s, 1H), 7.76 (d, J=9.0 Hz, 1H), 7.37-7.24 (m, 5H), 6.62 (d, J=2.4 Hz, 1H), 6.56 (dd, J=8.7, 2.4 Hz, 1H), 5.48 (t, J=2.8 Hz, 1H), 4.22 (s, 2H), 3.87-3.79 (m, 1H), 3.65-3.60 (m, 1H), 2.01-1.88 (m, 1H), 1.87-1.83 (m, 2H), 1.74-1.57 (m, 3H).

### Step 3: Preparation of 3-phenyl-2-(4-((1-propylazetidin-3-yl)oxy)phenyl)-7-((tetrahydro-2H-pyran-2-yl)oxy)chroman-4-one

1-(2-hydroxy-4-((tetrahydro-2H-pyran-2-yl)oxy)phenyl)-2-phenylethanone (0.780 g, 2.5 mmol, 1.1 equiv.), 2-butanol (22.0 ml), 4-((1-propylazetidin-3-yl)oxy)benzaldehyde (0.50 g, 2.3 mmol, 1.0 equiv.), piperidine (0.224 mL, 2.3 mmol, 1.0 equiv.) and 1,8-diazabicyclo[5.4.0]undec-7-ene (0.224 mL, 1.5 mmol, 0.7 equiv.) were added sequentially to a 100 mL round bottom flask. The flask was fitted with a Dean-Stark apparatus and condenser and heated in an oil bath at 130° C. Half the solvent (11 mL) was collected over 30 min. The reaction was heated for a further 12 h. LCMS indicated that the desired product was present. The reaction was concentrated and dissolved in DCM and loaded onto a silica gel column (25 g, 0-10% MeOH/DCM) to afford the title compound as a light brown foam. This material was used without further purification.

LCMS, [M+1]+, 514.2.

Step 4: Preparation of 4-methyl-3-phenyl-2-(4-((1-propylazetidin-3-yl)oxy)phenyl)-2H-chromen-7-ol

To a solution of 90.0% 3-phenyl-2-(4-((1-propylazetidin-3-yl)oxy)phenyl)-7-((tetrahydro-2H-pyran-2-yl)oxy)chroman-4-one (0.229 g, 0.4 mmol, 1.0 equiv.) in THF (0.003 L, 1.2 mmol, 3.7 equiv.) at 0° C. was added methylmagnesium chloride (3.0 M solution in THF, 0.544 mL, 1.6 mmol, 5.0 equiv.) dropwise. After complete addition of the Grignard reagent, the reaction was removed from the ice-bath and allowed to reach RT and stirred for 12 h. LCMS indicated the reaction was complete and the desired mass [M+1]⁺, 530.2 was observed. The reaction mixture was cooled to 0° C. and quenched with saturated aq. ammonium chloride (2 mL). The ice-bath was removed and the resulting suspension was stirred at RT for 15 min before being filtered through a pad of Celite®. The filtrate was dried over anhydrous sodium sulfate, filtered and concentrated. The resulting residue (pale yellow foam) was added to 80% acetic acid/H₂O (10 mL) and evacuated and purged with nitrogen. The solution was heated at 90° C. for 40 min. HPLC indicated that the reaction was complete and LCMS indicated the product was present. The reaction mixture was concentrated and cooled to RT. The reaction mixture was diluted with saturated aq. sodium bicarbonate, EA and stirred at RT for 30 min. The mixture was separated. The organic layer was washed with saturated aq. sodium bicarbonate, water, brine, dried over anhydrous sodium sulfate, filtered and concentrated. The resulting residue was dissolved in EA and loaded onto a silica gel column (25 g, 50% EA/Hex, 0-10% MeOH/DCM) and chromatographed to afford the title compound as an off-white foam. HPLC 94.4%.

LCMS, [M+1]⁺, 428.1.

¹H NMR (300 MHz, CDCl₃) δ 7.32-7.14 (m, 8H), 6.65 (d, J=8.1 Hz, 2H), 6.36 (dd, J=9.0, 2.4 Hz, 1H), 6.13 (d, J=2.4 Hz, 1H), 5.83 (s, 1H), 4.77-4.72 (m, 1H), 3.74 (t, J=7.4 Hz, 2H), 3.18-3.10 (m, 2H), 2.48 (t, J=7.5 Hz, 2H), 1.43-1.35 (m, 2H), 0.90 (t, J=7.5 Hz, 3H).

### Examples 19 and 20: Separation of 4-methyl-3-phenyl-2-(4-((1-propylazetidin-3-yl)oxy)phenyl)-2H-chromen-7-ol, S-Isomer (Compound 117) and R-Isomer (Compound 118)

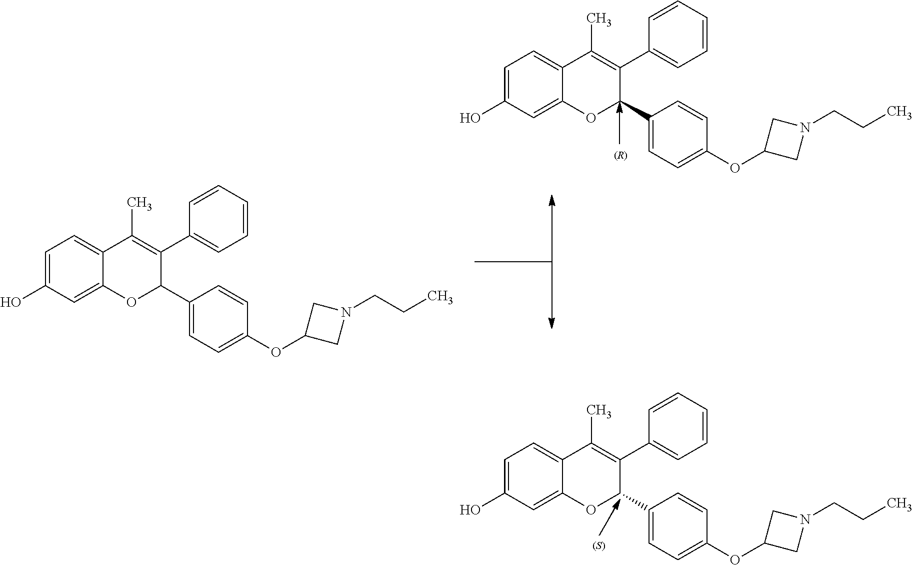

The enantiomers of Compound 114 were separated via chromatography using a Daicel OJ column and 99.9% EtOH w/0.1% DEA as mobile phase to afford peak 1, 31.5 mg and peak 2, 31.1 mg.

Analytical HPLC

Column: ChiralPak OJ-H, 5 micron, 4.6×260 mm

Temperature: Ambient

Flow: 0.5 mL/min.

Solvent System: 99.9% EtOH, 0.1% DEA.

Chiral Retention Times (minutes)

Peak 1, Compound 117 (S-isomer): 9.3

Peak 2, Compound 118 (R-isomer): 12.0

Analytical Data:

Peak 1, Compound 117

LCMS: [M+1], 428.1

¹H NMR (300 MHz, CDCl₃), δ 7.32-7.14 (m, 8H), 6.65 (d, J=8.1 Hz, 2H), 6.36 (dd, J=9.0, 2.4 Hz, 1H), 6.13 (d, J=2.4 Hz, 1H), 5.83 (s, 1H), 4.77-4.72 (m, 1H), 3.74 (t, J=7.4 Hz, 2H), 3.18-3.10 (m, 2H), 2.48 (t, J=7.5 Hz, 2H), 1.43-1.35 (m, 2H), 0.90 (t, J=7.5 Hz, 3H).

Peak 2, Compound 118

LCMS [M+1], 428.1

¹H NMR (300 MHz, CDCl₃), δ: same as above

### Example 21: Preparation of 3-(4-fluorophenyl)-4-methyl-2-(4-((1-propylazetidin-3-yl)oxy)phenyl-2H-chromen-7-ol (Compound 115)

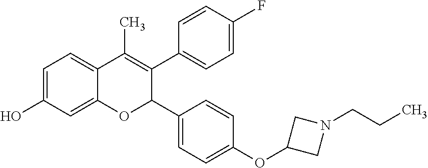 [SMILES: CCCN1CC(Oc2ccc(C3Oc4cc(O)ccc4C(C)=C3c3ccc(F)cc3)cc2)C1]

### Step 1. Preparation of 1-(2,4-dihydroxyphenyl)-2-(4-fluorophenyl)ethanone

To a suspension of resorcinol (1,3-dihydroxybenzene) (5.000 g, 45.4 mmol, 1.0 equiv.), 4-fluorophenylacetic acid (7.209 g, 46.8 mmol, 1.0 equiv.) and toluene (20.000 mL, 188.8 mmol, 4.2 equiv.) was added boron trifluoride etherate (15.97 mL, 127.1 mmol, 2.8 equiv.), and the suspension was heated at 100° C. to afford a clear orange-colored solution. After the reaction was stirred at 100° C. for 1 h, LCMS and TLC (EA:Hex=1:2) analysis indicated that the reaction was complete. The reaction was cooled to RT and slowly quenched with a 12% aqueous solution of sodium acetate (100 mL) and allowed to stir for 2 h. Note: The quench should be carefully monitored as the reaction is exothermic. The orange-colored solution was diluted with EA, the layers separated, and the organic layer washed with a 12% aqueous solution of sodium acetate (1×50 mL), brine (1×100 mL), dried over anhydrous Na₂SO₄, and concentrated to dryness. A NMR analysis (¹H and ¹⁹F) of the crude product indicated the presence of both of the phenolic protons that was confirmed by a D₂O shake for the hydrogen-deuterium exchange. Crude residue was triturated from EA/Hex to provide 1-(2,4-dihydroxyphenyl)-2-(4-fluorophenyl)ethanone (8.34 g, 75%) as a pale orange solid.

¹H NMR (300 MHz, DMSO-ds) δ 12.4 (s, 1H), 10.7 (s, 1H), 7.91 (d, J=9.0 Hz, 1H), 7.32-7.28 (m, 2H), 7.15-7.09 (m, 2H), 6.37 (dd, J=9.0, 2.4 Hz, 1H), 6.24 (d, J=2.4 Hz, 1H), 4.30 (s, 2H). ¹⁹F NMR (282 MHz, DMSO-d₆) δ 116.6-116.5 (m, 1F). m/z=245 (M−H)+.

### Step 2. Preparation of 2-(4-fluorophenyl)-1-(2-hydroxy-4-((tetrahydro-2H-pyran-2-yl)oxy)phenyl)ethanone

A 1 L round bottom flask under argon, was charged with 1-(2,4-dihydroxyphenyl)-2-(4-fluorophenyl)ethanone (8.340 g, 33.9 mmol, 1.0 equiv.) and pyridinium p-toluenesulfonate (1.607 g, 6.8 mmol, 0.2 equiv.) in dichloromethane (250.0 mL, 2360.6 mmol, 69.7 equiv.). 3,4-Dihydro-2H-pyran (6.18 mL, 67.7 mmol, 2.0 equiv.) was slowly added and the reaction was allowed to stir at RT over 3 days. The reaction mixture was washed with saturated sodium bicarbonate (2×200 mL), brine (1×100 mL), dried over anhydrous Na₂SO₄, and concentrated to dryness. The crude residue was adsorbed onto silica gel, and purified by flash chromatography. The column was eluted with MeOH (0 to 3%) in DCM to provide the title compound (8.97 g, 80%) as an off-white solid.

¹H NMR (300 MHz, DMSO-d) δ 12.3 (s, 1H), 7.99 (d, J=9.3 Hz, 1H), 7.33-7.28 (m, 2H), 7.17-7.10 (m, 2H), 6.59 (dd, J=8.7, 2.4 Hz, 1H), 6.54 (d, J=2.4 Hz, 1H), 5.59 (t, J=3.3 Hz, 1H), 4.35 (s, 2H), 3.75-3.50 (m, 2H), 1.90-1.50 (m, 6H). ¹⁹F NMR (282 MHz, DMSO-d₆) δ ppm 116.5-116.4 (m, 1F). m/z=329 (M−H)+.

### Step 3. Preparation of 3-(4-fluorophenyl)-2-(4-((1-propylazetidin-3-yl)oxy)phenyl)-7-((tetrahydro-2H-pyran-2-yl)oxy)chroman-4-one

A mixture of 4-((1-propylazetidin-3-yl)oxy)benzaldehyde (1.220 g, 5.6 mmol, 1.0 equiv.), 2-(4-fluorophenyl)-1-(2-hydroxy-4-((tetrahydro-2H-pyran-2-yl)oxy)phenyl)ethanone (2.022 g, 6.1 mmol, 1.1 equiv.), piperidine (0.550 mL, 5.6 mmol, 1.0 equiv.) and 1,8-diazabicyclo[5.4.0]undec-7-ene (0.549 mL, 3.7 mmol, 0.7 equiv.) in 2-butanol (50.000 mL) in a round bottom flask equipped with a Dean-Stark apparatus and condenser were heated at 130° C. Half the volume (25 mL) was collected over 2 h, and the reaction was further heated at 130° C. overnight. TLC (DCM:MeOH=9:1) showed two overlapping spots while LCMS indicated similar masses for both the major (cyclized) and minor (uncyclized) products. The reaction mixture was allowed to cool to RT and concentrated to dryness. The crude residue was adsorbed onto silica gel and purified by combiflash column chromatography. The column was eluted with MeOH (0 to 20%) in DCM to provide a mixture of the minor uncyclized (HPLC 16%) and desired major cyclized (HPLC 84%) product, 3-(4-fluorophenyl)-2-(4-((1-propylazetidin-3-yl)oxy)phenyl)-7-((tetrahydro-2H-pyran-2-yl)oxy)chroman-4-one (2.06 g, 70%), as a light-brown foam.

¹H NMR (300 MHz, DMSO-d₆) δ 7.71 (d, J=8.7 Hz, 1H), 7.27 (d, J=8.4 Hz, 1H), 7.12-6.61 (m, 9H), 5.78 (d, J=7.2 Hz, 1H), 5.60-5.55 (m, 1H), 5.47 (d, J=6.9 Hz, 1H), 4.65 (pent, J=5.6 Hz, 1H), 3.72-3.63 (m, 4H), 2.90-2.84 (m, 2H), 2.38-2.33 (m, 2H), 1.85-1.50 (m, 6H), 1.30-1.21 (m, 2H), 0.780 (t, J=7.2 Hz, 3H). ¹⁹F NMR (282 MHz, DMSO-d₆) δ 115.9 (s, 1F). m/z=532 (M+H)+.

### Step 4. Preparation of 3-(4-fluorophenyl)-4-methyl-2-(4-((1-propylazetidin-3-yl)oxy)phenyl)-2H-chromen-7-ol

To a solution of 3-(4-fluorophenyl)-2-(4-((1-propylazetidin-3-yl)oxy)phenyl)-7-((tetrahydro-2H-pyran-2-yl)oxy)chroman-4-one (1.030 g, 1.9 mmol, 1.0 equiv.) in tetrahydrofuran (15.0 mL, 151.9 mmol, 78.4 equiv.) at 0° C. was added methylmagnesium bromide in THF 3M (3.23 mL, 9.7 mmol, 5.0 equiv.) dropwise. The ice bath was removed, and the reaction mixture was allowed to warm to RT overnight. LCMS indicated that the reaction was complete and afforded the desired mass of the tertiary alcohol (m/z=548). The reaction mixture was cooled to 0° C. and quenched with saturated aqueous ammonium chloride (10 mL). The ice-bath was removed, and the resulting suspension was stirred at RT for 30 min affording a solution. The solution was diluted with EA, and the layers were separated. The aqueous layer was extracted with EA (1×50 mL). The combined organics were dried over anhydrous. Na₂SO₄, and concentrated to dryness. To the resulting crude pale-yellow foam (1.06 g), was added 10 mL of an 80% acetic acid/H₂O solution, and the yellow solution was purged with nitrogen and heated at 90° C. for 45 min. LCMS indicated that the reaction was complete. The reaction mixture was concentrated to dryness on a rotovap under high vacuum with a water bath at 40° C. Before releasing the flask from the rotovap, the rotovap was charged with nitrogen. The reaction mixture was charged with saturated sodium bicarbonate (100 mL) and EA (50 mL) and stirred at RT for 30 min. The layers were separated, and the organic layer washed with saturated sodium bicarbonate (2×50 mL), brine (1×100 mL), dried over anhydrous Na₂SO₄, and concentrated to dryness. The crude residue was adsorbed on silica gel, and purified by flash chromatography. The silica gel column was eluted with MeOH (0 to 20%) in DCM to provide the title compound (205 mg, 24%) as a light-brown foam (HPLC purity 97%) and 220 mg of mixed fractions with 88% purity by HPLC.

¹H NMR (300 MHz, DMSO-d₆) δ 9.49 (s, 1H), 7.29-7.11 (m, 6H), 6.66 (d, J=9.0 Hz, 2H), 6.31 (dd, J=8.7, 3.0 Hz, 1H), 6.07 (d, J=2.4 Hz, 1H), 5.93 (s, 1H), 4.60 (pent, J=5.6 Hz, 1H), 3.68-3.63 (m, 2H), 2.82 (t, J=5.1 Hz, 2H), 2.31 (t, J=6.9 Hz, 2H), 1.99 (s, 3H), 1.31-1.19 (m, 2H), 0.776 (t, J=6.9 Hz, 3H). ¹⁹F NMR (282 MHz, DMSO-d₆) δ 115.0-114.9 (m, 1F). m/z=447 (M+H)+.

The enantiomers are separated in a similar manner as described above.

### Example 22: Preparation of 3-(2-chloro-4-fluorophenyl)-4-methyl-2-(4-((1-propylazetidin-3-yl)oxy)phenyl)-2H-chromen-7-ol (Compound 116)

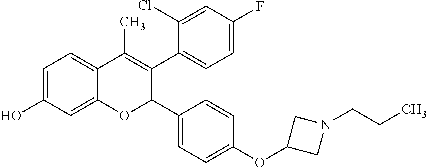 [SMILES: CCCN1CC(Oc2ccc(C3Oc4cc(O)ccc4C(C)=C3c3ccc(F)cc3Cl)cc2)C1]

### Step 1: Preparation of 2-(2-chloro-4-fluorophenyl)-1-(2,4-dihydroxyphenyl)ethanone

To a suspension of resorcinol (1,3-dihydroxybenzene) (5.000 g, 45.4 mmol, 1.0 equiv.), 2-chloro-4-fluorophenylacetic acid (8.820 g, 46.8 mmol, 1.0 equiv.) in toluene (20.000 mL, 188.8 mmol, 4.2 equiv.) was added boron trifluoride etherate (15.969 mL, 127.1 mmol, 2.8 equiv.), and the suspension was heated at 100° C. to afford a burgundy red solution. After the reaction was stirred at 100° C. for 1 h, LCMS and TLC (EA:Hex=1:2) analysis indicated that the reaction was complete. The reaction was cooled to RT and slowly quenched with a 12% aqueous solution of sodium acetate (100 mL) and allowed to stir for 2 h. Note: The quench should be carefully monitored as the reaction is exothermic. The red-colored solution was diluted with EA, and the layers separated. The organic layer was washed with a 12% aqueous solution of sodium acetate (1×50 mL), brine (1×100 mL), dried over anhydrous Na₂SO₄, and concentrated to dryness. NMR analysis (‘H and’⁹F) of the crude oily product indicated the presence of both the phenolic protons. This was confirmed by an hydrogen/deuterium exchange with D₂O. The crude product was adsorbed on silica gel, and purified by combiflash column chromatography. The column was eluted with MeOH (0 to 5%) in DCM to provide a yellow solid. Trituration of the chromatographed product with EA/Hex provided the title compound (4.10 g, 32%) as an off-white solid.

¹H NMR (300 MHz, DMSO-d₆) δ 12.1 (s, 1H), 10.6 (s, 1H), 7.91 (d, J=9.3 Hz, 1H), 7.45-7.39 (m, 2H), 7.21-7.15 (m, 1H), 6.42-6.37 (m, 1H), 6.25 (d, J=2.4 Hz, 1H), 4.45 (s, 2H). ¹⁹F NMR (282 MHz, DMSO-d₆) δ-113.8-113.6 (m, 1F). m/z=279 (M−H)+.

### Step 2: Preparation of 2-(2-chloro-4-fluorophenyl)-1-(2-hydroxy-4-((tetrahydro-2H-pyran-2-yl)oxy)phenyl)ethanone

To a 500 mL round bottom flask under argon was added a mixture of 2-(2-chloro-4-fluorophenyl)-1-(2,4-dihydroxyphenyl)ethanone (4.100 g, 14.6 mmol, 1.0 equiv.) and pyridinium p-toluenesulfonate (0.693 g, 2.9 mmol, 0.2 equiv.) in DCM (120.0 mL, 1133.1 mmol, 77.6 equiv.). The compound 3,4-dihydro-2H-pyran (2.665 ml, 29.2 mmol, 2.0 equiv.) was slowly added and the reaction was allowed to stir at RT over 2 days. The reaction mixture was washed with saturated sodium bicarbonate (2×100 mL), brine (1×100 mL), dried over anhydrous Na₂SO₄, and concentrated to dryness. The crude residue was adsorbed on silica gel, and purified by flash chromatography. The column was eluted with MeOH (0 to 2%) in DCM to provide 2-(2-chloro-4-fluorophenyl)-1-(2-hydroxy-4-((tetrahydro-2H-pyran-2-yl)oxy)phenyl)ethanone (4.34 g, 81%) as an off-white solid.

¹H NMR (300 MHz, DMSO-d₆) δ 12.0 (s, 1H), 7.99 (d, J=8.7 Hz, 1H), 7.46-7.39 (m, 2H), 7.19-7.15 (m, 1H), 6.61 (dd, J=8.7, 2.4 Hz, 1H), 6.55 (d, J=2.4 Hz, 1H), 5.62-5.58 (m, 1H), 4.52 (s, 2H), 3.73-3.55 (m, 2H), 1.87-1.47 (m, 6H). ¹⁹F NMR (282 MHz, DMSO-d₆) δ 113.7-113.6 (m, 1F). m/z=365 (M+H)+

### Step 3: Preparation of 3-(2-chloro-4-fluorophenyl)-2-(4-((1-propylazetidin-3-yl)oxy)phenyl)-7-((tetrahydro-2H-pyran-2-yl)oxy)chroman-4-one

A mixture of 4-((1-propylazetidin-3-yl)oxy)benzaldehyde (1.220 g, 5.6 mmol, 1.0 equiv.), 2-(2-chloro-4-fluorophenyl)-1-(2-hydroxy-4-((tetrahydro-2H-pyran-2-yl)oxy)phenyl)ethanone (2.022 g, 5.5 mmol, 1.1 equiv.), piperidine (0.550 mL, 5.6 mmol, 1.0 equiv.) and 1,8-diazabicyclo[5.4.0]undec-7-ene (0.549 mL, 3.7 mmol, 0.7 equiv.) in 2-butanol (50.0 mL) in a round bottom flask equipped with a Dean-Stark apparatus and condenser were heated at 130° C. Half the volume (25 mL) was collected over 2 h, and the reaction was further heated overnight at 130° C. TLC (DCM:MeOH=9:1) showed two overlapping spots while LCMS indicated similar masses for both the major and minor products. The reaction mixture was allowed to cool to RT and concentrated to dryness. The crude residue was adsorbed on silica gel, and purified by flash chromatography. The column was eluted with MeOH (0 to 20%) in DCM to provide a mixture of the minor (HPLC 14%) and desired major (HPLC 86%) product, 3-(2-chloro-4-fluorophenyl)-2-(4-((1-propylazetidin-3-yl)oxy)phenyl)-7-((tetrahydro-2H-pyran-2-yl)oxy)chroman-4-one (2.06 g, 66%), as a light-brown foam.

¹H NMR (300 MHz, DMSO-d₆) δ 7.76 (d, J=8.7 Hz, 1H), 7.35-7.27 (m, 4H), 7.12-7.00 (m, 2H), 6.73-6.68 (m, 3H), 6.01-5.95 (m, 1H), 5.62-5.57 (m, 1H), 4.94-4.89 (m, 1H), 4.65 (pent, J=5.4 Hz, 1H), 3.73-3.64 (m, 4H), 2.87-2.83 (m, 2H), 2.38-2.33 (m, 2H), 1.85-1.50 (m, 6H), 1.33-1.21 (m, 2H), 0.776 (t, J=7.2 Hz, 3H). ¹⁹F NMR (282 MHz, DMSO-d₆) δ 113.05-113.0 (m, 1F). m/z=566 (M+H)+.

### Step 4: Preparation of 3-(2-chloro-4-fluorophenyl)-4-methyl-2-(4-((1-propylazetidin-3-yl)oxy)phenyl)-2H-chromen-7-ol

To a solution of 3-(2-chloro-4-fluorophenyl)-2-(4-((1-propylazetidin-3-yl)oxy)phenyl)-7-((tetrahydro-2H-pyran-2-yl)oxy)chroman-4-one (1.030 g, 1.8 mmol, 1.0 equiv.) in tetrahydrofuran (15.000 mL, 151.9 mmol, 78.4 equiv.) at 0° C. was added dropwise methylmagnesium bromide in THF 3M (3.229 mL, 9.7 mmol, 5.0 equiv.). The ice-bath was removed, and the reaction mixture was allowed to warm to RT overnight. LCMS indicated that the reaction was complete and afforded the desired mass of the tertiary alcohol (m/z=583). The reaction mixture was cooled back to 0° C. and quenched with saturated aqueous ammonium chloride (10 mL). The ice-bath was removed, and the resulting suspension was stirred at RT for 30 min affording a solution. The solution was diluted with EA, the layers were separated, and the aqueous layer was extracted with EA (1×50 mL). The combined organics were dried over anhydrous Na₂SO₄, and concentrated to dryness. To the resulting crude pale-yellow foam (1.07 g), was added 10 mL of an 80% acetic acid/H₂O solution, and the yellow solution was purged with nitrogen and heated at 90° C. for 45 min under nitrogen. LCMS indicated that the reaction was complete. The reaction mixture was concentrated to dryness on a rotary evaporator under high vacuum and the use of a water bath at 40° C. Before releasing the flask from the rotovap, the rotovap was filled with nitrogen. The reaction mixture was charged with saturated sodium bicarbonate (100 mL) and EA (50 mL) and stirred at RT for 30 min. The layers were separated, and the organic layer washed with saturated sodium bicarbonate (2×50 mL), brine (1×100 mL), dried over anhydrous Na₂SO₄, and concentrated to dryness. The crude residue was adsorbed on silica gel and purified by combiflash column chromatography (40 g column, 0-10% MeOH/DCM) to provide the title compound (75 mg, 9%) as a light-brown foam (HPLC purity 95%) and two sets of mixed fractions (mixed fraction-1, 315 mg 92% pure, and mixed fraction-2, 186 mg 85% pure by HPLC).

¹H NMR (300 MHz, DMSO-d₆) δ 9.58 (bs, 1H), 7.49 (dd, J=8.1, 2.1 Hz, 1H), 7.23-7.09 (m, 4H), 6.90-6.85 (m, 1H), 6.68 (d, J=8.7 Hz, 2H), 6.33 (dd, J=8.4, 2.4 Hz, 1H), 6.08 (d, J=1.8 Hz, 1H), 5.69 (s, 1H), 4.60 (pent, J=5.7 Hz, 1H), 3.73-3.60 (m, 2H), 2.87-2.80 (m, 2H), 2.31 (t, J=6.9 Hz, 2H), 1.79 (s, 3H), 1.31-1.21 (m, 2H), 0.780 (t, J=7.2 Hz, 3H). ¹⁹F NMR (282 MHz, DMSO-d₆) δ 112.4-112.3 (m, 1F).m/z=480 (M+H)+.

The enantiomers are separated in a similar manner as described above.

### Example 23: Preparation of 3-(2-isopropylphenyl)-4-methyl-2-(4-((1-propylazetidin-3-yl)oxy)phenyl)-2H-chromen-7-ol (Compound 119)

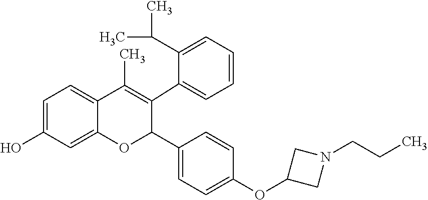 [SMILES: CCCN1CC(Oc2ccc(C3Oc4cc(O)ccc4C(C)=C3c3ccccc3C(C)C)cc2)C1]

### Step 1: Preparation of 1-(2,4-dihydroxyphenyl-2-(2-iso-propylphenyl)ethanone

To a suspension of resorcinol (1,3-dihydroxybenzene) (410 mg, 3.7 mmol, 1.0 equiv.), 2-(2-iso-propylphenyl)acetic acid (684 mg, 3.8 mmol, 1.0 equiv.) in toluene (6.000 mL, 56.7 mmol, 15.2 equiv.) was added boron trifluoride etherate (1.309 mL, 10.4 mmol, 2.8 equiv.).

The suspension was heated at 100° C. giving a yellowish-brown solution. After the reaction was stirred at 100° C. for 1 h, LCMS and TLC (DCM:MeOH=9:1) analysis indicated completion of the reaction. The reaction was cooled to RT and slowly quenched (small exotherm) with a 12% aqueous solution of sodium acetate (5 mL) and allowed to stir for 2 h. The yellowish mixture was diluted with EA, and the phases separated. The organic phases were washed with a 12% aqueous solution of sodium acetate (25 mL), brine (50 mL), dried over anhydrous Na₂SO₄, filtered, and concentrated to dryness. The crude product was adsorbed on silica gel and purified by Combiflash column chromatography (40 g column, 0-5% MeOH/DCM) to provide 1-(2,4-dihydroxyphenyl)-2-(2-iso-propylphenyl)ethanone (470 mg, 47%) as a yellow solid. ¹H-NMR (300 MHz, DMSO-d₆) δ ppm 12.4 (s, 1H), 10.6 (s, 1H), 7.95 (d, J=8.7 Hz, 1H), 7.30-7.08 (m, 4H), 6.38 (dd, J=8.7, 2.4 Hz, 1H), 6.25 (d, J=2.4 Hz, 1H), 4.90 (s, 2H), 3.04 (sept, J=7.2 Hz, 1H), 1.10 (d, J=6.3 Hz, 6H). m/z=271 (M+H)+.

### Step 2. Preparation of 1-(2-hydroxy-4-((tetrahydro-2H-pyran-2-yl)oxy)phenyl)-2-(2-iso-propylphenyl)ethanone

To a round bottom flask under argon, a mixture of l-(2,4-dihydroxyphenyl)-2-(2-iso-propylphenyl)ethanone (470 mg, 1.7 mmol, 1.0 equiv.) and pyridinium p-toluenesulfonate (83 mg, 0.3 mmol, 0.2 equiv.) in DCM (15.000 mL, 141.6 mmol, 81.5 equiv.) was slowly added 3,4-dihydro-2H-pyran (0.317 mL, 3.5 mmol, 2.0 equiv.). The mixture was stirred at RT for 3 days. The reaction was stopped and concentrated to dryness. Crude residue was adsorbed on silica gel and purified by Combiflash column chromatography (40 g column, 0-3% MeOH/DCM) to provide 1-(2-hydroxy-4-((tetrahydro-2H-pyran-2-yl)oxy)phenyl)-2-(2-iso-propylphenyl)ethanone (316 mg, 51%) as an off-white solid. ¹H NMR (300 MHz, DMSO-d₆) δ ppm 12.3 (s, 1H), 8.03 (d, J=8.7 Hz, 1H), 7.31-7.09 (m, 4H), 6.61 (dd, J=8.7, 2.4 Hz, 1H), 6.55 (d, J=2.4 Hz, 1H), 5.61 (bs, 1H), 4.45 (s, 2H), 3.71-3.55 (m, 2H), 2.80 (sept, J=7.2 Hz, 1H), 1.83-1.53 (m, 6H), 1.10 (d, J=6.3 Hz, 6H). m/z=355 (M+H)⁺.

### Step 3: Preparation of 3-(2-iso-propylphenyl)-2-(4-((1-propylazetidin-3-yl)oxy)phenyl)-7-((tetrahydro-2H-pyran-2-yl)oxy)chroman-4-one

A mixture of 4-((1-propylazetidin-3-yl)oxy)benzaldehyde (265 mg, 1.2 mmol, 1.2 equiv.), 1-(2-hydroxy-4-((tetrahydro-2H-pyran-2-yl)oxy)phenyl)-2-(2-iso-propylphenyl)ethanone (351 mg, 1.0 mmol, 1.0 equiv.), piperidine (0.119 mL, 1.2 mmol, 1.2 equiv.) and DBU (0.098 mL, 0.7 mmol, 0.7 equiv.) in 2-butanol (8.000 mL) in a round bottom flask equipped with a Dean-Stark apparatus and condenser was heated at 130° C. Half the volume of 2-butanol (4 mL) was collected over 2 h, and the reaction was further heated at 130° C. for 12 h. Progress of the reaction was monitored by HPLC. The reaction mixture was allowed to cool to RT and concentrated to dryness. Crude residue was adsorbed on silica gel and purified by Combiflash column chromatography (25 g column, 0-5% MeOH/DCM) affording 3-(2-iso-propylphenyl)-2-(4-((1-propylazetidin-3-yl)oxy)phenyl)-7-((tetrahydro-2H-pyran-2-yl)oxy)chroman-4-one (290 mg, 53%). m/z=556 (M+H)⁺.

### Step 4. Preparation of 3-(2-isopropylphenyl)-4-methyl-2-(4((1-propylazetidin-3-yl)oxy)phenyl)-7-((tetrahydro-2H-pyran-2-yl)oxy)chroman-4-ol

To a solution of 3-(2-iso-propylphenyl)-2-(4-((1-propylazetidin-3-yl)oxy)phenyl)-7-((tetrahydro-2H-pyran-2-yl)oxy)chroman-4-one (290 mg, 0.5 mmol, 1.0 equiv.) in THF (5.000 mL, 50.6 mmol, 97.0 equiv.) at 0° C. was added methylmagnesium chloride 3.0 M solution in THF (0.870 mL, 4.2 mmol, 5.0 equiv.) dropwise. The ice bath was removed, and the reaction mixture was stirred for 12 h and allowed to warm to RT. The mixture was then cooled in an ice bath and additional methylmagnesium chloride (3.0 M, 0.870 mL, 5.0 equiv)) was added. The ice bath was removed, and the reaction mixture was allowed to warm to RT overnight. The reaction mixture was cooled to 0° C. and slowly quenched with MeOH (15 mL) and stirred for 15 min. The mixture was concentrated to dryness. Crude residue was adsorbed on silica gel and purified by combiflash column chromatography (25 g column, 0-5% MeOH/DCM) afforded 66 mg of bottom spot off-white solid, which was used without further purification. m/z=572 (M+H)⁺.

### Step 5. Preparation of 3-(2-iso-propylphenyl)-4-methyl-2-(4-((1-propylazetidin-3-yl)oxy)phenyl)-2H-chromen-7-ol (Compound 119)

A round-bottom flask containing 3-(2-iso-propylphenyl)-4-methyl-2-(4-((1-propylazetidin-3-yl)oxy)phenyl)-7-((tetrahydro-2H-pyran-2-yl)oxy)chroman-4-ol (66 mg, 0.1 mmol, 1.0 equiv.) was charged with an 80% aqueous solution of acetic acid (0.200 mL, 3.5 mmol, 30.3 equiv.) and water (0.800 mL, 44.4 mmol, 384.7 equiv.), and the resulting yellowish solution was purged with nitrogen and heated at 90° C. for 45 min under nitrogen. TLC (DCM:MeOH=9:1) and HPLC analysis indicated the disappearance of starting material. The reaction was cooled to ambient temperature. The reaction mixture was concentrated to dryness under high vacuum with the water bath set at 35° C. The crude residue was adsorbed on silica gel and purified by column chromatography (25 g column, 0-10% MeOH/DCM) to afford the desired product (30 mg, 59%).

The S and R isomers are separated in a similar manner as above.

### Example 24: Preparation of 3-(2-chlorophenyl)-4-methyl-2-(4-((1-propylazetidin-3-yl)oxy)phenyl-2H-chromen-7-ol (Compound 120)

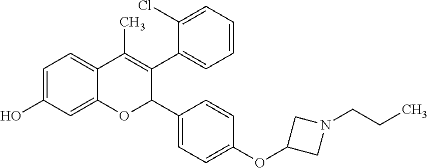 [SMILES: CCCN1CC(Oc2ccc(C3Oc4cc(O)ccc4C(C)=C3c3ccccc3Cl)cc2)C1]

### Step 1: Preparation of 3-(2-chlorophenyl)-7-hydroxy-4-methyl-2H-chromen-2-one

A oven dried sealable flask was charged with anhydrous potassium carbonate (9.509 g, 68.8 mmol, 3.0 equiv.), 2-chlorophenylboronic acid (5.380 g, 34.4 mmol, 1.5 equiv.) and tetrakis-triphenylphosphine palladium (0) (1.325 g, 1.1 mmol, 0.1 equiv., 10 mol %) and 100 mL of toluene:EtOH (2:1) and stirred for 5 min at RT. 3-Bromo-7-hydroxy-4-methyl-2H-chromen-2-one (5.850 g, 22.9 mmol, 1.0 equiv.), commercially available, was added and the mixture was heated at 90° C. under nitrogen overnight. The reaction was cooled to ambient temperature and diluted with EA and water. The organic layer was washed with brine, dried over anhydrous Na₂SO₄, filtered and concentrated. The resulting residue was dissolved in DCM and loaded to a silica gel column (120 g, 30% EA/Hex, then 1% MeOH/DCM) to give a light brown material which was triturated with MeOH to afford the title compound (3.30 g, 50.2%) as an off-white solid.

¹H NMR (300 MHz, CDCl₃), δ 10.62 (s, 1H), 7.71 (d, J=9.0 Hz, 1H), 7.60-7.57 (m, 1H), 7.46-7.35 (m, 3H), 6.86 (dd, J=9.0, 2.4 Hz, 1H), 6.78 (d, J=1.8 Hz, 1H), 2.12 (s, 3H).

### Step 2. Preparation of 3-(2-chlorophenyl)-4-methyl-7-((tri-iso-propylsilyl)oxy)-2H-chromen-2-one

To a solution of 3-(2-chlorophenyl)-7-hydroxy-4-methyl-2H-chromen-2-one (2.645 g, 9.2 mmol, 1.0 equiv.) in DMF (10 mL), DIPEA (2.400 mL, 13.8 mmol, 1.5 equiv.) was added chlorotri-iso-propylsilane (2.400 mL, 11.2 mmol, 1.2 equiv.) slowly at 22° C. The reaction mixture was stirred at ambient temperature for 2 h. TLC (10% EA/Hex) indicated the reaction was complete. The mixture was diluted with EA (200 mL) and washed with water (3×100 mL), then brine (100 mL), dried over anhydrous Na₂SO₄, filtered and concentrated. The resulting oil was dissolved in DCM and loaded to a silica gel column (25 g, 0-20% EA/Hex) to afford the title compound as a white solid (4 g, 97%).

¹H NMR (300 MHz, CDCl₃) δ 7.56-7.49 (m, 2H), 7.35 (dd, J=6.0, 3.3 Hz, 2H), 7.28-7.26 (m, 2H), 6.89-6.85 (m, 2H), 2.20 (s, 3H), 1.34-1.27 (m, 3H), 1.13 (d, J=7.2 Hz, 18H).

### Step 3. Preparation of 3-(2-chlorophenyl)-4-methyl-7-((tri-iso-propylsilyl)oxy)-2H-chromen-2-ol

A stirred solution of 3-(2-chlorophenyl)-4-methyl-7-((triisopropylsilyl)oxy)-2H-chromen-2-one (0.480 g, 1.1 mmol, 1.0 equiv.) in toluene (9.000 mL, 85.0 mmol, 78.4 equiv.) in a 250 mL 3-neck round bottom flask was cooled to −78° C. in a dry ice/acetone bath. To the reaction mixture was slowly added diisobutylaluminum hydride in cyclohexane (1.200 mL, 1.2 mmol, 1.1 equiv.) over 16 min maintaining an internal temperature below −74.3° C. The reaction was stirred for 1 h. An aliquot of sample was taken and quenched with MeOH and Rochelle's salt solution and extracted with EA. TLC (100/EA/Hex) indicated the reaction was complete. The reaction was quenched with MeOH (0.5 mL) and diluted with DCM (30 mL) and stirred at −78° C. for 10 min. Rochelle's salt solution was then added. The mixture was allowed to thaw and the phases were separated. The organic layer was washed with Rochelle's salt solution again, then brine, dried over anhydrous Na₂SO₄ for 20 min. The colorless solution was passed through a DCM equilibrated silica gel plug. The filtrate was concentrated in vacuo at 6° C. The oil was dissolved in DCM and transferred to a small round bottom flask (100 mL) and concentrated in vacuo. The resulting oil was dissolved in acetonitrile. The solution was frozen at −78° C. and was placed on a lyopholizer overnight to afford the desired product as a viscous oil (0.32 g, 66%).

¹H NMR (300 MHz, DMSO-d) δ 7.57-7.53 (m, 1H), 7.44-7.35 (m, 2H), 7.33 (d, J=8.1 Hz, 1H), 7.06 (d, J=6.3 Hz, 1H), 6.56 (dd, J=8.4, 2.4 Hz, 1H), 6.45 (d, J=2.4 Hz, 1H), 5.70 (d, J=6.3 Hz, 1H), 1.81 (s, 3H), 1.30-1.20 (m, 3H), 1.08 (d, J=6.9 Hz, 18H).

### Step. 4. Preparation of 3-(4-iodophenoxy)-1-propylazetidine

A solution of 4-iodophenol (3.200 g, 14.5 mmol, 1.0 equiv.), triphenylphosphine (5.722 g, 21.8 mmol, 1.5 equiv.), DIPEA (3.800 mL, 21.8 mmol, 1.5 equiv.), and 96.0% 1-propylazetidin-3-ol (2.617 g, 21.8 mmol, 1.5 equiv.) in THF (60 mL) was cooled to 0° C. Diisopropyl azodicarboxylate (4.522 mL, 21.8 mmol, 1.5 equiv.) was added dropwise and the mixture was allowed to warm to RT and was stirred overnight. TLC (20%, 50% EA/Hex) indicated 4-iodophenol was almost consumed (faint on TLC under UV). The mixture was concentrated and loaded on to a silica gel column (100 g, 0-100% EA/Hex). The product fractions were concentrated and treated with 4M HCl in dioxane at 0° C. to afford the title compound as the HCl salt. The white solid was then added saturated aq. sodium bicarbonate and stirred at RT for 30 min. It was then extracted with EA. The organic layer was washed with brine, dried over anhydrous sodium sulfate, filtered and concentrated to afford the desired product.

¹H NMR (300 MHz, CDCl₃), 7.53 (d, J=8.7 Hz, 2H), 6.55 (d, J=9.0 Hz, 2H), 4.73 (pent, J=5.7 Hz, 1H), 3.81-3.77 (m, 2H), 3.05 (t, J=7.1 Hz, 2H), 2.46 (t, J=7.5 Hz, 2H), 1.45-1.33 (m, 2H), 0.90 (t, J=7.5 Hz, 3H).

### Step. 5. Preparation of 3-(2-chlorophenyl)-4-methyl-2-(4-((1-propylazetidin-3-yl)oxy)phenyl)-2H-chromen-7-ol (Compound 120)

A solution of 3-(4-iodophenoxy)-1-propylazetidine (0.479 g, 1.5 mmol, 2.1 equiv.) in TH F (1.5 mL) was cooled in a dry ice-acetone bath. To the cooled solution in hexanes was added dropwise n-butyllithium (0.900 mL, 1.4 mmol, 1.9 equiv.) under nitrogen atmosphere at −78° C. After the addition was complete, the mixture was allowed to stir at the same temperature for 30 min. An aliquot of sample was taken and quenched with MeOH and concentrated in vacuo. ¹H NMR indicated that the metal-halogen exchange reaction was complete. 3-(2-chlorophenyl)-4-methyl-7-((tri-iso-propylsilyl)oxy)-2H-chromen-2-ol (0.320 g, 0.7 mmol, 1.0 equiv.) was dissolved in THF (2 mL) and the resulting solution was added dropwise to the above organolithium mixture at −78° C. over 5 min. The reaction mixture became bright yellow suspension. The dry ice acetone bath was removed and the reaction mixture was allowed to warm to room temperature. As the reaction warmed to −40° C., the solids dissolved resulting in a yellow solution. The reaction mixture was stirred at RT for 1 h.

The reaction was quenched with MeOH and the resulting mixture concentrated. The resulting red residue was dissolved in DCM and silica gel (˜1 g) was added and co-evaporated in vacuo. The material was purified on a silica gel column (12 g, 0-10% MeOH/DCM). The resulting red oil (280 mg) was taken up in THF (20 mL), and cooled to 0° C. Concentrated HCl (0.500 mL, 6.0 mmol, 8.3 equiv.) was added to the above solution and the resulting mixture stirred at room temp. for 1.5 h. The reaction was concentrated and sat'd NaHCO₃ solution (25 mL) and EA (25 mL) were added. The mixture was vigorously stirred at RT for 15 min. The mixture was then transferred to a separatory funnel and the phases were separated. The aqueous layer was extracted with EA (2×25 mL) and combined organic phases were washed with brine (50 mL), dried over anhydrous Na₂SO₄, filtered and concentrated. The red-purple residue was dissolved in DCM with small amount of MeOH and co-evaporated with silica gel (˜1 g) and purified on a silica gel column (12 g, 0-10% MeOH/DCM) to afford a dark brown oil which showed the desired mass (m/z=618 [M+H]⁺, 220 mg) for the cyclized material, and was used directly without further purification in the deprotection step.

The material was dissolved in THF (1 mL) and cooled to 0° C. and tetra-n-butylammonium fluoride (1.000 ml, 1.0 mmol, 1.4 equiv., 1 M in THF) was added. The reaction was stirred at 0° C. for 20 min, LCMS indicated the reaction was complete. The reaction was quenched with methanol and concentrated. The resulting red residue was dissolved in DCM and loaded to a silica gel column (12 g, 0-10%, held at 7.5% MeOH/DCM) to afford the title compound as a pink solid. The solid was dissolved in DCM and loaded to a preparative TLC plate and eluted with 5% MeOH/DCM. The desired band was collected, and stirred in MeOH/DCM for 10 min, and the silica gel was filtered off and the solids rinsed with 5% MeOH/DCM. The filtrate was concentrated and co-evaporated with MeOH three times before being dried in a vacuum oven at 60° C. overnight to afford Compound 120 (90 mg, 27% over three steps). HPLC (0-90% CH₃CN/H₂O/0.1% TFA) give one sharp peak at 10.31 min over a 20 min run. ¹H NMR (300 MHz, CDCl₃), δ 7.43-7.37 (m, 1H), 7.20-7.15 (m, 4H), 7.06 (t, J=7.4 Hz, 1H), 6.77 (dd, J=7.5, 1.2 Hz, 1H), 6.61-6.53 (m, 2H), 6.40 (dd, J=8.4, 2.1 Hz, 1H), 6.29-6.27 (m, 1H), 5.83 (s, 1H), 4.75-4.68 (m, 1H), 3.83 (apparent t, 2H), 3.11 (apparent q, 2H), 2.52 (t, J=7.5 Hz, 2H), 1.88 (s with a shoulder, 3H), 1.45-1.38 (m, 2H), 0.89 (t, J=7.4 Hz, 3H). m/z=462.1 [M+H]⁺

The S and R isomers are separated in a similar manner as above.

### Pharmaceutical Compositions

A compound of Formula I, Formula IA, Formula II, Formula IIA or Formula III can be provided if desired as a pharmaceutically acceptable salt, solvate, hydrate, prodrug, stereoisomer, tautomer, or a pharmaceutically acceptable composition thereof to treat a disorder that is mediated, modulated or affected by an estrogen receptor, including those treatable with an anti-estrogenic compound with virtually no estrogenic effect.

In one embodiment, a compound of Formula I, Formula IA, Formula II, Formula IIA or Formula III or its pharmaceutically acceptable salt can be administered in a pharmaceutical composition suitable for oral delivery to the patient, typically a human. Alternatively, a compound of Formula I, Formula IA, Formula II, Formula IIA or Formula III or its pharmaceutically acceptable salt can be delivered in a carrier suitable for topical, transdermal (including by patch), intravenous, parenteral, intraaortal, subcutaneous or other desired delivery route, including any method of controlled delivery, for example, using degradable polymers, or with nano or microparticles, liposomes, layered tablets or other structural frameworks which slow delivery.

In yet another aspect, the compound of Formula I, Formula IA, Formula II, Formula IIA or Formula III or its pharmaceutically acceptable salt can be used to prevent a disorder modulated through the estrogen receptor, which comprises administering to a patient in need of such prevention, a prophylactically effective amount of a compound or pharmaceutical composition.

The compound of Formula I, Formula IA, Formula II, Formula IIA or Formula III can be in the form of a salt. It can be administered as a pharmaceutically acceptable salt, for example, a pharmaceutically acceptable acid addition salt, including a hydrochloride, hydroiodide, hydrobromide, nitrate, sulfate, bisulfate, phosphate, acetate, lactate, citrate, tartrate, succinate, maleate, fumarate, benzoate, para-toluenesulfonate and the like.

The compound of Formula I, Formula IA, Formula II, Formula IIA or Formula III or its pharmaceutically acceptable salt is used to treat or prevent a disorder modulated by the estrogen receptor in an animal, typically a mammal, and most typically a human.

In yet another aspect, the present disclosure provides a combination of a compound of Formula I, Formula IA, Formula II, Formula IIA or Formula III or its pharmaceutically acceptable salt, and another pharmacologically active agent.

The selected compound of Formula I, Formula IA, Formula II, Formula IIA or Formula III or its pharmaceutically acceptable salt can also be used as adjunctive therapy or combination therapy with another active agent (drug or other compound that has therapeutic or prophylactic properties). For example, a therapeutically effective amount of the compound of Formula I, Formula IA, Formula II, Formula IIA or Formula III or its pharmaceutically acceptable salt can be used in combination with another anti-cancer agent, especially for estrogen receptor positive breast cancer, and in some embodiments, for estrogen receptor negative breast cancer.

In one aspect, the present disclosure provides a pharmaceutical composition comprising a pharmaceutically effective amount of a compound of Formula I, Formula IA, Formula II, Formula IIA or Formula III or its pharmaceutically acceptable salt and a pharmaceutically acceptable carrier.

The compound of Formula I, Formula IA, Formula II, Formula IIA and Formula III or its pharmaceutically acceptable salt provided herein are administered for medical therapy in a therapeutically effective amount. The amount of the compound of Formula I, Formula IA, Formula II, Formula IIA or Formula III or its pharmaceutically acceptable salt administered will typically be determined by a physician, in the light of the relevant circumstances, including the condition to be treated, the chosen route of administration, the compound or salt administered, the age, weight, and response of the individual patient, the severity of the patient's symptoms, and the like.

The pharmaceutical compositions provided herein can be administered by a variety of routes including oral, topical, systemic, parenteral, rectal, transdermal, subcutaneous, intravenous, intramuscular, and intranasal with a pharmaceutical carrier suitable for such administration. In one embodiment, the compound of one of the Formulas or its pharmaceutically acceptable salt is administered in a controlled release formulation.

Described herein below are various formulations comprised of a compound of Formula I, Formula IA, Formula II, Formula IIA or Formula III or its pharmaceutically acceptable salts. The formulation includes the active ingredient, as either a weight ratio or as a weight amount. It is to be understood, unless indicated to the contrary, that the weight amount and weight ratios are based upon the molecular weight of the compound of Formula I, Formula IA, Formula II, Formula IIA or Formula III, even if the formulation contains the salt form thereof.

The compositions for oral administration can take the form of bulk liquid solutions or suspensions, or bulk powders. Typically, the compositions are presented in unit dosage forms to facilitate accurate dosing. The term “unit dosage forms” refers to physically discrete units suitable as unitary dosages for human subjects and other mammals, each unit containing a predetermined quantity of active material of the compound of Formula I, Formula IA, Formula II, Formula IIA or Formula III or its pharmaceutically acceptable salt calculated to produce the desired therapeutic effect, in association with a suitable pharmaceutical excipient.

Typical unit dosage forms include prefilled, premeasured ampules or syringes of the liquid compositions or pills, tablets, capsules or the like in the case of solid compositions. In such compositions, the compound of Formula I, Formula IA, Formula II, Formula IIA or Formula III or its pharmaceutically acceptable salt may be present as a minor component (as a nonlimiting example, from about 0.1 to about 50% by weight or preferably from about 1 to about 40% by weight) with the remainder being various vehicles or carriers and processing aids helpful for forming the desired dosing form.

Liquid forms suitable for oral administration may include a suitable aqueous or nonaqueous vehicle with buffers, suspending and dispensing agents, colorants, flavors and the like. Solid forms may include, for example, any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate; a glidant such as colloidal silicon dioxide; a sweetening agent such as sucrose or saccharin; or a flavoring agent such as peppermint, methyl salicylate, or orange flavoring.

Injectable compositions comprised of a compound of Formula I, Formula IA, Formula II, Formula IIA or Formula III or its pharmaceutically acceptable salts are contemplated within the present disclosure. These injectable solutions use injectable carriers known within the art, such as injectable sterile saline or phosphate-buffered saline carriers and the like.

Transdermal compositions are typically formulated as a topical ointment or cream containing the compound of Formula I, Formula IA, Formula II, Formula IIA or Formula III or its pharmaceutically acceptable salt, for example in an amount ranging from about 0.01 to about 20% by weight, in another embodiment, from about 0.1 to about 20% by weight, in still another embodiment, from about 0.1 to about 10% by weight, and in still a different embodiment from about 0.5 to about 15% by weight. When formulated as an ointment, the compound of Formula I, Formula IA, Formula II, Formula IIA or Formula III or its pharmaceutically acceptable salt will typically be combined with either a suitable delivery polymeric composition, or a paraffinic or a water-miscible ointment base. Alternatively, the compound of Formula I, Formula IA, Formula II, Formula IIA or Formula III or its pharmaceutically acceptable salt may be formulated in a cream with, for example an oil-in-water cream base. Such transdermal formulations are well-known in the art and generally include additional ingredients to enhance the dermal penetration of stability of the active ingredients or the formulation. All such known transdermal formulations and ingredients are included within the scope provided herein.

The compound of Formula I, Formula IA, Formula II, Formula IIA and Formula III or its pharmaceutically acceptable salt can be administered by a transdermal device. Transdermal administration can be accomplished using a patch either of the reservoir or porous membrane type, or of a solid matrix variety.

The above-described components for orally administrable, injectable or topically administrable compositions are merely representative. Other materials as well as processing techniques and the like are set forth in Part 8 of Remington's Pharmaceutical Sciences, 17th edition, 1985, Mack Publishing Company, Easton, Pa., which is incorporated herein by reference.

The compound of Formula I, Formula IA, Formula II, Formula IIA or Formula III or its pharmaceutically acceptable salt can also be administered in sustained release forms or from sustained release drug delivery systems. A description of representative sustained release materials can be found in Remington's Pharmaceutical Sciences.

In certain embodiments, the formulation comprises water. In another embodiment, the formulation comprises a cyclodextrin derivative. In certain embodiments, the formulation comprises hexapropyl-β-cyclodextrin. In a more particular embodiment, the formulation comprises hexapropyl-β-cyclodextrin (10-50% in water). In a more particular embodiment, the formulation comprises Captisol®.

The present disclosure also includes pharmaceutically acceptable acid addition salts of compounds of Formula I, Formula IA, Formula II, Formula IIA or Formula III. The acids which are used to prepare the pharmaceutically acceptable salts are those which form non-toxic acid addition salts, i.e. salts containing pharmacologically acceptable anions such as the hydrochloride, hydroiodide, hydrobromide, nitrate, sulfate, bisulfate, phosphate, acetate, lactate, citrate, tartrate, succinate, maleate, fumarate, benzoate, para-toluenesulfonate, and the like.

The following formulation examples illustrate non-limiting representative pharmaceutical compositions that may be prepared in accordance with this disclosure for the purpose of illustration only. The present invention is specifically not limited to the following pharmaceutical compositions. Although the examples in the formulations hereinbelow refer to compounds of Formula I, Formula IA, Formula II, Formula IIA and Formula III, it is understood that the pharmaceutically acceptable salts thereof may be used in their stead. Thus, for example, if the compound of Formula I, Formula IA, Formula II, Formula IIA or Formula III is present in the formulation as its salt, the weight ratio is to be based upon the weight of the compound of Formula I, Formula IA, Formula II, Formula IIA or Formula III present in the formulation without taking into account the weight attributable to the salt thereof.

### Formulation 1—Tablets

A compound of Formula I, Formula IA, Formula II, Formula IIA or Formula III may be admixed as a dry powder with a dry gelatin binder in an approximate 1:2 weight ratio. A minor amount of magnesium stearate is added as a lubricant. The mixture is formed into 240-270 mg tablets (80-90 mg of a compound of Formula I, Formula IA, Formula II, Formula IIA or Formula III per tablet) in a tablet press.

### Formulation 2—Capsules

A compound of Formula I, Formula IA, Formula II, Formula IIA or Formula III may be admixed as a dry powder with a starch diluent in an approximate 1:1 weight ratio. The mixture is filled into 250 mg capsules (125 mg of a compound of Formula I, Formula IA, Formula II, Formula IIA or Formula III per capsule).

### Formulation 3—Liquid

A compound of Formula I, Formula IA, Formula II, Formula IIA or Formula III (125 mg) may be admixed with sucrose (1.75 g) and xanthan gum (4 mg) and the resultant mixture may be blended, passed through a No. 10 mesh U.S. sieve, and then mixed with a previously made solution of microcrystalline cellulose and sodium carboxymethyl cellulose (11:89, 50 mg) in water. Sodium benzoate (10 mg), flavor, and color are diluted with water and added with stirring. Sufficient water may then be added to produce a total volume of 5 mL.

### Formulation 4—Tablets

A compound of Formula I, Formula IA, Formula II, Formula IIA or Formula III can be admixed as a dry powder with a dry gelatin binder in an approximate 1:2 weight ratio. A minor amount of magnesium stearate is added as a lubricant. The mixture is formed into 450-900 mg tablets (150-300 mg of a compound of Formula I, Formula IA, Formula II, Formula IIA or Formula III) in a tablet press. In other embodiments, there is between 10 and 500 mg of a compound of Formula I, Formula IA, Formula II, Formula IIA or Formula III in the oral tablet.

### Formulation 5—Injection

A compound of Formula I, Formula IA, Formula II, Formula IIA or Formula III can be dissolved or suspended in a buffered sterile saline injectable aqueous medium to a concentration of approximately 5, or 10, or 15, or 20, or 30 or 50 mg/mL.

### Formulation 6—Tablets

A compound of Formula I, Formula IA, Formula II, Formula IIA or Formula III may be admixed as a dry powder with a dry gelatin binder in an approximate 1:2 weight ratio. A minor amount of magnesium stearate is added as a lubricant. The mixture is formed into 90-150 mg tablets (30-50 mg of a compound of Formula I, Formula IA, Formula II, Formula IIA or Formula III per tablet) in a tablet press.

### Formulation 7—Tablets

A compound of Formula I, Formula IA, Formula II, Formula IIA or Formula III may be admixed as a dry powder with a dry gelatin binder in an approximate 1:2 weight ratio. A minor amount of magnesium stearate is added as a lubricant. The mixture is formed into 30-90 mg tablets (10-30 mg of a compound of Formula I, Formula IA, Formula II, Formula IIA or Formula III per tablet) in a tablet press.

### Formulation 8—Tablets

A compound of Formula I, Formula IA, Formula II, Formula IIA or Formula III may be admixed as a dry powder with a dry gelatin binder in an approximate 1:2 weight ratio. A minor amount of magnesium stearate is added as a lubricant. The mixture is formed into 0.3-30 mg tablets (0.1-10 mg of a compound of Formula I, Formula IA, Formula II, Formula IIA or Formula III per tablet) in a tablet press.

### Formulation 9—Tablets

A compound of Formula I, Formula IA, Formula II, Formula IIA or Formula III may be admixed as a dry powder with a dry gelatin binder in an approximate 1:2 weight ratio. A minor amount of magnesium stearate is added as a lubricant. The mixture is formed into 150-240 mg tablets (50-80 mg of a compound of Formula I, Formula IA, Formula II, Formula IIA or Formula III per tablet) in a tablet press.

### Formulation 10—Tablets

A compound of Formula I, Formula IA, Formula II, Formula IIA or Formula III may be admixed as a dry powder with a dry gelatin binder in an approximate 1:2 weight ratio. A minor amount of magnesium stearate is added as a lubricant. The mixture is formed into tablets (5-1000 mg of a compound of Formula I, Formula IA, Formula II, Formula IIA or Formula III per tablet) in a tablet press.

### Use of Compounds in Medical Therapy

A compound of Formula I, Formula IA, Formula II, Formula IIA or Formula III described herein or its salt or pharmaceutically acceptable composition as described herein are complete anti-estrogens useful to treat any disorder modulated, mediated or affected by the estrogen receptor.

In one embodiment, the compound of Formula I, Formula IA, Formula II, Formula IIA or Formula III or its pharmaceutically acceptable salt is used in combination or alternation with another anti-cancer agent for the treatment of cancer, as described more fully below. In another embodiment, the compound of Formula I, Formula IA, Formula II, Formula IIA or Formula III or its pharmaceutically acceptable salt is used in combination or alternation with estrogen or a partial estrogen receptor antagonist for the treatment of a postmenopausal disorder, also described below.

In one embodiment, a compound of Formula I, Formula IA, Formula II, Formula IIA or Formula III or its pharmaceutically acceptable salt is used to treat local, advanced or metastatic breast cancer that is positive for expression of estrogen receptors, progesterone receptors or both (receptor positive advanced breast cancer). In an alternative embodiment, the compound of Formula I, Formula IA, Formula II, Formula IIA or Formula III or its pharmaceutically acceptable salt is used to treat estrogen or progesterone receptor negative breast cancer. The compound of Formula I, Formula IA, Formula II, Formula IIA or Formula III or its pharmaceutically acceptable salt can be used as the initial treatment of advanced breast cancer in patients who have never received previous hormonal therapy for advanced breast cancer, either by itself or in combination with one or more other anti-cancer agents described below or otherwise known to those skilled in the art. It is also useful for second line therapy for treatment after a previous hormonal therapy has failed, either by itself or in combination with another anticancer agent, for example, a targeted therapy such as an mTOR inhibitor such as everolimus.

The compounds of Formula I, Formula IA, Formula II, Formula IIA or Formula III or their pharmaceutically acceptable salts are also useful as adjunctive therapy after or instead of chemotherapy, radiation or surgery. Such adjuvant use is often used for several years, perhaps 5 years, after chemotherapy or other therapies have been concluded, but may optimally be continued for additional years.

The compounds of Formula I, Formula IA, Formula II, Formula IIA or Formula III or their pharmaceutically acceptable salts are also useful for the prevention of breast cancer in women at high risk and can be taken for any desired time period, including indefinitely. For example, a patient, typically a woman, with a family history of breast cancer, or who has been determined to carry a mutation in the BRACA1 or BRACA2 gene or other genes that predispose a patient to breast cancer may choose to use such preventative treatment instead of a mastectomy or other intervention. The compounds of Formula I, Formula IA, Formula II, Formula IIA and Formula III or their pharmaceutically acceptable salts described herein are also useful as neoadjuvants to shrink large tumors prior to surgical removal, both to enable breast conservative surgery and to reduce the risk of recurrence. In addition to breast cancer these compounds of Formula I, Formula IA, Formula II, Formula IIA and Formula III or their pharmaceutically acceptable salts also are useful in treating other cancers and other overgrowth diseases of the female reproductive tract including ovarian, endometrial, and vaginal cancer and endometriosis. Besides these reproductive tissues the compounds of Formula I, Formula IA, Formula II, Formula IIA and Formula III or their pharmaceutically acceptable salts are useful in treating lung cancers that are positive for estrogen or progesterone receptors.

The compounds of Formula I, Formula IA, Formula II, Formula IIA and Formula III or their pharmaceutically acceptable salts or compositions thereof can be used in conjunction with selective estrogen receptor modulators (SERMS) referred to herein and together they are useful for hormonal therapy for postmenopausal women in particular to treat or prevent osteoporosis. In one embodiment, a compound of Formula I, Formula IA, Formula II, Formula IIA or Formula III or its pharmaceutically acceptable salt is used in combination with an estrogen, SERM or partial anti-estrogen whereby the complete anti-estrogen prevents adverse action of the total or partial estrogen on the uterus and other tissues.

The present compounds of Formula I, Formula IA, Formula II, Formula IIA or Formula III and their pharmaceutically acceptable salts are used as therapeutic or prophylactic agents for the treatment of conditions in mammals, particularly humans that are modulated by estrogen receptors.

An oral pharmaceutical composition comprised of a compound of Formula I, Formula IA, Formula II, Formula IIA or Formula III or its pharmaceutically acceptable salts thereof are useful for treating locally advanced or metastatic breast cancer, preventing recurrence or early breast cancer after surgery, and preventing breast cancer in women at high risk. They are also useful for treating all estrogen-dependent cancers of the reproductive tract including endometrial and ovarian cancers. They can be used in the treatment of lung and bronchial cancers that express estrogen receptors.

Selective estrogen receptor modulators (SERMS) such as tamoxifen, raloxifene, lasofoxifene, and bazedoxifene additionally have application as hormone replacement therapy to prevent osteoporosis and other disorders such as hot flashes, etc. in post-menopausal women, a use that depends on their partial estrogen like action, for example, on bone. The compound of Formula I, Formula IA, Formula II, Formula IIA and Formula III or its pharmaceutically acceptable salts described herein can be employed in combination with an estrogen or a selective estrogen receptor modulator to block the unwanted estrogenic activity of the therapy. The complete anti-estrogen is dosed in the amount to prevent the adverse action of the estrogen or estrogen receptor modulator on the uterus and mammary gland yet allowing the beneficial action of estrogen on bone and vasomotor symptoms.

The compounds of Formula I, Formula IA, Formula II, Formula IIA and Formula III or their pharmaceutically acceptable salts can be administered for the treatment of cancer, and in particular breast cancer in combination or association with Herceptin, Tykerb, CDK4/6 inhibitor such as PD-0332991, mTOR inhibitor such as Novartis' everolimus and other rapamycin analogs such as rapamycin and temsirolimus, Millennium's MLN0128 TORC1/2 inhibitor, an EFGR-family inhibitor such as trastuzumab, pertuzumab, ado-trastuzumab emtansine, erlotinib, gefitinib, neratinib and similar compounds, a PI3 Kinase Inhibitor such as perifosine, CAL101, BEZ235, XL147, XL765, GDC-0941, and IPI-145, a histone deacetylase inhibitor such as vorinostat, romidepsin, panobinostat, valproic acid, etinostat, and belinostat.

In another method of treatment aspect, provided herein is a method of treating a mammal susceptible to or afflicted with a condition influenced by estrogen receptor by administering to a subject in need thereof a compound of Formula I, Formula IA, Formula II, Formula IIA or Formula III or its pharmaceutically acceptable salt thereof.

In another embodiment, the compounds of Formula I, Formula IA, Formula II, Formula IIA and Formula III or their pharmaceutically acceptable salts are provided for use in medical therapy, including for any of the conditions described herein. The use of the compounds of Formula I, Formula IA, Formula II, Formula IIA and Formula III or their pharmaceutically acceptable salts in the manufacture of a medicament for the treatment or prevention of one of the aforementioned conditions and diseases is also contemplated to be within the purview of this disclosure.

Injection dose levels of injectable solutions comprised of compounds of Formula I, Formula IA, Formula II, Formula IIA or Formula III or their pharmaceutically acceptable salts are provided in any desired dosage, for example, from about 0.1 mg/kg/hour to at least 10 mg/kg/hour, all for from about 1 to about 120 hours and especially 24 to 96 hours. In one embodiment, a preloading bolus of from about 0.1 mg/kg to about 10 mg/kg or more comprised of the compounds of Formula I, Formula IA, Formula II, Formula IIA or Formula III or their pharmaceutically acceptable salts may also be administered to achieve adequate steady state levels. The maximum total dose is not expected to exceed about 2 g/day for a 40 to 80 kg human patient.

For oral dosing, any dose is appropriate that achieves the desired goals. In one example, suitable daily dosages are between about 0.1-4000 mg, more typically between 5 mg and 1 gram, more typically between 10 mg and 500 mg, and administered orally once-daily, twice-daily or three times-daily, continuous (every day) or intermittently (e.g., 3-5 days a week). For example, when used to treat any disorder described herein, the dose of the compounds of Formula I or their pharmaceutically acceptable salts usually range between about 0.1 mg, more usually 10, 50, 100, 200, 250, 1000 or up to about 2000 mg per day.

For the prevention and/or treatment of long-term conditions, such as neurodegenerative and autoimmune conditions, the regimen for treatment usually stretches over many months or years. Oral dosing may be preferred for patient convenience and tolerance. With oral dosing, one to five and especially two to four and typically three oral doses per day are representative regimens. Using these dosing patterns, nonlimiting dosages might range from about 0.01 to about 20 mg/kg of the compound of Formula I, Formula IA, Formula II, Formula IIA or Formula III or its pharmaceutically acceptable salt provided herein, with preferred doses each providing from about 0.1 to about 10 mg/kg and especially about 1 to about 5 mg/kg.

Transdermal doses are generally selected to provide similar or lower blood levels than are achieved using injection doses.

When used to prevent the onset of cancer, a neurodegenerative, autoimmune or inflammatory condition, the compounds of Formula 1, Formula IA, Formula II, Formula IIA and Formula III or their pharmaceutically acceptable salts will be administered to a patient at risk for developing the condition, typically on the advice and under the supervision of a physician, at the dosage levels described above. Patients at risk for developing a particular condition generally include those that have a family history of the condition, or those who have been identified by genetic testing or screening to be particularly susceptible to developing the condition.

A compound of Formula I, Formula IA, Formula II, Formula IIA or Formula III or its pharmaceutically acceptable salts provided herein can be administered as the sole active agent or it can be administered in combination with other agents. Administration in combination can proceed by any technique apparent to those of skill in the art including, for example, separate, sequential, concurrent and alternating administration.

### Demonstration of the Activity of the Compounds of the Present Invention Using Sensitive In Vitro Estrogenicity Assays

### Example 25

Representative compounds were tested for their inhibitory activity of estrogen according to the assay methods described in Hodges-Gallagher, L., Valentine, C. V., El Bader, S. and Kushner, P. J. (2007) “Histone Deacetylase Inhibitors Enhance the Efficacy of Hormonal Therapy Agents on Breast Cancer Cells and Blocks Anti-estrogen-Driven Uterine Cell Proliferation” Breast Cancer Res Treat, November; 105(3):297-309. Specifically, an estrogen-responsive reporter gene (ERE-tk109-Luc) was transiently transfected into MCF-7 cells and treated with anti-estrogens in triplicate in the presence of 100 pM 17β-estradiol (E2) for 18-22 hours. Luciferase activity was normalized to activity of E2 alone and IC₅₀'s were calculated using the least squares fit method.

The assay results for inhibition of E2-induced transcription in breast cells (nM) are listed in Table 1.

TABLE 1

| Compound | IC₅₀ |
|---|---|
| 101 | 3 |
| 102 | 2 |
| 103 | 27 |
| 104 | 2 |
| 105 | 4 |
| 106 | 54 |
| 107 | 5 |
| 108 | 1 |
| 109 | 109 |
| 110 | 14 |
| 111 | 165 |
| 112 | 2 |
| 113 | 13 |
| 114 | 5 |
| 115 | 10 |
| 116 | 5 |
| 117 | 4 |
| 118 | 254 |
| 119 | 15 |
| 120 | 8 |

### Example 26

Proliferation of MCF-7 breast cancer cells was measured using a fluorescent DNA binding dye 6-8 days after treatment in triplicate with representative compounds in the presence of 100 pM E2 in hormone-depleted medium. IC₅₀'s were calculated from individual experiments as in Example 25.

The assay results for inhibition of E2-stimulated proliferation in breast cells (nM) are listed in Table 2.

TABLE 2

| Compound | IC₅₀ |
|---|---|
| 101 | 7 |
| 102 | 6 |
| 103 | 59 |
| 104 | 6 |
| 105 | 10 |
| 106 | 114 |
| 107 | 13 |
| 108 | 9 |
| 109 | 40 |
| 110 | 216 |
| 111 | >300 |
| 112 | 7 |
| 113 | 24 |
| 114 | 14 |
| 115 | 15 |
| 116 | 5 |
| 117 | 33 |
| 118 | 132 |
| 119 | 33 |
| 120 | 10 |

### Example 27

ERα expression was detected in MCF-7 cell lysates treated with 100 nM antiestrogens in serum-free medium for 24 hours and immunoblotted with an antibody specific to ERα.

Band intensity was normalized relative to vehicle for each individual experiment and listed in Table 3 (Relative change ERα levels (% vehicle)).

TABLE 3

| Compound | % veh |
|---|---|
| 101 | 51 |
| 102 | 50 |
| 103 | 55 |
| 104 | 44 |
| 105 | 39 |
| 106 | 53 |
| 110 | 104 |
| 111 | 82 |
| 112 | 35 |
| 113 | 38 |
| 117 | 45 |
| 118 | 55 |
| 119 | 36 |
| 120 | 61 |

### Example 28

Method for performing the alkaline phosphatase (AP) assay. ECC-1 cells (American Type Culture Collection, Manassas, Va.) were maintained in RPMI medium plus 10% fetal bovine serum at 37° C. At the beginning of the assay trypsinized cells were resuspended in RPMI medium plus 5% charcoal dextran stripped serum (CDSS, (Hyclone, Logan, Utah)) and plated at a density of 25-50 k cells per well into a 96-well plate for at least 6 hours. Representative compounds were diluted in serum-free medium and added 1:1 to plated cells in replicate wells (2.5% CDSS final). Plates were incubated for 3 days at 37° C. and subsequently frozen at −80° C. to lyse cells after removing the medium. Thawed plates were incubated with a chromogenic substrate of AP, p-nitrophenyl phosphate (Invitrogen, Grand Island, N.Y.), for 40 minutes at 40° C. Absorbances were read at 405 nm using a plate spectrophotometer. AP activity was normalized to 500 pM 17β-estradiol (E2), reported as the maximum activity observed for each individual experiment, regardless of dose. This assay was shown to correlate with the in vivo studies comparing uterine wet weight in ovariectomized rats following treatment with a number of anti-estrogens. The assay results for induction of AP activity in uterine cells (% E2) are listed in Table 4.

TABLE 4

| | % |
|---|---|
| Compound | E2 |
| 101 | 9 |
| 102 | 9 |
| 103 | 23 |
| 104 | 5 |
| 105 | 4 |
| 106 | 17 |
| 107 | 3 |
| 108 | 3 |
| 109 | 18 |
| 110 | 35 |
| 111 | 32 |
| 112 | 5* |
| 113 | 17 |
| 114 | 15 |
| 115 | 20 |
| 116 | 9 |
| 117 | 16 |
| 118 | 104 |
| 119 | 44 |
| 120 | 12 |

### Example 29

AP activity was assayed as in Example 28 but cells were co-treated with 500 pM E2. The assay results are listed in Table 5.

TABLE 5

| Compound | % E2 |
|---|---|
| 101 | 8 |
| 102 | 8 |
| 103 | 36 |
| 104 | 4 |
| 105 | 4 |
| 106 | 44 |
| 107 | 4 |
| 108 | 4 |
| 109 | 42 |
| 110 | 36 |
| 111 | 61 |
| 112 | 4 |
| 113 | 15 |
| 114 | 23 |
| 115 | 42 |
| 116 | 24 |
| 117 | 8 |
| 118 | 16 |
| 119 | 18 |
| 120 | 14 |

The above preferred embodiments and examples were given to illustrate the scope and spirit of the present invention. These embodiments and examples will make apparent to those skilled in the art other embodiments and examples. The other embodiments and examples are within the contemplation of the present invention. Therefore, the present invention should be limited only by the amended claims.

What is claimed is:

1. A compound of Formula I 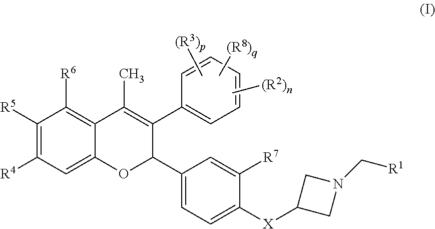
or a pharmaceutically acceptable salt thereof,
wherein
X is CH₂, O, S, NH, or N—(C₁-C₄ alkyl);
R¹ is —(C(Y¹)(Y²))ₘ—C((Y³)(Y⁴)(Y⁵)), wherein Y¹, Y², Y³, Y⁴ and Y⁵ are independently hydrogen or fluorine;
m is 0 or 1;
R² is hydrogen, halogen, cyano, or hydroxy;
R³ is hydrogen, halogen, C₁-C₄ alkyl, —CH₂F, —CHF₂, or —CF₃;
R⁴ and R⁵ are each independently hydrogen, halogen, or hydroxy, provided that R⁴ and R⁵ are not both hydroxy;
R⁶ and R⁷ are each independently hydrogen or halogen;
R⁸ is hydrogen, halogen, cyano, hydroxy, or C₁₋₄ alkyl;
n is 1 or 2;
p is 1 or 2; and
q is 1.

2. The compound or salt according to claim 1, wherein X is CH₂ or O.

3. The compound or salt according to claim 1, wherein m is 1.

4. The compound or salt according to claim 1, wherein R¹ is (CH₂)ₘ—C((Y³)(Y⁴)(Y⁵)).

5. The compound or salt according to claim 1, wherein R² is hydrogen, halogen, or hydroxy; R³ is hydrogen or C₁-C₄ alkyl; and R⁸ is hydrogen.

6. The compound or salt according to claim 1, wherein R⁴ is hydroxy, R⁵ is hydrogen and R⁶ is hydrogen.

7. The compound according to claim 1, having Formula IA 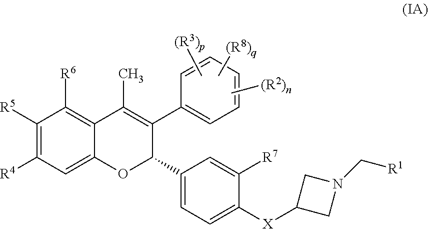
or a pharmaceutically acceptable salt thereof.

8. A compound of Formula II 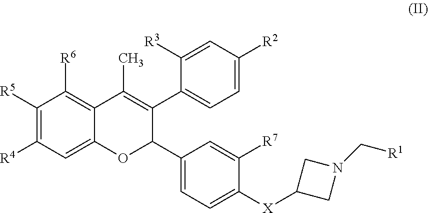
or a pharmaceutically acceptable salt thereof,
wherein
X is CH₂, O, S, NH, or N—(C₁-C₄ alkyl);
R¹ is —(C(Y¹)(2))ₘ—C((Y³)(Y⁴)(Y⁵)), wherein Y¹, Y², Y³, Y⁴, and Y⁵ are independently hydrogen or fluorine;
m is 0 or 1;
R² is hydrogen, halogen, cyano, or hydroxy;
R³ is hydrogen, halogen, C₁-C₄ alkyl, —CH₂F, —CHF₂, or —CF₃;
R⁴ and R⁵ are each independently hydrogen, halogen, or hydroxy, provided that R⁴ and
R⁵ are not both hydroxy; and
R⁶ and R⁷ are each independently hydrogen or halogen.

9. The compound or salt according to claim 8, wherein X is CH₂ or O.

10. The compound or salt according to claim 8, wherein X is O.

11. The compound or salt according to claim 8, wherein m is 1.

12. The compound or salt according to claim 8, wherein R¹ is (CH₂)ₘ—C((Y³)(Y⁴)(Y⁵)).

13. The compound or salt according to claim 8, wherein R¹ is —CH₂CH₃.

14. The compound or salt according to claim 8, wherein R² is hydroxy and R³ is hydrogen.

15. The compound or salt according to claim 8, wherein R⁴ is hydroxy, R⁵ is hydrogen and R⁶ is hydrogen.

16. The compound according to claim 8, having Formula IIA 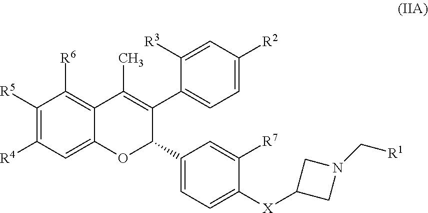
or a pharmaceutically acceptable salt thereof.

17. The compound according to claim 1, wherein the compound is selected from the group consisting of
3-(4-hydroxyphenyl)-4-methyl-2-(4-((1-propylazetidin-3-yl)methyl)phenyl)-2H-chromen-7-ol;
3-(4-hydroxyphenyl)-4-methyl-2-(4-((1-propylazetidin-3-yl)oxy)phenyl)-2H-chromen-7-ol;
2-(3-fluoro-4-((1-propylazetidin-3-yl)oxy)phenyl)-3-(4-hydroxyphenyl)-4-methyl-2H-chromen-7-ol;
3-(4-hydroxyphenyl)-4-methyl-2-(4-((1-(3,3,3-trifluoropropyl)azetidin-3-yl)oxy)phenyl)-2H-chromen-7-ol;
2-(3-fluoro-4-((1-propylazetidin-3-yl)methyl)phenyl)-3-(4-hydroxyphenyl)-4-methyl-2H-chromen-7-ol;
4-methyl-3-phenyl-2-(4-((1-propylazetidin-3-yl)oxy)phenyl)-2H-chromen-7-ol;
3-(4-fluorophenyl)-4-methyl-2-(4-((1-propylazetidin-3-yl)oxy)phenyl)-2H-chromen-7-ol;
3-(2-chloro-4-fluorophenyl)-4-methyl-2-(4-((1-propylazetidin-3-yl)oxy)phenyl)-2H-chromen-7-ol;
3-(2-isopropylphenyl)-4-methyl-2-(4-((1-propylazetidin-3-yl)oxy)phenyl)-2H-chromen-7-ol; and
3-(2-chlorophenyl)-4-methyl-2-(4-((1-propylazetidin-3-yl)oxy)phenyl)-2H-chromen-7-ol,
or a pharmaceutically acceptable salt thereof.

18. The compound or salt according to claim 17, in the S configuration.

19. A compound, which is 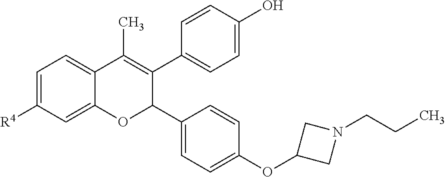
or a pharmaceutically acceptable salt thereof.

20. A compound, which is 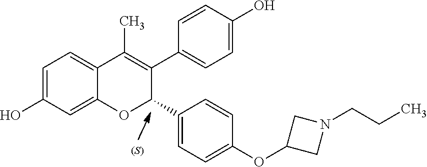
or a pharmaceutically acceptable salt thereof.

21. The pharmaceutical composition comprising a compound of claim 1, or a pharmaceutically acceptable salt thereof, and a pharmaceutically acceptable carrier therefor.

22. The pharmaceutical composition comprising a compound of claim 19 or a pharmaceutically acceptable salt thereof, and a pharmaceutically acceptable carrier therefor.

23. The pharmaceutical composition comprising a compound of claim 20 or a pharmaceutically acceptable salt thereof, and a pharmaceutically acceptable carrier therefor.

24. The method of treating a disorder mediated by the estrogen receptor in a patient, which comprises administering to the patient the compound according to claim 1, or a pharmaceutically acceptable salt thereof.

25. The method according to claim 24, wherein the disorder is breast cancer.

26. The method according to claim 24, wherein the disorder is selected from the group consisting of ovarian cancer, endometrial cancer, vaginal cancer, endometriosis and lung cancer.
